SEQUENCE LISTING (6 sequences)

SEQ ID NO: 1 - DNA, 1753 bp, Artificial Sequence
  Synthetic Polynucleotide
acatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttagatgaaagagaagatcttcaaaagaaaacttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttatctagccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattgcacaattttctaaggtaagaatttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttatctagccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattgcacaattttctaaggtaagaatttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttatctagccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttagatgaaagagaagatcttcaaaagaaaacttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 2 - DNA, 20 bp, Artificial Sequence
  Synthetic Polynucleotide
tacctaagcctcctggagca

SEQ ID NO: 3 - DNA, 23 bp, Artificial Sequence
  Synthetic Polynucleotide
cttttggcagtttttgccctgta

SEQ ID NO: 4 - DNA, 26 bp, Artificial Sequence
  Synthetic Polynucleotide
acgtagataagtagcatggcgggtta

SEQ ID NO: 5 - DNA, 23 bp, Artificial Sequence
  Synthetic Polynucleotide
agctcctatgttgttatctagag

SEQ ID NO: 6 - DNA, 23 bp, Artificial Sequence
  Synthetic Polynucleotide
ctaggggttccttgtagttaatg

(19) United States
(12) Patent Application Publication
(10) Pub. No.: US 2024/0261434 A1
(43) Pub. Date: Aug. 8, 2024

(54) PRODUCTS AND METHODS FOR TREATING MUSCULAR DYSTROPHY

(71) Applicant: RESEARCH INSTITUTE AT NATIONWIDE CHILDREN'S HOSPITAL, Columbus, OH (US)

(72) Inventors: Kevin Flanigan, Columbus, OH (US); Nicolas Sebastien Wein, Columbus, OH (US); Tabatha Simmons, Columbus, OH (US)

(21) Appl. No.: 18/246,594

(22) PCT Filed: Sep. 28, 2021
(86) PCT No.: PCT/US2021/052425

§ 371 Date: Mar. 24, 2023

Related U.S. Application Data
(60) Provisional application No. 63/084,259, filed on Sep. 28, 2020.

(51) Int. Cl. A61K 48/00; A61K 9/00; A61K 38/17; A61K 45/06; A61P 21/00; C12N 15/86

(52) U.S. Cl. CPC A61K 48/005; A61K 9/0019; A61K 38/1709; A61K 45/06; A61K 48/0075; A61K 48/0083; A61P 21/00; C12N 15/86; C12N 2750/14143

(57) ABSTRACT

Products and methods for treating or preventing muscular dystrophies in patients with duplications of exon (2) in their DMD gene or DMD mutations of any class that maintain a functional IRES sequence within exon (5), and an open reading frame from exon (6) though the end of the DMD gene are provided. Gene therapy vectors, such as adeno-associated virus (AAV) vectors and methods of using these vectors to express DMD are provided. The products and methods are used for treating and/or preventing muscular dystrophies, such as Duchenne Muscular Dystrophy or Becker Muscular Dystrophy.

### FIELD

The disclosure relates to the field of gene therapy for the treatment and/or prevention of muscular dystrophy. More particularly, the disclosure provides products and methods for treating or preventing muscular dystrophies in patients with duplications of exon 2 in their DMD gene or DMD mutations of any class that maintain a functional IRES sequence within exon 5, and an open reading frame from exon 6 though the end of the DMD gene. Gene therapy vectors, such as adeno-associated virus (AAV) vectors, and methods of using these vectors to express DMD are provided. The products and methods are used for treating and/or preventing muscular dystrophies such as Duchenne Muscular Dystrophy or Becker Muscular Dystrophy.

### INCORPORATION BY REFERENCE OF THE SEQUENCE LISTING

This application contains, as a separate part of disclosure, a Sequence Listing in computer-readable form (Filename: 55017_SeqListing.txt; 3,722 bytes—ASCII text file created Sep. 21, 2021) which is incorporated by reference herein in its entirety.

### BACKGROUND

Muscular dystrophies (MDs) are a group of genetic degenerative diseases primarily affecting voluntary muscles. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

The MDs are a group of diseases without identifiable treatment that gravely impact individuals, families, and communities. The costs are incalculable. Individuals suffer emotional strain and reduced quality of life associated with loss of self-esteem. Extreme physical challenges resulting from loss of limb function creates hardships in activities of daily living. Family dynamics suffer through financial loss and challenges to interpersonal relationships. Siblings of the affected feel estranged, and strife between spouses often leads to divorce, especially if responsibility for the muscular dystrophy can be laid at the feet of one of the parental partners. The burden of quest to find a cure often becomes a life-long, highly focused effort that detracts and challenges every aspect of life. Beyond the family, the community bears a financial burden through the need for added facilities to accommodate the handicaps of the muscular dystrophy population in special education, special transportation, and costs for recurrent hospitalizations to treat recurrent respiratory tract infections and cardiac complications. Financial responsibilities are shared by state and federal governmental agencies extending the responsibilities to the taxpaying community.

One form of MD is Duchenne Muscular Dystrophy (DMD). DMD, an X-linked degenerative muscle disorder, is the most common severe childhood form of muscular dystrophy affecting around 1:5200 male births (Mendell et al., Ann Neurol 71, 304-313 (2012)). Symptoms of generalized muscle weakness first appear at ages 3-5 and progress into a loss of ambulation by age 13, with death typically occurring in the third decade of life due to cardiomyopathy or respiratory insufficiency (Passamano et al., Acta Myol 31, 121-125 (2012); Duchenne, The Pathology of Paralysis with Muscular Degeneration (Paralysie Myosclerotique), or Paralysis with Apparent Hypertrophy. Br Med J 2, 541-542 (1867)). DMD is caused by mutations that disrupt the open reading frame in the DMD gene, which encodes dystrophin (Juan-Mateu et al., PLoS One 10, e0135189 (2015)), a large (427 kDa) multifunctional protein that is localized at the subsarcolemmal region of myofibers, where it plays an important role in protecting the sarcolemma from mechanical damage caused by muscle contraction (Petrof et al., Proc Natl Acad Sci USA 90, 3710-3714 (1993)).

Another form of MD is Becker Muscular Dystrophy (BMD). BMD is a milder allelic disorder which results from the presence of a partially functional dystrophin protein occurring from mutations that maintain an open reading frame (ORF) (Wein et al., Nature Medicine 20, 992-1000 (2014); Monaco, Trends Biochem Sci 14, 412-415 (1989)). BMD, like DMD, is a genetic disorder that gradually makes the body's muscles weaker and smaller. BMD affects the muscles of the hips, pelvis, thighs, and shoulders, as well as the heart, but is known to cause less severe problems than DMD. Because of the variety of in-frame mutations resulting in a variety of partially functional proteins, BMD has a broad phenotypic spectrum with, for example, loss of ambulation ranging from the late teenage years to late adulthood.

Promising therapeutic approaches to DMD are based on the replacement of a functional version of DMD, or its repair at the DNA or pre-mRNA level. Both approaches aim at restoration of an open reading frame, leading to expression of a partially function, BMD-like dystrophin. Gene replacement trials using modified adeno-associated viruses (AAVs) have been reported (Muzyczka, Curr Top Microbiol Immunol 158, 97-129 (1992); Carter, Mol Ther 10, 981-989 (2004); Samulski et al., Annu Rev Virol 1, 427-451 (2014)), but transgene packaging capacity of AAV is limited to ˜5 kb. Because the DMD cDNA is 11.4 kb, current viral vectors make use of one of several internally-deleted but in-frame microdystrophin CDNAs (Duan, Mol Ther 26, 2337-2356 (2018)). An alternate approach is to restore the mRNA reading frame by delivering an antisense sequence that binds to key exon definition elements in the pre-mRNA, inhibiting the recognition of a specific exon by the spliceosome, leading to exclusion of the target exon from the mature RNA. Such antisense sequences can consist of antisense oligonucleotides (AONs), or phoshphorodiamidate morpholino oligomers (PMO), such as eteplirsen, the first such therapy approved by the FDA for treatment of DMD due to mutations amenable to skipping of exon 51 (Barthelemy et al., Neuromuscul Disord 28, 803-824 (2018); Wein et al., Pediatr Clin North Am 62, 723-742 (2015); Alfano et al., Medicine (Baltimore) 98, e15858 (2019)).

Despite many lines of research following the identification of the DMD gene, treatment options are limited. There thus remains a need in the art for treatments for MDs, including DMD and BMD, including treatments for one or more mutations of the DMD gene.

### SUMMARY

The disclosure provides products and methods for preventing disease, delaying the progression of disease, and/or treating disease in patients with one or more 5′ mutations of the DMD gene. More particularly, the disclosure provides products and methods using a U7snRNA approach to induce skipping of DMD exon 2, with a particular goal of restoring wild-type dystrophin rather than an internally-deleted BMD-like dystrophin.

The disclosure provides a nucleic acid comprising a nucleotide sequence selected from the group consisting of: a nucleotide sequence comprising at least 80% identity to the sequence set forth in SEQ ID NO: 1; a nucleotide sequence complementary to the nucleotide sequence comprising at least 80% identity to the sequence set forth SEQ ID NO: 1; a nucleotide sequence comprising the sequence set forth in SEQ ID NO: 1; and a nucleotide sequence complementary to the nucleotide sequence comprising the sequence set forth in SEQ ID NO: 1.

The disclosure provides a recombinant adeno-virus associated (rAAV) comprising such nucleic acid. In some aspects, the rAAV is rAAV1, rAAV2, rAAV3, rAAV4, rAAV5, rAAV6, rAAV7, rAAV8, rAAV9, rAAV10, rAAV11, rAAV12, rAAV13, rAAV-anc80, rAAV rh.74, rAAV rh.8, rAAVrh. 10, or rAAV-B1. In some aspects, the rAAV is rAAV9. In some aspects, the rAAV is self-complementary.

The disclosure provides a composition comprising such a nucleic acid or rAAV and a carrier, diluent, excipient, and/or adjuvant.

The disclosure provides a method of treating, preventing or ameliorating a muscular dystrophy in a subject in need thereof comprising the step of administering to the subject such an rAAV or a composition comprising the rAAV, wherein the rAAV is administered at a dose of about 1.0×10¹⁰ vg/kg to about 1.0×10¹⁶ vg/kg. In some aspects, the dose is about 1.0×10¹¹ vg/kg to about 1.0×10¹⁵ vg/kg. In some aspects, the dose is about 1.0×10¹³ vg/kg to about 5.0×10¹³ vg/kg. In some aspects, the dose is about 2.0×10¹³ vg/kg to about 4.0×10¹³ vg/kg. In some aspects, the dose is about 3.0×10¹³ vg/kg.

In some aspects, the method comprises administering an initial dose followed by a second greater dose. In some aspects, the initial dose is about 1.0×10¹³ vg/kg to about 5.0×10¹³ vg/kg. In some aspects, the second dose is about 6.0×10¹³ vg/kg to about 4.0×10¹⁴ vg/kg. In some aspects, the initial dose is about 2.0×10¹³ vg/kg to about 4.0×10¹³ vg/kg. In some aspects, the initial dose is about 3.0×10¹³ vg/kg. In some aspects, the second dose is about 7.0×10¹³ vg/kg to about 9.0×10¹³ vg/kg. In some aspects, the second dose is about 8.0×10¹³ vg/kg.

In some aspects, the rAAV is administered via a systemic route. In some aspects, the systemic route is by injection, infusion or implantation. In some aspects, the systemic route is an intravenous route. In some aspects, the rAAV is administered by infusion over approximately one hour.

The disclosure provides products, methods, and uses for treating muscular dystrophy. In some aspects, the muscular dystrophy is Duchenne Muscular Dystrophy or Becker Muscular Dystrophy.

In some aspects of the methods and uses of the disclosure, the level of functional dystrophin gene expression or protein expression in a cell or tissue of the subject is increased after administration of the rAAV as compared to the level of functional dystrophin gene expression or protein expression before administration of the rAAV. In some aspects, the expression of functional dystrophin in the cell is detected by measuring the dystrophin protein level by Western blot, immunofluorescence, or immunohistochemistry in muscle biopsied before and after administration of the rAAV.

In some aspects of the methods and uses of the disclosure, the level of serum creatinine kinase in the subject is decreased after administration of the rAAV as compared to the level of serum creatinine kinase before administration of the rAAV.

In some aspects, the methods and uses of the disclosure result in improved muscle strength, improved muscle function, improved mobility, improved stamina, or a combination of two or more thereof in the subject.

In some aspects, the disclosure provides products, methods and uses, wherein muscular dystrophy progression in the subject is delayed or wherein muscle function in the subject is improved after administration of the rAAV. In some aspects, this delay in progression or improvement in muscle function is measured by the six minute walk test, time to rise test, ascend 4 steps test, ascend and descend 4 steps test, North Star Ambulatory Assessment (NSAA), the forced vital capacity (FVC) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

In some aspects, the methods or uses of the disclosure further comprise a second or combination therapy or administering a second or combination therapy. In some aspects, the methods or uses of the disclosure further comprise a glucocorticoid or administering a glucocorticoid.

The disclosure provides a method of expressing a dystrophin gene in a cell of a subject comprising administering to the subject a nucleic acid comprising a nucleotide sequence selected from the group consisting of: a nucleotide sequence comprising at least 80% identity to the sequence set forth in SEQ ID NO: 1; a nucleotide sequence complementary to the nucleotide sequence comprising at least 80% identity to the sequence set forth SEQ ID NO: 1; a nucleotide sequence comprising the sequence set forth in SEQ ID NO: 1; and a nucleotide sequence complementary to the nucleotide sequence comprising the sequence set forth in SEQ ID NO: 1.

The disclosure provides a method of expressing a dystrophin gene in a cell of a subject comprising administering to the subject an rAAV or a composition comprising an rAAV comprising a nucleic acid comprising a nucleotide sequence selected from the group consisting of: a nucleotide sequence comprising at least 80% identity to the sequence set forth in SEQ ID NO: 1; a nucleotide sequence complementary to the nucleotide sequence comprising at least 80% identity to the sequence set forth SEQ ID NO: 1; a nucleotide sequence comprising the sequence set forth in SEQ ID NO: 1; and a nucleotide sequence complementary to the nucleotide sequence comprising the sequence set forth in SEQ ID NO: 1, wherein the rAAV is administered to the subject at a dose of about 1.0×10¹⁰ vg/kg to about 1.0×10¹⁶ vg/kg.

The disclosure provides the use of an rAAV comprising a nucleic acid comprising a nucleotide sequence selected from the group consisting of: a nucleotide sequence comprising at least 80% identity to the sequence set forth in SEQ ID NO: 1; a nucleotide sequence complementary to the nucleotide sequence comprising at least 80% identity to the sequence set forth SEQ ID NO: 1; a nucleotide sequence comprising the sequence set forth in SEQ ID NO: 1; and a nucleotide sequence complementary to the nucleotide sequence comprising the sequence set forth in SEQ ID NO: 1, wherein the rAAV is at a dose of about 1.0×10¹⁰ vg/kg to about 1.0×10¹⁶ vg/kg.

In some aspects, the dose of rAAV is about 1.0×10¹¹ vg/kg to about 1.0×10¹⁵ vg/kg. In some aspects, the dose is about 1.0×10¹³ vg/kg to about 5.0×10¹³ vg/kg. In some aspects, the dose is about 2.0×10¹³ vg/kg to about 4.0×10¹³ vg/kg. In some aspects, the dose is about 3.0×10¹³ vg/kg.

In some aspects, the rAAV is administered or formulated at an initial dose and is followed by a second greater dose. In some aspects, the initial dose is about 1.0×10¹³ vg/kg to about 5.0×10¹³ vg/kg. In some aspects, the initial dose is about 2.0×10¹³ vg/kg to about 4.0×10¹³ vg/kg. In some aspects, the initial dose is about 3.0×10¹³ vg/kg. In some aspects, the second dose is about 6.0×10¹³ vg/kg to about 4.0×10¹⁴ vg/kg. In some aspects, the second dose is about 7.0×10¹³ vg/kg to about 9.0×10¹³ vg/kg. In some aspects, the second dose is about 8.0×10¹³ vg/kg.

In some aspects, the rAAV is administered or formulated for delivery via a systemic route. In some aspects, the systemic route is by injection, infusion or implantation. In some aspects, the systemic route is an intravenous route. In some aspects, the rAAV is administered by infusion over approximately one hour.

In some aspects, the muscular dystrophy is Duchenne Muscular Dystrophy or Becker Muscular Dystrophy.

In some aspects of the methods and uses of the disclosure, the level of functional dystrophin gene expression or protein expression in a cell of the subject is increased after administration of the rAAV as compared to the level of functional dystrophin gene expression or protein expression before administration of the rAAV. In some aspects, the level of expression of functional dystrophin in the cell is detected by measuring the dystrophin protein level by Western blot, immunofluorescence, or immunohistochemistry in muscle biopsied before and after administration of the rAAV.

In some aspects of the methods and uses of the disclosure, the level of serum creatinine kinase is decreased in the subject after administration of the rAAV as compared to the level of serum creatinine kinase before administration of the rAAV.

The disclosure provides products, methods and uses which result in improved muscle strength, improved muscle function, improved mobility, improved stamina, or a combination of two or more thereof in the subject.

The disclosure provides products, methods and uses wherein muscular dystrophy progression in the subject is delayed or wherein muscle function in the subject is improved after administration of the rAAV as measured by the six minute walk test, time to rise test, ascend 4 steps test, ascend and descend 4 steps test, North Star Ambulatory Assessment (NSAA), the forced vital capacity (FVC) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

In some aspects, the disclosure provides methods and uses further comprising administering a second or combination therapy. In some aspects, such methods and uses further comprise administering a glucocorticoid.

In some aspects, the disclosure provides methods and uses wherein expression of the dystrophin gene in the cell is measured in the subject by detecting greater than 1 rAAV vector genome copy per nucleus.

Other features and advantages of the disclosure will become apparent from the following description of the drawing and the detailed description. It should be understood, however, that the drawing, detailed description, and the examples, while indicating embodiments of the disclosed subject matter, are given by way of illustration only, because various changes and modifications within the spirit and scope of the disclosure will become apparent from the drawing, detailed description, and the examples.

### BRIEF DESCRIPTION OF THE DRAWINGS

FIGS. 1A-E show dystrophin restoration following systemic delivery of scAAV9.U7-ACCA in adult Dup2 mice and efficient exon skipping. Mice were injected via tail vein at 2 months of age with scAAV9.U7-ACCA (7.6e13 vg/kg) and sacrificed 3 months later. Mice were also injected with 6α-methylprednisolone 21-hemisuccinate sodium salt (PDN) or an equivalent volume of saline. FIG. 1A shows representative reverse transcription polymerase chain reaction (RT-PCR) analysis of tibialis anterior (TA), gastrocnemius (Gas), triceps (Tri), heart (Hrt) and diaphragm (dia) RNA from treated Dup2 mice compared to TA from WT and non-treated Dup2 mice. Primers are located in exon 1 and exon 3, resulting in three amplicons that contained either two copies (Dup2; 340 base pairs (bp)), one copy (WT; 278 bp), or no copies (Del2; 216 bp) of exon 2. FIG. 1B shows quantification of RT-PCR transcripts (obtained as in panel A) showing robust skipping that results in two therapeutic transcripts (WT in blue, and Del2 in violet) in skeletal and cardiac muscles. Data are represented as means±SD from Dup2 mice treated with either PDN alone (N=5, except for the TA muscle where N=4); the ACCA vector alone (N=5); or ACCA plus PDN (N=6). FIG. 1C shows immunostaining for dystrophin expression in muscles from a WT control, untreated Dup2, and treated Dup2 mice. Scale bars, 100 μm. FIG. 1D shows representative images from hematoxylin and eosin (H&E) staining of TA and diaphragm from WT, untreated, and treated Dup2 mice showing diminished centronucleation and fiber size variation in ACCA-treated animals. FIG. 1E shows quantification of centronucleation of myofibers from TA and diaphragm. Due to sample size, normality was tested using a Shapiro-Wilk test and significance was tested using a one-way ANOVA. For TA: Dup2 versus WT, ****P=0.0001; Dup2 versus Dup2+PDN, non-significant P=0.3688; Dup2 versus Dup2+ACCA alone, *P=0.0135, or with ACCA+PDN, ****P=0.0001. For Diaphragm: Dup2 versus WT, ****P=0.0001; Dup2 versus Dup2+PDN, non-significant P=0.5699; Dup2 versus Dup2+ACCA alone, ****P=0.0001, or with ACCA+PDN, ****P=0.0001. Data are represented as means±SD of the percentage of centronucleated fibers.

FIGS. 2A-G show that the restoration of dystrophin following systemic delivery of scAAV9.U7-ACCA in adult Dup2 mice results in sarcolemmal localization of dystrophin binding partners and improves muscle pathology, membrane integrity, and contraction-induced damage. FIG. 2A shows immunostaining of β-sarcoglycan (β-SG), β-dystroglycan (β-DG) and neuronal nitric oxidase synthetase (nNOS) in tibialis anterior of treated Dup2 mice three months post-injection. Results were obtained from WT, Dup2 injected with saline, glucocorticoid (PDN), scAAV9.U7-ACCA alone or scAAV9.U7-ACCA combined with PDN. Scale bars, 100 μm. N=6 for the PDN only treated mice; N=5-6 for ACCA and N=5-6 for ACCA+PDN. FIG. 2B shows percent of positive Evans blue dye (EBD) fibers following exhaustive treadmill exercise. Results were obtained from tibialis anterior (TA); gastrocnemius (Gas); triceps (Tri), and diaphragm (Dia) from Dup2 mice injected with saline, PDN alone, scAAV9.U7-ACCA alone, or scAAV9.U7-ACCA combined with PDN. N=4 for all groups. Three different sections and a minimum of 400 fibers were counted for each mouse. Data are represented as means±SD of the percent of EBD positive fiber. FIG. 2C shows absolute and FIG. 2D shows normalized specific force following tetanic contraction. Due to sample size, normality was tested using a Shapiro-Wilk test and significance was tested using a one-way ANOVA. Dup2 versus WT, **P=0.0039; Dup2 versus Dup2+PDN, non-significant P=0.3441; Dup2 versus Dup2+ACCA alone, ***P=0.0001, or with ACCA+PDN, ***P=0.0007 for FIG. 2C. Dup2 versus WT, ****P=0.0001; Dup2 versus Dup2+PDN, **P=0.0078; Dup2 versus Dup2+ACCA alone, **** P=0.0001, or with ACCA plus PDN, ****P=0.0001 for FIG. 2D. Data are represented as means±SD of the force generated. FIG. 2E shows loss of force following repetitive eccentric contractions. Two-way analysis of variance (ANOVA) of WT versus Dup2, or treated Dup2 versus untreated Dup2, *P<0.05 and ****P<0.001. Bonferroni's post hoc analysis for contractions 2-10. In FIGS. 2C-D, N=5-10 animals studied for each condition; error bars are s.d.; box boundaries mark the first and third quartiles. In FIG. 2E, N=5-10 muscles from at least 4 animals, error bars are standard error of the mean (SEM). FIG. 2F shows the cumulative ribosome footprint (RPF) coverage from scAAV9.U7-ACCA treated (red) and untreated (blue) mouse Dup2 tibialis (TA) muscle. The arrow indicates the location of the 5′ end of the Dp71 transcript. FIG. 2G shows Dup2 mouse genome-wide levels of RPF-Seq reads from scAAV9.U7-ACCA treated versus untreated TA muscle.

FIGS. 3A-E show early systemic delivery of scAAV9.U7-ACCA in young Dup2 mice allowing long lasting efficient exon 2 skipping and dystrophin expression. Mice were injected via facial vein at P0-P1 days of age with scAAV9.U7-ACCA (1.8e14 vg/kg) and sacrificed 6 months later. FIG. 3A shows RT-PCR analysis of RNA from TA, Gas, Tri, Hrt and Dia from Dup2 mice compare to a TA from WT and non-treated Dup2 mice using primers located in exon 1 and exon 3. Amplicon sizes are as follows: Dup2=340 bp; WT=278 bp; and Del2=216 bp. FIG. 3B shows RT-PCR quantification of the Dup2 (yellow), WT (blue), and Del2 (violet) transcripts, represented as means±SD from N=9 mice. FIG. 3C shows representative immunofluorescence of dystrophin in muscles from WT control, untreated and treated Dup2 mice. Scale bars, 100 μm. FIG. 3D shows representative images from H&E-stained TA, Gas, Tri, Hrt and Dia muscles from WT control, untreated and treated Dup2 mice. FIG. 3E shows quantification of centronucleation from treated and untreated TA and diaphragm. Due to the sample size, normality was tested using a Shapiro-Wilk test and significance was tested using a Mann-Whitney test. For TA: Dup2 versus WT, **P=0.0062; Dup2 versus Dup2+ACCA, NS=0.0655. For Diaphragm: Dup2 versus WT, ****P=0.0001; Dup2 versus Dup2+ACCA, ****P=0.0001. Data are represented as means±SD of the percent of centronucleated fiber. N=3-4 for control mice (Dup2 and WT); N=9 for treated mice. Tibialis anterior (TA); Gastrocnemius (Gas); Triceps (Tri); Heart (Hrt); Diaphragm (Dia).

FIGS. 4A-G show early systemic delivery of scAAV9.U7-ACCA in young Dup2 mice prevents muscle pathology at least 6 months after injection. FIG. 4A shows absolute and FIG. 4B shows normalized specific force following tetanic contraction in tibialis anterior 3 months after injection. Due to sample size, normality was tested using a Shapiro-Wilk test, and significance was tested using a one-way ANOVA. Dup2 (yellow, N=10) versus WT (blue, N=7), ****P=0.001; Dup2 versus Dup2+ACCA (violet, N=8), P=non-significant for FIG. 4A. Dup2 (N=10; blue) versus WT, ****P=0.0001; Dup2 versus Dup2+ACCA, ***P=0.0001 for FIG. 4B. Data are represented as means±SD of the force generated. FIG. 4C shows loss of force following repetitive eccentric contractions. Two-way analysis of variance (ANOVA) of WT versus Dup2, or Dup2+ACCA versus Dup2, ****P<0.001. Bonferroni's post hoc analysis for contractions 2-10. FIG. 4D shows absolute and FIG. 4E shows normalized specific force following tetanic contraction 6 months after injection. Due sample size a normality was tested using a Shapiro-Wilk test and significance was tested using a one-way ANOVA. Dup2 (yellow, N=9) versus WT (blue, N=9), ****P=0.0001; Dup2 versus Dup2+ACCA (violet, N=6), ****P=0.0001 for FIG. 4D. Dup2 versus WT, ****P=0.0001; Dup2 versus Dup2+ACCA, ****P=0.0001 for FIG. 4E. Data are represented as means±SD of the force generated. FIG. 4F shows loss of force following repetitive eccentric contractions. Two-way analysis of variance (ANOVA) of WT versus Dup2, or Dup2+ACCA versus Dup2, ****P<0.001. Error bars are SEM for e and h. Bonferroni's post hoc analysis for contractions 2-10. FIG. 4G shows the number of viral genomes as measured by quantitative PCR in different muscles 3 and 6 months after injection. Results are represented as means±SD. N=5. There were no statistical differences between 3 and 6 months using a two-way ANOVA.

FIGS. 5A-B show efficient in-frame and out of frame exon skipping using scAAV1.U7-ACCA in patient cells harboring mutations within exons 1-4 results in dystrophin expression. Five different patient fibroblasts cell lines were transdifferentiated into myoblasts (referred as FibroMyoD) and transduced with scAAV1.U7-ACCA (1.5e11 vg total). FIG. 5A shows RT-PCR results that were obtained 3 days post infection. On the left of each gel image are the exon splicing outcomes resulting in each amplicon (as confirmed by sequencing). Results were obtained either following PDN alone, scAAV1.U7-ACCA alone, or scAAV1.U7-ACCA combined with PDN. FIG. 5B shows Western blot of dystrophin following of FibroMyoD cell lines for 14 days. Myosin heavy chain (MHC) was used as a loading control.

FIGS. 6A-D show increased amount of dystrophin following treatment three months after injection. FIG. 6A shows representative Western blot of dystrophin obtained from muscles of treated adult mice. Results were obtained either following glucocorticoid treatment (PDN) alone, scAAV9.U7-ACCA alone, or scAAV9.U7-ACCA combined with PDN. Arrow points out full-length dystrophin protein. FIG. 5B shows Western blot quantification of dystrophin restoration. Data are represented as means±SD and as a percent of WT dystrophin expression (blue bar). N=5-6 for the PDN only treated mice (yellow bar); N=5-7 for ACCA (green bar); and N=5-7 for ACCA+PDN (violet bar). FIG. 6C shows qPCR quantification of the number of viral genomes. Results are represented as means±SD (N=5-7 per group). There were no statistical differences between scAAV9.U7-ACCA alone and scAAV9.U7-ACCA in combination with PDN except in the diaphragm. FIG. 6D shows Western blot quantification of utrophin in the treated mice (N=5-7 per group), with α-actinin was used as a loading control. Results are represented as a ratio of utrophin to α-actinin. There were no statistical differences between PDN alone, scAAV9.U7-ACCA alone, and scAAV9.U7-ACCA plus PDN within each muscle group. Tibialis anterior (TA); Gastrocnemius (Gas); Triceps (Tri); Heart (Hrt); Diaphragm (Dia).

FIGS. 7A-C show restoration of dystrophin following systemic delivery of scAAV9.U7-ACCA in adult Dup2 mice allows sarcolemmal localization of dystrophin binding partners three months post-injection in different muscles. Immunostaining of β-sarcoglycan (β-SG) (FIG. 7A), β-dystroglycan (β-DG) (FIG. 7B), and neuronal nitric oxidase synthetase (nNOS) (FIG. 7C) was carried out in triceps (Tri) and diaphragm (Dia) of treated Dup2 mice. Results were obtained from WT, and from Dup2 mice injected with either saline or glucocorticoid (PDN) alone, scAAV9.U7-ACCA alone, or scAAV9.U7-ACCA combined with PDN. Scale bars, 100 μm. N=6 for the PDN only treated mice; N=5-6 for ACCA and N=5-6 for ACCA+PDN.

FIGS. 8A-B show dystrophin expression following scAAV9.U7-ACCA treatment in adult Dup2 mice at 1, 3 and 6 months post-injection. FIG. 8A shows representative Western blot of dystrophin obtained in tibialis anterior (TA), gastrocnemius (Gas), triceps (Tri), heart (Hrt), and diaphragm (Dia) from treated mice compared to tibialis anterior from a WT or a non-treated Dup2 mouse. Alpha (α)-actinin was used as a loading control. Arrow points out full-length dystrophin protein (FIG. 8A). FIG. 8B shows Western blot quantification of dystrophin expression. Data are represented as means±SD and as a percent of WT dystrophin expression. N=3-5 for control mice (Dup2 and WT); and N=5-9 for treated mice at each time point.

FIGS. 9A-E show efficient exon skipping following scAAV9.U7-ACCA treatment in young Dup2 mice 1 month post-injection. Mice were injected via facial vein at P0-1 day of age with scAAV9.U7-ACCA (1.8e14 vg/kg) and sacrificed 1 month later. FIG. 9A shows RT-PCR analysis from tibialis anterior (TA), gastrocnemius (Gas), triceps (Tri), heart (Hrt), and diaphragm (Dia) in Dup2 treated mice compare to TA of WT and non-treated mice. FIG. 9B shows quantification of RT-PCR transcripts (obtained as in panel A) showing robust skipping that results in two therapeutic transcripts (WT in blue, and Del2 in violet) in skeletal and cardiac muscles. RT-PCR quantification of the Dup2, WT and del2 transcript. N=4-5 for the one month treated mice. Data are represented as means±SD. FIG. 9C shows immunofluorescence of dystrophin in several muscles from WT control, untreated and treated Dup2, 1 month post-injection. Scale bars, 100 μm. FIG. 9D shows representative images from hematoxylin and eosin staining of TA and Gas, Tri, Hrt and Dia from WT control, untreated and treated Dup2. FIG. 9E shows quantification of centronucleation from treated and untreated TA and Diaphragm. Due sample size, a normality was tested using a Shapiro-Wilk test and significance was tested using a Mann-Whitney test. For TA: Dup2 versus WT, *P=0.0171; Dup2 versus Dup2+ACCA, NS=0.6346. For Diaphragm: Dup2 versus WT, ****P=0.0001; Dup2 versus Dup2+ACCA, ****P=0.0001. Data are represented as means±SD of the percent of centronucleated fiber. N=3-4 for control mice (Dup2 and WT); N=2-5 for treated mice. Tibialis anterior (TA); Gastrocnemius (Gas); Triceps (Tri); Heart (Hrt); Diaphragm (Dia).

FIGS. 10A-E show exon skipping following scAAV9.U7-ACCA treatment in young Dup2 mice 3 months post-injection. Mice were injected via facial vein at P0-1 day of age with scAAV9.U7-ACCA (1.8e14 vg/kg) and sacrificed 3 months later. FIG. 10A shows RT-PCR analysis from tibialis anterior (TA), gastrocnemius (Gas), triceps (Tri), heart (Hrt) and diaphragm (Dia) in Dup2 treated mice compare to TA of WT and non-treated mice. FIG. 10B shows quantification of RT-PCR transcripts (obtained as in FIG. 10A) showing robust skipping that results in two therapeutic transcripts (WT in blue, and Del2 in violet) in skeletal and cardiac muscles. N=7 for three months treated mice. Data are represented as means±SD. FIG. 10C shows immunofluorescence of dystrophin in several muscles from WT control, untreated and treated Dup2. Scale bars, 100 μm. FIG. 10D shows representative images from hematoxylin and eosin staining of TA and Gas, Tri, Hrt and Dia from WT control, untreated and treated Dup2. FIG. 10E shows quantification of centronucleation in tibialis anterior and diaphragm from treated and untreated mice. Due to sample size, a normality was tested using a Shapiro-Wilk test and significance was tested using either an unpaired t-test or a Mann-Whitney test. For TA: Dup2 versus WT, ****P=0.0001; Dup2 versus Dup2+ACCA, ****P=0.0001. For Diaphragm: Dup2 versus WT, **P=0.002; Dup2 versus Dup2+ACCA, NS=0.01665. Data are represented as means±SD of the percent of centronucleated fiber. N=3-4 for control mice (Dup2 and WT); N=7 for treated mice. Tibialis anterior (TA); Gastrocnemius (Gas); Triceps (Tri); Heart (Hrt); Diaphragm (Dia).

FIG. 11 shows histopathology of several organs demonstrating absence of pathology signs 6 months following systemic injection. Representative pictures of several internal organs stained by H&E from control (WT), untreated, and treated 6 month-old Dup2 mice (N=5). In treated mice, occasional small patchy foci of inflammatory cells, including neutrophils, were found inconsistently in livers. The other tissues are unremarkable.

FIGS. 12A-B shows RT-PCR analysis following intramuscular (TA) scAAV9.U7.ACCA dose escalation studies in Dup2 mice shows increasing efficiency of exon 2 skipping, resulting in therapeutic transcripts (both WT and A2). Mice were injected once at 8/9 weeks old and sacrificed 4-5 weeks post-injection. Representative images (N=4 TAs/dose) of DMD RT-PCR are shown in FIG. 12A and subsequent quantification is reported as the mean % of total transcript of each species (N≥8 TAs/dose) in FIG. 12B.

FIGS. 13 shows immunofluorescence staining of dystrophin with a C-terminal antibody showing abundant expression 4-5 weeks after a single intramuscular (TA) injection. Representative 10× images (N=3 muscles/dose) of protein expression showed that skipping of either one (WT) or both (Δ2) copies of exon 2 results in significant dystrophin expression, regardless of isoform.

FIGS. 14A-B shows immunofluorescence staining of dystrophin with a C-terminal antibody showing abundant expression 4-5 weeks after a single intramuscular (TA) injection. Representative 10× images (N=3/dose) of protein expression (FIG. 14A) shows that skipping of either one (WT) or both (42) copies of exon 2 resulted in significant dystrophin expression (FIG. 14B), regardless of isoform (*0.5<P<0.005; **0.05<P<0.005; ***0.005<P<0.0005; ****0.0005<P). Data were tested for normality using the D′Agostino-Pearson and Shapiro-Wilk normality test. If normal, a one-way ANOVA analysis was conducted. If not normal, a two-tailed Kruskal-Wallis test was used to analyze absolute and specific force. A two-way ANOVA test was used to analyze eccentric contraction.

FIGS. 15A-C shows results of physiology testing conducted on 12-13 week-old Dup2 mice following a single intramuscular (TA) injection at 8-9 weeks of age. A nearly complete rescue of absolute force was seen (FIG. 15A), while a partial rescue of specific force (FIG. 15B) and force drop following repeated contractions (FIG. 15C) was seen in the injected Dup2 mice compared to un-injected Dup2 controls. Quantification of absolute and specific force are reported as the mean and standard deviation (s.d.). Eccentric contraction values are reported as mean and standard error of the mean (s.e.m.) (*0.5<P<0.005; **0.05<P<0.005; ***0.005<P<0.0005;****0.0005<P). Data was tested for normality using the D'Agostino-Pearson and Shapiro-Wilk normality test. If normal, a one-way ANOVA analysis was conducted. If not normal, a two-tailed Kruskal-Wallis test was used to analyze absolute and specific force. A two-way ANOVA test was used to analyze eccentric contraction.

FIGS. 16A-B shows images of RT-PCR (FIG. 16A) and transcript quantification (FIG. 16B) of dystrophin expression after injection with various doses of scAAV9.U7ACCA. TA, Gas, Triceps, Diaphragm, and Heart from Dup2 mice treated at 8-9 weeks of age were examined at 4 weeks post-injection for the amount of each transcript including two (Dup2, 340 bp), one (WT, 278 bp), or zero (Δ2, 216 bp) copies of exon 2. Six doses (1.8 E12, 5.8 E12, 1.8 E13, 5.8 E13, 1.8 E14 or 4.7 E14 vg/kg) of scAAV9.U7ACCA were examined for exon 2 skipping and showed a dose-related response. Images of the dmd RT-PCR (FIG. 16A, N=3/dose) and subsequent quantification is reported as the mean % total transcript of each species (FIG. 16B, N=3/dose).

FIG. 17 shows representative images of dystrophin staining after injection of various doses scAAV9.U7.ACCA. Dup2 mice treated at 1 month of age with either 5.8 E13, 1.8 E14 or 4.7 E14 vg/kg scAAV9.U7.ACCA (n=3 each dose). Four weeks post-injection TA, Gas, Triceps, Diaphragm, and Heart were stained to show amount of properly localized dystrophin (red staining).

FIGS. 18A-B shows dystrophin expression and quantification after injection with various doses of scAAV9.U7.ACCA. Dup2 mice were treated for 4 weeks with either 5.8 E13, 1.8 E14 or 4.7 E14 vg/kg scAAV9.U7.ACCA. (A) Representative images (N=2/dose) of immunoblots (FIG. 18A) show increase in dystrophin protein (red bands) up to the highest dose and dystrophin quantification (FIG. 18B) shows nearly wild type levels in the heart and supranormal levels in the triceps. Quantification is reported as the mean and s.d. of at least two muscles per dose (*0.5<P<0.005; **0.05<P<0.005. Data were tested for normality using the D'Agostino-Pearson and Shapiro-Wilk normality test. If normal, a one-way ANOVA analysis was conducted. If not normal, a two-tailed Kruskal-Wallis test was used for analysis).

FIGS. 19A-B shows dystrophin expression and quantification after injection with various doses of scAAV9.U7.ACCA. TA, Gas, Triceps, Diaphragm, and Heart from Dup2 mice treated at 8-9 weeks of age were examined at 12 weeks post-injection for the amount of each transcript including two (Dup2, 340 bp), one (WT, 278 bp), or zero (42, 216 bp) copies of exon 2. Three doses (5.8 E13, 1.8 E14 or 4.7 E14 vg/kg) of scAAV9.U7ACCA were examined for exon 2 skipping and showed a dose-related response. Representative images of the dmd RT-PCR (FIG. 19A, n=5/dose) and subsequent quantification is reported as the mean % total transcript of each species (FIG. 19B, n≥5/dose).

FIGS. 20 shows dystrophin expression in Dup2 mice after treatment with various doses of scAAV9.U7.ACCA. Dup2 mice were treated at 1 month of age with either 5.8 E13, 1.8 E14 or 4.7 E14 vg/kg scAAV9.U7.ACCA (N=3/dose). Twelve weeks post-injection TA, Gas, Triceps, Diaphragm, and Heart were stained to show amount of properly localized dystrophin (red staining).

FIGS. 21A-B shows dystrophin expression in various muscles of Dup2 mice using immunoblot analysis after treating with various doses of scAAV9.U7.ACCA. Dup2 mice were treated at 1 month with either 5.8 E13, 1.8 E14 or 4.7 E14 vg/kg scAAV9.U7.ACCA. Twelve weeks post-injection immunoblot analysis was performed in TA, Gas, Triceps, Diaphragm, and Heart. Representative images of dystrophin (n=5/dose) for each dose and muscle are shown in FIG. 21A. In each muscle, dystrophin expression (red bands) increased with the dose and reached its maximum at 4.7 E14 vg/kg dose with levels comparable to WT in the triceps, diaphragm and heart. Quantification (FIG. 21B) is reported as the mean and s.d. of at least three muscles per dose (*0.5<P<0.005; ** 0.05<P<0.005. Data were tested for normality using the D'Agostino-Pearson and Shapiro-Wilk normality test. If normal, a one-way ANOVA analysis was conducted. If not normal, a two-tailed Kruskal-Wallis test was used for analysis).

FIGS. 22A-C shows results of electrophysiology studies on TA muscle of Dup2 mice after administering various intravenous doses. Dup2 mice were treated at 1 month with the three highest doses (i.e., 5.8 E13, 1.8 E14 or 4.7 E14 vg/kg) of scAAV9.U7.ACCA. TA physiology was performed on 20-21 week old mice that show a dose-response curve and significant correction in absolute force in treated animals in comparison to WT controls (FIG. 22A). In contrast, there was only a partial improvement in specific fore (FIG. 22B) and force drop following repeated eccentric contraction (FIG. 22C). Quantification of absolute force and specific force is reported as the mean and s.d. Eccentric contraction (ECC) values are reported as mean and s.e.m. (*0.5<P<0.005; **0.05<P<0.005; ***0.005<P<0.0005; ****0.0005<P). Data were tested for normality using the D′Agostino-Pearson and Shapiro-Wilk normality test. If normal, a One-Way ANOVA analysis was conducted. If not normal, a two-tailed Kruskal-Wallis test was used to analyze absolute and specific force. A two-way ANOVA test was used to analyze eccentric contraction.

FIG. 23 shows percent dystrophin expression measured by Western blotting following systemic delivery of scAAV9.U7.ACCA alone or in combination with glucocorticoid. Dup2 mice were treated at 2 months with either 4.7E14 vg/kg scAAV9.U7.ACCA alone or in combination with PDN. Dystrophin protein expression in each of TA, Gas, Diaphragm (Dia), and Heart (Hrt) was quantified using Western blot analysis carried out 12-14 weeks post-injection.

FIGS. 24A-C show muscle strength/force following systemic delivery of scAAV9.U7.ACCA alone or in combination with glucocorticoid. Dup2 mice were treated at 2 months with either 4.7E14 vg/kg scAAV9.U7.ACCA alone or in combination with PDN (labeled as GC). TA force was measured 15-16 weeks post-injection (* 0.5<P<0.005; **0.05<P<0.005; ***0.005<P<0.0005; ****0.0005<P. Two-tailed Kruskal-Wallis tests were used to analyze absolute and specific force. 2 way ANOVA tests were used to analyze eccentric contraction).

FIG. 25 shows that treatment of human subjects is associated with mild transient elevation of transaminases and sustained decrease in serum creatinine kinase (CK). FIG. 25 shows levels of transaminases, alanine transaminase (ALT) and aspartate transaminase (AST) in both human subjects before treatment and through 90 days after treatment. FIG. 25 shows levels of serum CK in both human subjects before treatment and through 90 days after treatment. There was a 95% decrease in serum CK level from baseline (13,495 to 560 u/L) in U7-Dup2-01. There was an 81% decrease in serum CK level from baseline (5,103 to 947 u/L).

FIG. 26 shows the expression of apparently normal size dystrophin at 3 months post-injection in both subjects as demonstrated by the on-gel standard curve (normal muscle dilution series). Western blot was performed in duplicate and the mean is reported.

FIG. 27 shows exon skipping by RT-PCR demonstrated a biologic effect. At 3 months post-treatment, there was an increase in both WT and Del2 transcripts over baseline, and an increase in total therapeutic transcript in both subjects over baseline. Quantification of vector genomes in skeletal muscle 3 months post-treatment also is shown.

FIG. 28 shows that dystrophin expression was markedly improved in subject 1 at 3 months post-treatment over baseline.

### DETAILED DESCRIPTION

The products and methods described herein are used for preventing disease, delaying the progression of disease, and/or treating muscular dystrophies in patients with one or more 5′ mutations of the DMD gene. More particularly, the disclosure provides products and methods using a U7snRNA approach to induce skipping of DMD exon 2, with a particular goal of restoring wild-type dystrophin rather than an internally-deleted BMD-like dystrophin. Duplications of one or more exons cause up to 11% of all DMD cases, and duplications of exon 2 are the most common, accounting for 10% of all duplications (Bladen et al., Hum Mutat 36, 395-402 (2015); Flanigan et al., Neuromuscul Disord 19, 743-748 (2009)).

The disclosure provides products and methods to target exon 2 of the DMD gene. Targeting of exon 2 duplication mutations provides a very wide therapeutic window. For example, skipping of a single copy results in a full-length wild-type dystrophin, and skipping of both copies results in alternate translational initiation from a highly functional internal ribosome entry site (IRES) within exon 5 (Wein et al., Pediatr Clin North Am 62, 723-742 (2015)). Wild-type dystrophin is a 427 kD protein and the IRES-driven form of dystrophin is a 413 kD protein. Despite lacking the first calponin homology 1 (CH1) domain within N-terminal actin binding domain 1 (ABD1), the resulting protein is highly functional, as proven by the exceedingly mild phenotype in families who express this version of the protein due to a founder allele nonsense mutation within exon 1, many of whom have only myalgia and elevations in CK, and walk into their seventh decade or longer (Gurvich et al., Hum Mutat 30, 633-640 (2009)).

The disclosure provides antisense targeting sequences embedded into a modified U7 small nuclear RNA (U7snRNA) (Gorman et al., Proc Natl Acad Sci USA 95, 4929-4934 (1998)). This becomes a part of a small nuclear ribosomal protein complex (snRNP) that protects the antisense sequence from degradation and allows for accumulation in the nucleus where splicing occurs (Suter et al., Hum Mol Genet 8, 2415-2423 (1999)). The U7snRNA, which contains internal promoters allowing for continuous transcription of the downstream antisense sequences, in some aspects, is encapsidated into an AAV for widespread tissue delivery. This approach has been shown to be useful in vitro as well as in mouse and dog models of DMD (Wein et al., Nature Medicine 20, 992-1000 (2014); Goyenvalle et al., Hum Mol Genet 21, 2559-2571 (2012); Barbash et al., Gene Ther 20, 274-282 (2013); Bish et al., Mol Ther 20, 580-589 (2012); Vulin et al., Mol Ther 20, 2120-2133 (2012)). Thus, in some embodiments, the disclosure provides such U7snRNA for the prevention, treatment, or amelioration of diseases or disorders resulting from mutations of the DMD gene.

The disclosure provides a vector for delivering antisense targeting sequences. In some embodiments, the vector contains four copies of the U7snRNA in a self-complementary genome, with two copies targeting the splice acceptor site (sequence A) and two copies targeting the splice donor site (sequence C), encapsidated in AAV9 (scAAV9.U7-ACCA). The disclosure provides experimental results from an investigation of the efficacy of exon 2 skipping induced by this vector following systemic delivery in the Dup2 mouse (which carries the analogous mutation) (Vulin et al., Neuromuscul Disord 25, 827-834 (2015)), with or without co-treatment with prednisolone, as steroids are the only treatment shown to slow progression of DMD, and it has been shown previously that the DMD IRES is glucocorticoid responsive (Wein et al., Pediatr Clin North Am 62, 723-742 (2015)).

The disclosure provides experimental results showing that one-time tail vein injection of scAAV9.U7 into 2-month old Dup2 mice results in highly efficient exon skipping, translation of both full-length and IRES-driven dystrophin proteins, and correction of clinical, histopathological, and physiologic markers of disease. Similarly, a single neonatal injection of this vector results in highly efficient exon skipping, protein production, and almost complete reversion of the disease phenotype at 6 months. Such vectorized exon skipping holds significant clinical promise. Although antisense oligonucleotides or oligomers (AONs) approaches are promising, AON therapies require weekly (or similar) reinjections. In contrast, U7snRNA mediated skipping offers the advantage of continuous antisense sequence transcription and accumulation in the nucleus after a single injection. U7snRNA-mediated exon skipping has shown promising results in the golden retriever muscular dystrophy (GRMD) dog model (Kornegay, Skelet Muscle 7, 9 (2017)) in which injection of an AAV.U7 vector, targeted to exons 6 and 8, restored the reading frame, leading to significant expression of dystrophin and long-lasting improvements in muscle function (Bish et al., Mol Ther 20, 580-589 (2012)). Such studies demonstrated that even in large mammals, such AAV.U7 approaches are highly safe and efficient.

Additionally, the disclosure provides experimental results of investigating whether exon 2 skipping could lead to IRES activation in cells derived from patients with other mutations within exons 1-4, which account for ˜5-6% of DMD patients (https://www.dmd.nl), confirming efficient exon 2 skipping in the presence of other 5′ mutations, and leading to IRES-driven dystrophin expression. The experimental results provided herein support a therapeutic option for patients with mutations within exons 1-4. Thus, the disclosure in part provides products and methods, based on the identification of a glucocorticoid-inducible IRES in exon 5 of the DMD gene, the activation of which can generate a functional N-terminally truncated dystrophin isoform, and the use of exon 2-targeted antisense in a nucleic acid construct.

In some embodiments, the disclosure provides a nucleic acid comprising the U7-ACCA nucleotide sequence. The U7-ACCA nucleotide sequence is set out in SEQ ID NO: 1 below. The U7-ACCA is reverse complement, as it was inserted in the AAV backbone. This sequence does not contain the ITR and the probe and spacer.

SEQ ID NO: 1:
cacatacgcgtttcctaggaaaccagagaaggatcaaagcccctctcaca
caccggggagcggggaagagaactgttttgctttcattgtagaccagtga
aattgggaggggttttccgaccgaagtcagaaaacctgctccaaaaattt
agatgaaagagaagatcttcaaaagaaaacttgcggaagtgcgtctgtag
cgagccagggaaggacatcaactccactttcgatgagggtgagatcaagg
tgccatttccacacccctccactgatatgtgaatcacaaagcacagttcc
ttattcggttcgataaacaatattctaaaagactattaaaaccgctcgtt
tcttgagtttgtgaccgcttgtaaaggctatgcaaatgagtcagtgctga
ttggctgaaaacagccaatcacagctcctatgttgttatctagccacata
cgcgtttcctaggaaaccagagaaggatcaaagcccctctcacacaccgg
ggagcggggaagagaactgttttgctttcattgtagaccagtgaaattgg
gaggggttttccgaccgaagtcagaaaacctgctccaaaaattgcacaat
tttctaaggtaagaatttgcggaagtgcgtctgtagcgagccagggaagg
acatcaactccactttcgatgagggtgagatcaaggtgccatttccacac
ccctccactgatatgtgaatcacaaagcacagttccttattcggttcgat
aaacaatattctaaaagactattaaaaccgctcgtttcttgagtttgtga
ccgcttgtaaaggctatgcaaatgagtcagtgctgattggctgaaaacag
ccaatcacagctcctatgttgttatctagccacatacgcgtttcctagga
aaccagagaaggatcaaagcccctctcacacaccggggagcggggaagag
aactgttttgctttcattgtagaccagtgaaattgggaggggttttccga
ccgaagtcagaaaacctgctccaaaaattgcacaattttctaaggtaaga
atttgcggaagtgcgtctgtagcgagccagggaaggacatcaactccact
ttcgatgagggtgagatcaaggtgccatttccacacccctccactgatat
gtgaatcacaaagcacagttccttattcggttcgataaacaatattctaa
aagactattaaaaccgctcgtttcttgagtttgtgaccgcttgtaaaggc
tatgcaaatgagtcagtgctgattggctgaaaacagccaatcacagctcc
tatgttgttatctagccacatacgcgtttcctaggaaaccagagaaggat
caaagcccctctcacacaccggggagcggggaagagaactgttttgcttt
cattgtagaccagtgaaattgggaggggttttccgaccgaagtcagaaaa
cctgctccaaaaatttagatgaaagagaagatcttcaaaagaaaacttgc
ggaagtgcgtctgtagcgagccagggaaggacatcaactccactttcgat
gagggtgagatcaaggtgccatttccacacccctccactgatatgtgaat
cacaaagcacagttccttattcggttcgataaacaatattctaaaagact
attaaaaccgctcgtttcttgagtttgtgaccgcttgtaaaggctatgca
aatgagtcagtgctgattggctgaaaacagccaatcacagctcctatgtt
gtta

In some embodiments, the disclosure includes a nucleic acid comprising a nucleotide sequence that has at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identity to the sequence set forth in SEQ ID NO: 1. In some aspects, the disclosure includes a nucleic acid comprising a nucleotide sequence that is complementary to a nucleotide sequence that has at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identity to a sequence set forth in SEQ ID NO: 1. In some aspects, the disclosure includes a nucleotide sequence comprising the sequence set forth in SEQ ID NO: 1. In some aspects, the disclosure includes a nucleotide sequence complementary to the nucleotide sequence comprising the sequence set forth in SEQ ID NO: 1. In some aspects, the disclosure includes such nucleic acid under the control of a U7 promoter. In some aspects, the disclosure includes the delivery of such nucleic acid in a U7 small nuclear RNA (snRNA).

In some embodiments, the disclosure provides small nuclear ribonucleic acids (snRNAs), also commonly referred to as U-RNAs, to affect DMD expression. snRNAs are a class of small RNA molecules that are found within the splicing speckles and Cajal bodies of the cell nucleus in eukaryotic cells. Small nuclear RNAs are associated with a set of specific proteins, and the complexes are referred to as small nuclear ribonucleoproteins (snRNP, often pronounced “snurps”). Each snRNP particle is composed of a snRNA component and several snRNP-specific proteins (including Sm proteins, a family of nuclear proteins). The snRNAs, along with their associated proteins, form ribonucleoprotein complexes (snRNPs), which bind to specific sequences on the pre-mRNA substrate. They are transcribed by either RNA polymerase Il or RNA polymerase III. snRNAs are often divided into two classes based upon both common sequence features and associated protein factors, such as the RNA-binding LSm proteins. The first class, known as Sm-class snRNA, consists of U1, U2, U4, U4atac, U5, U7, U11, and U12. Sm-class snRNA are transcribed by RNA polymerase Il. The second class, known as Lsm-class snRNA, consists of U6 and U6atac. Lsm-class snRNAs are transcribed by RNA polymerase Ill and never leave the nucleus, in contrast to Sm-class snRNA. In some aspects, the disclosure includes the production and administration of an AAV vector comprising U7 snRNA for the delivery of DMD antisense sequences, such as the U7-ACCA nucleotide sequence set out in SEQ ID NO: 1 or a variant thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identity to the sequence set forth in SEQ ID NO: 1.

In some embodiments, the disclosure provides small nuclear ribonucleic acids (snRNAs), also commonly referred to as U-RNAs, to affect DMD expression. snRNAs are a class of small RNA molecules that are found within the splicing speckles and Cajal bodies of the cell nucleus in eukaryotic cells. Small nuclear RNAs are associated with a set of specific proteins, and the complexes are referred to as small nuclear ribonucleoproteins (snRNP, often pronounced “snurps”). Each snRNP particle is composed of a snRNA component and several snRNP-specific proteins (including Sm proteins, a family of nuclear proteins). The snRNAs, along with their associated proteins, form ribonucleoprotein complexes (snRNPs), which bind to specific sequences on the pre-mRNA substrate. They are transcribed by either RNA polymerase II or RNA polymerase III. snRNAs are often divided into two classes based upon both common sequence features and associated protein factors, such as the RNA-binding LSm proteins. The first class, known as Sm-class snRNA, consists of U1, U2, U4, U4atac, U5, U7, U11, and U12. Sm-class snRNA are transcribed by RNA polymerase Il. The second class, known as Lsm-class snRNA, consists of U6 and U6atac. Lsm-class snRNAs are transcribed by RNA polymerase Ill and never leave the nucleus, in contrast to Sm-class snRNA.

In some embodiments, the disclosure provides U7 snRNA molecules to interfere with DMD gene expression. U7 snRNA is normally involved in histone pre-mRNA 3′ end processing but, in some aspects, is converted into a versatile tool for splicing modulation or as antisense RNA that is continuously expressed in cells [Goyenvalle et al., Science 306(5702): 1796-9 (2004)]. Moreover, when embedded into a gene therapy vector, these small RNAs can be permanently expressed inside the target cell after a single injection [Levy et al., Eur. J. Hum. Genet. 18(9): 969-70 (2010); Wein et al., Hum. Mutat. 31(2): 136-42, (2010); Wein et al., Nat. Med. 20(9): 992-1000 (2014)]. The potential of U7snRNA systems in neuromuscular disorders using an AAV approach has been investigated in vivo (AAV.U7) [Levy et al., Eur. J. Hum. Genet. 18(9): 969-70 (2010); Wein et al., Hum. Mutat. 31(2): 136-42 (2010); Wein et al., Nat. Med. 20(9): 992-1000 (2014)]. A single injection of this AAV9.U7, targeting the defective RNA of a mouse model of Duchenne muscular dystrophy, results in long term correction of the disease in every muscle, including heart and diaphragm. The ability to target the heart is really important since DM1 patients display cardiac abnormalities.

In some embodiments, the disclosure utilizes AAV to deliver inhibitory U7snRNA to deliver a DMD antisense sequence, e.g., U7-ACCA, which binds to key exon definition elements in the pre-mRNA, inhibiting the recognition of a specific exon by the spliceosome, leading to exclusion of the target exon from the mature RNA, resulting in the expression of dystrophin. U7 snRNA is normally involved in histone pre-mRNA 3′ end processing but, in some aspects, is converted into a versatile tool for splicing modulation or as antisense RNA that is continuously expressed in cells [Goyenvalle et al., Science 306(5702): 1796-9 (2004)]. Moreover, when embedded into a gene therapy vector, these small RNAs can be permanently expressed inside the target cell after a single injection [Levy et al., Eur. J. Hum. Genet. 18(9): 969-70 (2010); Wein et al., Hum. Mutat. 31(2): 136-42, (2010); Wein et al., Nat. Med. 20(9): 992-1000 (2014)]. The potential of U7snRNA systems in neuromuscular disorders using an AAV approach has been investigated in vivo (AAV.U7) [Levy et al., Eur. J. Hum. Genet. 18(9): 969-70 (2010); Wein et al., Hum. Mutat. 31(2): 136-42 (2010); Wein et al., Nat. Med. 20(9): 992-1000 (2014)]. A single injection of this AAV9.U7, targeting the defective RNA of a mouse model of Duchenne muscular dystrophy, results in long term correction of the disease in every muscle, including heart and diaphragm. The ability to target the heart is really important since DM1 patients display cardiac abnormalities.

In some embodiments, the disclosure provides one or more copies of the nucleic acid is combined into a single vector. Thus, the disclosure includes a vector or vectors comprising a nucleic acid of the disclosure or a combination of nucleic acids of the disclosure. Such vectors include, for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule, to deliver one or more of the nucleic acids disclosed herein. In some aspects, the viral vector is an AAV. Thus, in some embodiments of the disclosure, U7snRNA is delivered via a viral vector, such as AAV.

In some aspects, the AAV lacks rep and cap genes. In some aspects, the AAV is a recombinant AAV (rAAV). In some aspects, the rAAV is a single-stranded AAV (ssAAV) or a self-complementary AAV (scAAV).

In some embodiments, the viral vector is an adeno-associated virus (AAV), such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), AAVrh.10 (i.e., an AAV containing AAVrh.10 ITRs and AAVrh.10 capsid proteins), AAV11 (i.e., an AAV containing AAV11 ITRs and AAV11 capsid proteins), AAV12 (i.e., an AAV containing AAV12 ITRs and AAV12 capsid proteins), AAV13 (i.e., an AAV containing AAV13 ITRs and AAV13 capsid proteins), AAV-anc80, AAV rh.74, AAV rh.8, AAVrh. 10, or AAV-B1.

AAV is a replication-deficient parvovirus, the single-stranded DNA genome of which is about 4.7 kb in length including 145 nucleotide inverted terminal repeat (ITRs). There are multiple serotypes of AAV, for example, as set out herein above. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. In some aspects, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

In some embodiments, DNA plasmids of the disclosure comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, AAV rh.74, AAV rh.8, and AAVrh. 10. In some aspects, AAV DNA in the rAAV genomes is from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, AAV rh.74, AAV rh.8, AAVrh.10, and AAV-B1. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

In some embodiments, recombinant AAV genomes of the disclosure comprise one or more AAV ITRs flanking a polynucleotide sequence, for example, one or more an antisense sequences that bind to key exon definition elements in the pre-mRNA, inhibiting the recognition of a specific exon by the spliceosome, leading to exclusion of the target exon of DMD from the mature RNA. Thus, in some embodiments, rAAV genomes of the disclosure comprise one or more AAV ITRs flanking a polynucleotide encoding, for example, one or more DMD antisense sequences. Commercial providers such as Ambion Inc. (Austin, TX), Darmacon Inc. (Lafayette, CO), InvivoGen (San Diego, CA), and Molecular Research Laboratories, LLC (Herndon, VA) generate custom inhibitory RNA molecules. In addition, commercial kits are available to produce custom siRNA molecules, such as SILENCER™ siRNA Construction Kit (Ambion Inc., Austin, TX) or psiRNA System (InvivoGen, San Diego, CA).

In some embodiments, therefore, the U7 snRNA gene comprising the DMD inhibitory nucleic acid is cloned as U7-ACCA into an rAAV vector. Thus, embodiments of the disclosure include an rAAV genome comprising a nucleic acid comprising the nucleotide sequence set out in SEQ ID NO: 1 or a variant thereof comprising a nucleotide sequence having sequence identity to SEQ ID NO: 1 as disclosed herein in the detailed description.

In some embodiments, the viral vector is a pseudotyped AAV, containing ITRs from one AAV serotype and capsid proteins from a different AAV serotype. In some embodiments, the pseudo-typed AAV is AAV2/9 (i.e., an AAV containing AAV2 ITRs and AAV9 capsid proteins). In some embodiments, the pseudotyped AAV is AAV2/8 (i.e., an AAV containing AAV2 ITRs and AAV8 capsid proteins). In some embodiments, the pseudotyped AAV is AAV2/1 (i.e., an AAV containing AAV2 ITRs and AAV1 capsid proteins).

In some embodiments, the AAV contains a recombinant capsid protein, such as a capsid protein containing a chimera of one or more of capsid proteins from AAV1, AAV2, AAV3, AAV4, AAV5, AAV6, AAV7, AAV8, AAV9, AAV-anc80, AAVrh74, AAVrh.8, or AAVrh.10, AAV10, AAV11, AAV12, AAV13, or AAV-B1. Other types of rAAV variants, for example rAAV with capsid mutations, are also contemplated. See, for example, Marsic et al., Molecular Therapy, 22(11): 1900-1909 (2014). As set out herein above, the nucleotide sequences of the genomes of various AAV serotypes are known in the art.

In some embodiments, DNA plasmids are provided which comprise rAAV genomes as described herein. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-B1 and AAV rh74. Production of pseudotyped rAAV is disclosed in, for example, WO 01/83692 which is incorporated by reference herein in its entirety.

In some embodiments, packaging cells are provided. Packaging cells are created in order to have a cell line that stably expresses all the necessary components for AAV particle production. Retroviral vectors are created by removal of the retroviral gag, pol, and env genes. These are replaced by the therapeutic gene. In order to produce vector particles, a packaging cell is essential. Packaging cell lines provide all the viral proteins required for capsid production and the virion maturation of the vector. Thus, packaging cell lines are made so that they contain the gag, pol and env genes. Following insertion of the desired gene into in the retroviral DNA vector, and maintenance of the proper packaging cell line, it is now a simple matter to prepare retroviral vectors

For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

Thus, further provided in some embodiments are packaging cells that produce infectious rAAV. Packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In some embodiments, therefore, a method of generating a packaging cell to create a cell line that stably expresses all the necessary components for AAV particle production is provided. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy et al., 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbiol. and Immunol. 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); Mclaughlin et al., J. Virol., 62:1963 (1988); and Lebkowski et al., 1988 Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658.776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 (1995); Paul et al., Human Gene Therapy, 4:609-615 (1993); Clark et al., Gene Therapy, 3:1124-1132 (1996); U.S. Pat. No. 5,786,211; U.S. Pat. No. 5,871,982; U.S. Pat. No. 6,258,595; and McCarty, Mol. Ther., 16(10): 1648-1656 (2008). The foregoing documents are hereby incorporated by reference in their entirety herein, with particular emphasis on those sections of the documents relating to rAAV production. The production and use of various types of rAAV are specifically contemplated and exemplified. Recombinant AAV (i.e., infectious encapsidated rAAV particles) are thus provided herein. In some aspects, genomes of the rAAV lack AAV rep and cap genes; that is, there is no AAV rep or cap DNA between the ITRs of the genomes of the rAAV. In some embodiments, the AAV is a recombinant linear AAV (rAAV), a single-stranded AAV (ssAAV), or a recombinant self-complementary AAV (scAAV).

The disclosure thus provides packaging cells that produce infectious rAAV. In one embodiment, packaging cells are stably transformed cancer cells, such as Hela cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV, in some aspects, are purified by methods standard in the art, such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In some embodiments, the disclosure provides a composition or compositions comprising a nucleic acid or a vector, e.g., such as a viral vector, as described herein. Thus, compositions comprising delivery vehicles (such as rAAV) described herein are provided. In various aspects, such compositions also comprise a pharmaceutically acceptable carrier. In various aspects, such compositions also comprise other ingredients, such as a diluent, excipients, and/or adjuvant. Acceptable carriers, diluents, excipients, and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁵, about 1×10¹⁶, or more DNase resistant particles (DRP) [or viral genomes (vg)] per ml.

In some embodiments, the dose of rAAV administered is about 1.0×10¹⁰ vg/kg to about 1.0×10¹⁶ vg/kg. In some aspects, 1.0×10¹⁰ vg/kg is also designated 1.0 E10 vg/kg, which is simply an alternative way of indicating the scientific notation. Likewise, 10¹¹ is equivalent to E11, and the like. In some aspects, the dose of rAAV administered is about 1.0×10¹¹ vg/kg to about 1.0×10¹⁵ vg/kg. In some aspects the dose of rAAV is about 1.0×10¹⁰ vg/kg, about 2.0×10¹⁰ vg/kg, about 3.0×10¹⁰ vg/kg, about 4.0×10¹⁰ vg/kg, about 5.0×10¹⁰ vg/kg, about 6.0×10¹⁰ vg/kg, about 7.0×10¹⁰ vg/kg, about 8.0×10¹⁰ vg/kg, about 9.0×10¹⁰ about 1.0×10¹¹ vg/kg, about 2.0×10¹¹ vg/kg, about 3.0×10¹¹ vg/kg, about 4.0×10¹¹ vg/kg, about 5.0×10¹¹ vg/kg, about 6.0×10¹¹ vg/kg, about 7.0×10¹¹ vg/kg, about 8.0×10¹¹ vg/kg, about 9.0×10¹¹ vg/kg, about 1.0×10¹² vg/kg, about 2.0×10¹² vg/kg, about 3.0×10¹² vg/kg, about 4.0×10¹² vg/kg, about 5.0×10¹² vg/kg, about 6.0×10¹² vg/kg, about 7.0×10¹² vg/kg, about 8.0×10¹² vg/kg, about 9.0×10¹² vg/kg, about 1.0×10¹³ vg/kg, about 2.0×10¹³ vg/kg, about 3.0×10¹³ vg/kg, about 4.0×10¹³ vg/kg, about 5.0×10¹³ vg/kg, about 6.0×10¹³ vg/kg, about 7.0×10¹³ vg/kg, about 8.0×10¹³ vg/kg, about 9.0×10¹³ vg/kg, about 1.0×10¹⁴ vg/kg, about 2.0×10¹⁴ vg/kg, about 3.0×10¹⁴ vg/kg, about 4.0×10¹⁴ vg/kg, about 5.0×10¹⁴ vg/kg, about 6.0×10¹⁴ vg/kg, about 7.0×10¹⁴ vg/kg, about 8.0×10¹⁴ vg/kg, about 9.0×10¹⁴ vg/kg, about 1.0×10¹⁵ vg/kg, about 2.0×10¹⁵ vg/kg, about 3.0×10¹⁵ vg/kg, about 4.0×10¹⁵ vg/kg, about 5.0×10¹⁵ vg/kg, about 6.0×10¹⁵ vg/kg, about 7.0×10¹⁵ vg/kg, about 8.0×10¹⁵ vg/kg, about 9.0×10¹⁵ vg/kg, or about 1.0×1016 vg/kg.

In some aspects, the dose is about 1.0×10¹¹ vg/kg to about 1.0×10¹⁵ vg/kg. In some aspects, the dose is about 1.0×10¹³ vg/kg to about 5.0×10¹³ vg/kg. In some aspects, the dose is about 2.0×10¹³ vg/kg to about 4.0×10¹³ vg/kg. In some aspects, the dose is about 3.0×10¹³ vg/kg.

In some aspects, an initial dose is followed by a second greater dose. In some aspects, an initial dose is followed by a second same dose. In some aspects, an initial dose is followed by one or more lesser doses. In some aspects, an initial dose is followed by multiple doses which are the same or greater doses.

In some aspects, the initial dose is about 1.0×10¹³ vg/kg to about 5.0×10¹³ vg/kg. In some aspects, the initial dose is about 2.0×10¹³ vg/kg to about 4.0×10¹³ vg/kg. In some aspects, the initial dose is about 3.0×10¹³ vg/kg. In some aspects, the second dose is about 6.0×10¹³ vg/kg to about 4.0×10¹⁴ vg/kg. In some aspects, the second dose is about 7.0×10¹³ vg/kg to about 9.0×10¹³ vg/kg. In some aspects, the second dose is about 8.0×10¹³ vg/kg.

Methods of transducing a target cell with a delivery vehicle (such as rAAV), in vivo or in vitro, are contemplated. Transduction of cells with an rAAV of the disclosure results in sustained expression of antisense sequence that binds to key exon definition elements in the pre-mRNA of the DMD gene, inhibiting the recognition of a specific exon by the spliceosome, leading to exclusion of the target exon from the mature DMD RNA. The disclosure thus provides rAAV and methods of administering/delivering rAAV which express antisense sequence that binds to key exon definition elements in the pre-mRNA, inhibiting the recognition of a specific exon by the spliceosome, leading to exclusion of the target exon from the mature RNA to a subject. In some aspects, the subject is a mammal. In some aspects, the mammal is a human. These methods include transducing cells and tissues (including, but not limited to, tissues such as muscle) with one or more rAAV described herein. Transduction may be carried out with gene cassettes comprising cell-specific control elements. The term “transduction” is used to refer to, as an example, the administration/delivery of u7snRNA comprising antisense sequence, e.g., U7-ACCA, to a target cell either in vivo or in vitro, via a replication-deficient rAAV described herein resulting in the expression of functional forms of the dystrophin protein by the target cell. Two such known functional forms are wild-type dystrophin and the IRES driven isoform. As described herein, while skipping of a single copy of exon 2 restores wild-type protein, skipping both copies is not detrimental because it activates the IRES and allows for the production of a highly-functional dystrophin protein.

The in vivo methods comprise the step of administering an effective dose, or effective multiple doses, of a composition comprising a delivery vehicle (such as rAAV) to a subject (including a human subject) in need thereof. Thus, methods are provided of administering an effective dose (or doses, administered essentially simultaneously or doses given at intervals) of rAAV described herein to a subject in need thereof. If the dose or doses is administered prior to development of a disorder/disease, the administration is prophylactic. If the dose or doses is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival.

In some embodiments, compositions and methods of the disclosure are used in treating, ameliorating, or preventing a disease, such as a muscular dystrophy (MD). In various aspects, such MD is Duchenne Muscular Dystrophy (DMD). DMD, an X-linked degenerative muscle disorder, is the most common severe childhood form of muscular dystrophy affecting around 1:5200 male births (Mendell et al., Ann Neurol 71, 304-313 (2012)). Symptoms of generalized muscle weakness first appear at ages 3-5 and progress into a loss of ambulation by age 13, with death typically occurring in the third decade of life due to cardiomyopathy or respiratory insufficiency (Passamano et al., Acta Myol 31, 121-125 (2012); Duchenne, The Pathology of Paralysis with Muscular Degeneration (Paralysie Myosclerotique), or Paralysis with Apparent Hypertrophy. Br Med J 2, 541-542 (1867)). DMD is caused by mutations that disrupt the open reading frame in the DMD gene, which encodes dystrophin (Juan-Mateu et al., PLoS One 10, e0135189 (2015)), a large (427 kDa) multifunctional protein that is localized at the subsarcolemmal region of myofibers, where it plays an important role in protecting the sarcolemma from mechanical damage caused by muscle contraction (Petrof et al., Proc Natl Acad Sci USA 90, 3710-3714 (1993)). In other various aspects, such MD is Becker Muscular Dystrophy (BMD). The presence of a partially functional dystrophin protein occurs with mutations that maintain an open reading frame (ORF), resulting in the milder allelic disorder BMD (Wein et al., Nature Medicine 20, 992-1000 (2014); Monaco, Trends Biochem Sci 14, 412-415 (1989)). BMD, like DMD, is a genetic disorder that gradually makes the body's muscles weaker and smaller. BMD affects the muscles of the hips, pelvis, thighs, and shoulders, as well as the heart, but is known to cause less severe problems than DMD. Because of the variety of in-frame mutations resulting in a variety of partially functional proteins, BMD has a broad phenotypic spectrum with, for example, loss of ambulation ranging from the late teenage years to late adulthood.

In families known to carry pathological DMD or BMD mutations, the methods of the disclosure, in various aspects, are methods of preventing disease and they are carried out before the onset of disease. In other various aspects, the methods of the disclosure are carried out after diagnosis and, therefore, are methods of treating or ameliorating disease.

Molecular, biochemical, histological, and functional outcome measures demonstrate the therapeutic efficacy of the methods. Outcome measures are described, for example, in Chapters 32, 35 and 43 of Dyck and Thomas, Peripheral Neuropathy, Elsevier Saunders, Philadelphia, PA, 4^{th} Edition, Volume 1 (2005) and in Burgess et al., Methods Mol. Biol., 602: 347-393 (2010). Outcome measures include, but are not limited to, one or more of the exclusion of the target exon from the mature RNA, reduction or elimination of mutant DMD mRNA or protein in affected tissues, and the expression of a functional form of dystrophin. The expression of functional dystrophin in the cell is detected by measuring the dystrophin protein level by methods known in the art including, but not limited to, Western blot, immunofluorescence, or immunohistochemistry in muscle biopsied before and after administration of the rAAV to determine the improvement.

In some embodiments, the level of functional dystrophin gene expression or protein expression in a cell of the subject is increased after administration of the rAAV as compared to the level of functional dystrophin gene expression or protein expression before administration of the rAAV. In some aspects, expression of a functional form of dystrophin is increased by at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%. In various aspects, improved muscle strength, improved muscle function, and/or improved mobility and stamina show an improvement by at least about 2%, at least about 5%, at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%.

Other outcome measures include measuring the level of serum creatinine kinase (CK) in the subject before and after treatment. Increased CK levels are a hallmark of muscle damage. In Duchenne patients, CK levels are significantly increased above the normal range (10 to 100 times the normal level since birth). When elevated CK levels are found in a blood sample, it usually means muscle is being disintegrated by some abnormal process, such as a muscular dystrophy or inflammation. Thus, a positive therapeutic outcome for treatment with the methods of the disclosure is a reduction in the level of serum creatinine kinase after administration of the rAAV as compared to the level of serum creatinine kinase before administration of the rAAV.

Other outcome measure include measuring to determine if there is improved muscle strength, improved muscle function, improved mobility, improved stamina, or a combination of two or more thereof in the subject after treatment. Such outcome measures are important in determining muscular dystrophy progression in the subject and are measured by various tests known in the art. Some of these tests include, but are not limited to, the six minute walk test, time to rise test, ascend 4 steps test, ascend and descend 4 steps test, North Star Ambulatory Assessment (NSAA) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

Combination therapies are also contemplated by the disclosure. Combination as used herein includes both simultaneous treatment and sequential treatments. Combinations of methods described herein with standard medical treatments and supportive care are specifically contemplated, as are combinations with therapies, such as glucocorticoids. All types of glucocorticoids are included for use in the combination therapies disclosed herein. Such glucocorticoids include, but are not limited to, prednisone, prednisolone, dexamethasone, deflazacort, beclomethasone, betamethasone, budesonide, cortisone, hydrocortisone, methylprednisolone, and triamcinolone.

Administration of an effective dose of a nucleic acid, viral vector, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. In some aspects, an effective dose is delivered by a systemic route of administration, i.e., systemic administration. Systemic administration is a route of administration into the circulatory system so that the entire body is affected. Such systemic administration, in various aspects, takes place via enteral administration (absorption of the drug through the gastrointestinal tract) or parenteral administration (generally via injection, infusion, or implantation). In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the disclosure, in various aspects, are chosen and/or matched by those skilled in the art taking into account the condition or state of the disease or disorder being treated, the condition, state, or age of the subject, and the target cells/tissue(s) that are to express the nucleic acid or protein.

In particular, actual administration of delivery vehicle (such as rAAV) may be accomplished by using any physical method that will transport the delivery vehicle (such as rAAV) into a target cell of an animal. Administration includes, but is not limited to, injection into muscle, the bloodstream and/or directly into the nervous system or liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The disclosure also provides a kit comprising a nucleic acid, vector, or composition of the disclosure or produced according to a process of the disclosure. In the context of the disclosure, the term “kit” means two or more components, one of which corresponds to a nucleic acid, vector, or composition of the disclosure, and the other which corresponds to a container, recipient, instructions, or otherwise. A kit, therefore, in various aspects, is a set of products that are sufficient to achieve a certain goal, which can be marketed as a single unit.

The kit may comprise one or more recipients (such as vials, ampoules, containers, syringes, bottles, bags) of any appropriate shape, size and material containing the nucleic acid, vector, or composition of the disclosure in an appropriate dosage for administration (see above). The kit may additionally contain directions or instructions for use (e.g. in the form of a leaflet or instruction manual), means for administering the nucleic acid, vector, or composition, such as a syringe, pump, infuser or the like, means for reconstituting the nucleic acid, vector, or composition and/or means for diluting the nucleic acid, vector, or composition.

The disclosure also provides kits for a single dose of administration unit or for multiple doses. In some embodiments, the disclosure provides kits containing single-chambered and multi-chambered pre-filled syringes.

It is noted that as used herein, the singular forms “a”, “an”, and “the”, include plural references unless the context clearly indicates otherwise. Thus, for example, reference to “a nucleic acid” includes one or more of such different nucleic acids and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the disclosure described herein. Such equivalents are intended to be encompassed by the disclosure.

The term “and/or” wherever used herein includes the meaning of “and”, “or” and “all or any other combination of the elements connected by said term.”

The term “about” or “approximately” as used herein means within 20%, preferably within 10%, and more preferably within 5% of a given value or range. It includes, however, also the concrete number, e.g., about 10 includes 10.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein, “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this disclosure is not limited to the particular methodology, protocols, material, reagents, and substances, etc., described herein and as such can vary. The terminology used herein is for the purpose of describing particular embodiments only, and is not intended to limit the scope of the subject matter of the disclosure, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material.

A better understanding of the disclosure and of its advantages will be obtained from the following examples, offered for illustrative purposes only. The examples are not intended to limit the scope of the disclosure.

### EXAMPLES

Aspects and embodiments of the disclosure are illustrated by the following examples.

### Example 1

### Materials and Methods

### Constructs

The mouse U7 snRNA gene and smOPT sequences were cloned as U7-ACCA containing two U7snRNA antisense masking the acceptor site and two U7snRNA antisense masking the splice donor site (Wein et al., Nature Medicine 20, 992-1000 (2014); Goyenvalle et al., Science 306, 1796-1799 (2004)). These were cloned in tandem in a self-complementary AAV vector using an Xbal restriction site. All plasmids constructs were sequence verified. Both ITR and self-complementary AAV backbone were checked using respectively Smal and Mscl.

### U7snRNA AAV Vector Design and Production

Vector for this study was produced in the Viral Vector Core at The Research Institute at Nationwide Children's Hospital. Serotype 9 recombinant adeno-associated virus (rAAV9) vectors were produced by a modified cross-packaging approach using an adenovirus-free, triple plasmid DNA transfection (CaPO₄ precipitation) method in human embryonic kidney 293 cells (Rabinowitz et al., J. Virol. 2002; 76: 791-801). Four independent U7snRNA cassettes containing either a copy of an antisense sequence to the splice acceptor (sequence A) or splice donor (sequence C) site were packaged into a scAAV2/9 (scAAV9.U7.ACCA) according to previously published methods (Wein et al., Nat Med 2014; 20: 992-1000). The production plasmids were: (i) scAAV9.U7.ACCA, (ii) rep2-cap9 AAV helper plasmids encoding cap serotype 9, and (iii) an adenovirus type 5 helper plasmid (pAdhelper) expressing adenovirus E2A, E4 ORF6, and VA I/II RNA genes.

scAAV9.U7-ACCA and scAAV1.U7-ACCA also were produced in the Viral Vector Core at The Research Institute at Nationwide Children's Hospital via three plasmid DNA transfection of human HEK 293 VVC Master Cell Bank cells with: (i) the pAAV.U7-ACCA vector plasmid, (ii) an AAV9 or AAV1 helper plasmid containing the AAV rep2 and cap9 or cap1 wild-type genes, and (iii) the helper adenovirus plasmid pHELP. Cells were cultivated in Corning Cell Stacks (Corning) and lysed, using methodology standard to the NCH VVC. Two days post transfection, both cells and media were collected.

Physical titer determination was determined based on degradation of non-encapsidated DNA following digestion of viral capsids. Released encapsidated DNA was quantified by qPCR to determine the DNAase Resistant Particle (DRP) titer. The DRP titer (also referred to as vector genomes [vg]) were determined for scAAV9.U7.ACCA or scAAV1.U7-ACCA products using real-time quantitative PCR (qPCR) using dilutions of a linearized plasmid standard containing U7 amplicon as assay standards. The DRP quantitative PCR (qPCR) assay involves serial dilution of the test sample (10⁻² to 10⁻⁵) and sequential digestion with DNase I and Proteinase K, followed by qPCR analysis. DNA detection was accomplished using sequence specific primers targeting a primer/probe combination specific to the construct sequence (probe: 5′-ACGTAGATAAGTAGCATGGCGGGTTA-3′ (SEQ ID NO: 4); Fw primer: 5′-agctcctatgttgttaTCTAGAG-3′ (SEQ ID NO: 5); Rv primer: 5′-CTAGGGGTTCCTTGTAGTTAATG-3′ (SEQ ID NO: 6)) and amplified using a fluorescently tagged probe hybridizing to the U7 amplicon. Titration of the encapsidated viral genome were performed using quantitative PCR-based titration method runned on a Prism 7500 Taqman detector system (PE Applied Biosystems).

### Study Design

Because DMD almost exclusively affects males, only male mice were used in this study. Mice were housed in a barrier facility with HEPA-filtered air that is AAALAC accredited, and maintained with a 12-hour light/dark cycle. Animals were clustered by groups; no randomization was used. AAV injections were conducted in a nonblinded fashion, but all dissection and most experiments were performed in blinded approaches since animals were clustered by groups. The sample sizes were determined based on previous experience and every legend contains the number of animals used in the study. No statistical methods were used to predetermine sample size prior to experimentation.

### Experimental Animal use and Animal Studies

All experimental procedures involving animals in this study were reviewed and approved by the research institute at Nationwide Children's Hospital's Institutional Animal Care and Use Committee.

Stocks of Dup2 mice were bred and maintained in standardized conditions in the vivarium at Nationwide Children's Research Institute (IACUC protocol #AR10-00002 and IBCSC protocol #IBS00000201). Mice were kept under at 12:12 h dark:light cycle with free access to a low fat diet and water.

For the intramuscular injection studies, eight week old Dup2 mice were injected into the tibialis anterior (TA) with 5 doses of scAAV9.U7.ACCA between 2.0×10¹⁰ vg to 2.0×10¹² total vector genomes (N=10 limbs/dose for molecular studies) and sacrificed 4 weeks later. At sacrifice, TA, gastrocnemius, triceps, diaphragm and heart were removed and frozen in liquid nitrogen-cooled isopentane.

For the intravenous studies, an initial dose escalation study was conducted wherein Dup2 mice were systemically injected via the tail vein at 8 weeks of age with six doses of scAAV9.U7.ACCA between 1.8×10¹² vg/kg and 4.7×10¹⁴ vg/kg (n≥3 mice per dose). Mice were taken down four weeks post-injection and tibialis anterior, gastrocnemius, triceps, diaphragm and heart were removed and frozen in liquid nitrogen cooled isopentane. Increasing treatment time to twelve weeks, a second dose escalation study was conducted following a single systemic injection into the tail vein of 8 week old Dup2 mice with three doses of scAAV9.U7.ACCA between 5.8×10¹³ vg/kg and 4.7×10¹⁴ vg/kg (n≥3 mice per dose).

### Tail Vein Injection

Injection was performed using a tail vein apparatus. The tail was warmed via light bulb to enlarge the veins. Once visible, AAV9 or PBS was injected with 1.9e12 vg (7.6e13 vg/kg) of scAAV9.U7-ACCA in 300 μl total of PBS, or PBS alone, using a 33G gas-tight Hamilton syringe. Following injection, a sterile cotton pad was placed on the injection site and held with pressure until bleeding ceased.

### Facial Vein Injection

One to two-day old pups were anesthetized via placement on ice. Anesthetized pups were placed on a clean surface and held steady between the thumb and index finger, and an insulin needle was inserted into the cranial vein about 1 mm above the eye (either side) and 0.5-1 mm deep. Mice were injected with 1.8e11 vg (1.8e14 vg/kg) of scAAV9.U7-ACCA in 50 μl total of PBS, or PBS alone, using a 33G gas-tight Hamilton syringe. Following the injection, pups were placed on a heating pad to recover.

### Glucocorticoid Treatment

Alpha-methylprednisolone (PDN, M3781, Sigma) was administered by intraperitoneal injection (IP, 12 mg per kg per day) for 3 weeks, 5 times per week. Control animals received phosphate buffered saline (PBS).

### Muscle Preparation

Mice dissection was using standard techniques. Muscles were collected and were either snap frozen or mounted for cryosections, which were cut at 10 μM for immunofluorescence and H&E staining. The tibialis anterior (ta), gastrocnemius (gastroc), quadriceps (quad) and triceps from the right side of all treatment groups were analyzed. Tissues/organs for histopathology studies were collected and fixed in 10% neutral buffered formalin (10% NBF).

### Cell Culture

Primary human cells from skin biopsy were extracted by enzymatic dissociation with collagenase 1A (Sigma-Aldrich). DMEM, 20% fetal bovine serum (FBS), 100 mg/mL penicillin, 100 mg/mL streptomycin, and L-glutamin (Life Technologies™) was used to expand the cells. FibroMyoD cell lines were obtained by infecting cell lines as described in (Wein et al., Hum Mutat 31, 136-142 (2010)).

For transdifferentiation of fibroblasts into myoblasts and myotubes, poly-D-lysine and laminin-coated 10 cm dishes were used (354455, BD Biosciences). Fibroblasts were seeded in a 10 cm coated dish at 50% confluency. When cells reached 70% confluency, media was changed to complete myoblast media ((SkBM-2 complete, Promocell) +4 μg/ml of freshly made doxycycline). Medium was switched to myotube media ((C-23061, Skeletal Muscle Cell Differentiation Medium, Promocell) +4 μg/ml of fresh doxycycline (Wein et al., (2010), supra) when myoblasts reached 95% confluent. Medium was changed every 4 days and cells were collected for protein extraction at day 7-14 post-differentiation, depending on the maturation of myotubes, which varied between cell lines.

### AAV Infection In Vitro

scAAV1.U7-ACCA (3×10¹⁰ for a 6 well plate, or 1.5×10¹¹ vg for a 10 cm plate) was added when myoblast proliferation media was switched to myotube media. Cells were lysed either 3 days post infection for RNA analysis or 7-14 days post infection for protein extraction. RNA extractions were performed using 1 ml of TRIzol (Life Technologies) per well of 6-well plate, according to the manufacturer's instructions.

### Exon Skipping Analysis

Total RNA was isolated from tissue samples and analyzed by reverse transcription (RT) followed by PCR. Total RNA was isolated from frozen muscle sections (15×40 μM sections) using Trizol according to the manufacturers protocol (Life Technologies, 15596018). For each sample, 1 μg of total RNA was used to generate cDNA by RT-PCR using random hexamer primers according to the manufacturer's protocol (Thermo Scientific, Ferk1672) and then used for a single PCR of 35 cycles using 1 μg of RNA and a mixture of random hexamer and oligo(dT) for each RT reaction. mRNA was then amplified via primers specific to the DMD 5′UTR and the exon 3-4 junction as follows. PCR amplification was performed using 2×Master Mix (K0172, ThermoScientific) and 150 ng of RT product as template, using a forward primer-5′UTR Forward: (5′-TACCTAAGCCTCCTGGAGCA-3′ (SEQ ID NO: 2) and a reverse primer to the junction of exon 3 and 4—Exon 3/4 Junction Reverse: (5′-CTTTTGGCAGTTTTTGCCCTGTA-3′ (SEQ ID NO: 3)).

The possible transcripts included a duplicated exon 2 (340 bp), wild type with a single copy of exon 2 (278 bp) and a 42 transcript that had zero copies of exon 2 (216 bp). Following electrophoresis, PCR products were electrophoresed on a 2% agarose gel and imaged using a Gel Logic 200 Imaging System (Kodak). Quantification of Dup2, WT or Del2 transcript was performed using ImageJ (an open source platform for scientific image analysis), and each band plotted in Prism (GraphPad) as a percentage of the overall dystrophin transcript. The images were then used to quantify the relative amount of each transcript using ImageJ software 14. Using the “Gel” function of ImageJ, a box was drawn in each lane around the area where the three possible transcripts would be. A histogram of each sample was then generated and the area under the curve was measured. The sum of the area under the three peaks were added together and then individual peak area was measured as a percentage of the total.

### Protein Extraction and Western Blotting

Cells (experiment performed in duplicate) and mouse muscles lysates were prepared using the same lysis buffer (50 mM Tris-150 mM NaCl, 0.05%NP-40, digitonin (Sigma), pH solution at 7.4; with 1% digitonin, and protease and phosphatases inhibitors (1860932, Thermo inc.)). 150 μl of this lysis buffer was used for 100 mg of tissue. This solution was then put on ice for 30min, and then tissue was lysed using a metal bead (1 min at 30 Hz, TissueLyser II, Qiagen). These last two steps were repeated, and then the lysate was centrifuged at 14000 g for 20min at 4° C. The supernatant was mixed with a classical 4× Laemmli buffer and boiled for 5 min at 100° C. 50-150 μg of protein were run on a precast 3-8% Tris-Acetate gel (NuPage, Life Science) for 16 h at 80V (4° C.). Using a nitrocellulose membrane, gels were transferred overnight at 300 mA using a cooling device. Rabbit polyclonal antibodies against the C-terminal end of dystrophin were used (1:250, PA1-21011, ThermoScientific or 1:400, 15277, Abcam). Utrophin was stained using Mancho3 (1:50, clone 8A4, a gift from Dr Glenn Morris and the MDA Monoclonal Antibody Resource (www.glennmorris.org.uk). Myosin Heavy Chain (MF20 (1:200), from the University of Iowa Developmental Studies Hybridoma Bank (DSHB)) was used for in vitro studies and α-actinin (1:5000, A-7811, Sigma) was used for in vivo studies. Both were used as a loading control. Primary antibodies were incubated for 1 h at RT. Membranes were then washed five times for 5 min with 0.1% Tween in TBS. Secondary antibodies ((anti-mouse IgG (H+L) (IRDye® 680CW Conjugate) and anti-rabbit IgG (H+L) (IRDye@ 800CW Conjugate; both 1:5000 dilution)) were incubated for 1 h RT. All antibodies were diluted in half Odyssey blocking buffer (Licor®) and half Tris buffered saline with Tween (TBST) (Sigma-Aldrich). Following secondary incubation, 5×5 min with 0.1% Tween washes were performed followed by a ddH2O rincing. Both IRDye@ signals were simultaneously scanned using the Odyssey® CLx (LI-COR). The signals for both dystrophin, utrophin and α-actinin were quantified using ImageJ.

With some experiments, 25×40 μm thick sections of cryopreserved tissues were used for western blotting to quantify amount of restored dystrophin protein. Extracted protein was run on 3-8% Tris-Acetate protein gels and visualized using specific primary antibodies against dystrophin or α-actinin the corresponding Licor secondary antibodies IRDye 680 or IRDye 800 (as set out above). Blots were imaged using the Licor Odyssey CLx and quantified using ImageJ.

### Immunofluorescence

Cryopreserved tissues were used for immunofluorescence staining to identify muscle fibers expressing dystrophin protein (DMD). Sections (10 μM) of frozen muscle were permeabilized (phosphate buffered saline (PBS), 2% Normal goat serum (NGS) and 0.1% Triton™-X (Sigma-Aldrich)) and then blocked with 15% NGS in PBS. The primary antibody was a rat monoclonal anti-dystrophin (1:400, Abcam, ab15277) and the secondary antibody (Goat anti-Rabbit, Thermo Fisher A-11011) was conjugated with AlexaFluor 568 (1:250; Invitrogen, A21069). Slides were then mounted using a 2.5% polyvinyl alcohol/1,4 diazabicyclo[2.2.2]octane solution. Sections were imaged under a fluorescent microscope.

### Immunoblot Analysis

Protein extractions were conducted starting with 25 sections (40 μM) and 100 μl of lysis buffer containing a base buffer, a phosphatase inhibitor (PhosStop, Roche, 4906845001) and a protease inhibitor (Halt Protease Inhibitor Cocktail, Fisher, 78430). Steel beads were added to the tissue, which was homogenized using the Tissuelyser II (Qiagen) for 2 min at a rate of 30/sec. Lysates were then incubated on ice and spun down; the supernatant was removed and stored at −80═ C. until immunoblotting, and the cell debris was discarded. For immunoblotting, 50 μg C57BL/6 muscle tissue protein or 150 μg Dup2 muscle tissue protein was loaded with a loading dye (1× laemmli) on a 3-8% Tris Acetate gel (Life Technologies, EA0378BOX). The gels were run for 30 min at 80 V, and then for 4 hours at 120 V. Protein was transferred to a nitrocellulose membrane (Fisher, 09-301-108) overnight at 4° C. in transfer buffer (Invitrogen, NP00061) at 50 V. The membranes were then exposed using a rat monoclonal anti-dystrophin primary antibody (1:200, Abcam, ab15277) and mouse monoclonal α-actinin primary antibody (1:5000, Fisher, MA122863) followed by IRDye α-rabbit 680 and α-mouse 800 (Licor, 926-68071 and 926-32210) secondary antibody. The membrane was scanned on the Odyssey CLx and imaged using Image Studio 14. Single color channel images were using Image Studio and then imported into ImageJ for quantification. For each channel, a box was drawn in each lane around the protein band using the “Gel” function in ImageJ. A histogram of each sample was then generated and the area under the curve was measured. Dystrophin was normalized to α-actinin by dividing the area of the dystrophin band by the area of the α-actinin band for each sample. If the same sample was run on multiple immunoblots, the median was taken to represent that particular sample. The C57BL/6 measurement was determined by taking the mean of all wild type samples. Both treated Dup2 and control Dup2 measurements were reported as a percentage of the wild type (n≥3).

Force Generation and Protection from Eccentric Contractions

Physiologic studies were conducted on TA muscles from 12 week old mice that had been treated with an intramuscular injection of 3.1×10¹¹ vg at 8 weeks (N≥5 limbs). For systemic studies, the mice were 20-21 weeks old following 12 weeks of treatment (N≥12 limbs). The force study procedure was conducted using a modified version of Hakim's procedure (Hakim et al., J Appl. Physiol. (1985) 2011; 110:1656-1663; Wein et al., Nat. Med. 2014; 20:992-1000). Initial anesthetization of mice was conducted by giving an intraperitoneal injection of a cocktail containing five times their weight (i.e., the weight of the mouse) of 25 mg/mL ketamine and twice their weight of 2.5 mg/ml xylazine. The skin fascia and connective tissue were removed from around the tibialis anterior (TA). Throughout the procedure, the TA muscle was constantly moistened with 0.9% saline. A knot was tied to the distal TA tendon with a 4-0 suture and the tendon was cut. The excess suture thread was then knotted again, leaving a loop to attach the tendon to the force transducer. The mouse was then positioned on the platform which was kept at 37° for the duration of the experiment. The leg limb was secured to the platform by putting a pin through the knee cap and taping down the foot. The loop of suture attached to the TA tendon was then attached to a 205B dual-mode servomotor transducer (Aurora Scientific, Aurora, ON, Canada). Finally, two electrical probes were placed in the biceps femoral muscle near the sciatic nerve for stimulation.

The resting force was set to between 3-4 g for a 10 minute equilibrium period. The TA muscle was then stimulated at the optimal length (Lₒ, mm) and active tetanic muscle force was recorded to give the absolute force measurement using the Lab View-based DMC program (Aurora Scientific). The muscle was then run through a 10-step passive stretch protocol during which stimulation was applied to determine the force drop following repeated eccentric contractions. At each step, the TA muscle was passively strained 10% of the Lₒ. Once the protocol was complete, the mice were given a lethal dose of ketamine/xylazine and the TA muscle was removed and weighed. The cross-sectional area measured as mass (g)/(muscle density×ratio of fiber length×Lₒ) where muscle density is 1.06 mg/mm³ and the ratio of fiber length in the TA is 0.6 (Burkholder et al., J Morphol 1994; 221:177-190). The cross-sectional area using the muscle weight and Lₒ were applied to the absolute force measurement to give the specific force measurement. Statistical significance was assessed using a Kruskal-Wallace test assuming nonparametric data in the GraphPad Prism (version 6.03 for Windows, GraphPad Software, San Diego California USA). For all data, the mean and standard deviation were determined for each measurement and subsequently measurements that were more than±1 standard deviation were removed as outliers. The tenth recording for eccentric contractions was used to determine outliers.

### Immunohistochemistry

10 μm thick sections of cryopreserved tissues were air-dried before staining during 30 min. Following rehydration in PBS, sections were permeabilized using permeabilization solution (2% normal goat serum (NGS)+0.1%tritonX-100 in PBS) for 10 min at RT. Then sections were incubated for 1 h with blocking solution (15% NGS in PBS) followed by a 2 hour incubation with an anti-mouse IgG unconjugated fab fragment at RT. Primary antibodies (diluted in PBS+2%NGS) were incubated 2 h or overnight at 4° C. Sections were then washed with washing solution (1× PBS) four times for 5 minutes at RT. For 1 hour at RT, secondary dye-conjugated antibodies diluted in PBS+2%NGS were added to muscle sections. Following 3 washes, DAPI solution was added to stain nucleus. Sections were then mounted using PVA-DABCO. Several primary antibodies were used: dystrophin (1:400; Abcam, ab15277); α1-Laminin (1:400; MAB4656, R&D Systems™); n-Nos1 (1:200; SantaCruz, R-20 (B2613); beta-dystroglycan (1:500, MANDAG2, from DSHB or a gift from Dr Glenn Morris and the MDA Monoclonal Antibody Resource) (http colon-slash-slash www.glennmorris.org.uk); and beta-sarcoglycan (1:50; B-SARC-L-CE; Novocastra). Secondary antibody conjugated with several AlexaFluor dyes (Abcam or Thermofisher) were used at 1:500 dilution. Observations were realized using an Olympus BX61 (Olympus). Acquisitions were taken using a DP controller (Olympus).

### Evans Blue Dye (EBD) and Centronucleation Analysis

To induce muscle injury for Evans blue dye (EBD) analysis, a treadmill exercise program was used. In the first week, four month old mice were trained three times on a treadmill at 10° inclination, running as follows: 0 m/min for 2 min, 5 m/min for 5 min, 10 m/min for 15 min, and 12 m/min for 5-10 min. In the following three weeks (3×/week), mice ran on the treadmill at 2 m/min for 30 min. Before mice euthanasia, the treadmill was used at the same inclination at 5 m/min for 5 min. Following this initial warming up, the speed was increased to +1 m/min until exhaustion. EBD solution (10 mg/ml in PBS) was filtered and injected intraperitoneally (IP) into mice after the last treadmill exercise, and mice were sacrificed 24 hrs later. For analysis, muscle sections were stained with α-laminin to distinguish myofibers, and the percentage of positive Evans blue fibers (labeled in red) were then quantified using ImageJ software. For quantification of centronucleation, H&E staining was performed using standard technique, and fiber counting was performed manually using ImageJ. For both analyses, three sections were processed and counted for each animal. Analysis of the data was performed blindly, but not randomly.

Force Generation and Protection from Eccentric Contractions

Muscle force assessment in the tibialis anterior (TA) muscle was performed using standard techniques (Hakim et al., Methods Mol Biol 709, 75-89 (2011)) modified elsewhere (Wein et al., (2014), supra). In vivo muscle strength was determined by isolating each TA tendon of anesthetized mice. Electrical stimulation was conducted on the muscle to determine force and force drop following repeated eccentric contractions. Specific force was obtained by dividing the maximum tetanic force by the TA muscle cross sectional area. After the eccentric contractions, the mice were then euthanized and the TA muscle was dissected out, weighed and frozen for analyses. Analysis of the data was performed blindly, but not randomly.

### Histology and EBD Quantification

H&E staining was performed using standard protocol. Quantification of both centronucleation and Evans blue dye (EBD) was performed manually using ImageJ. Three sections were processed and counted for each animal. Analysis of the data was performed blindly, but not randomly.

### Statistical Analyses

Statistical analyses were performed using GraphPad Prism (GraphPad). Data normality was tested using D'agostino-Pearson when sample size was sufficient or using the Shapiro-Wilk test. When data had Gaussian distribution, a one-way ANOVA test was used. If not, a Kruskal-Wallis assay was performed. This methodology was applied for all experiments except for the eccentric contraction- induced damage (ECC) where a two-way ANOVA analysis, followed by Bonferroni post-hoc test, were performed.

RNA and Ribosome-Protected mRNA Fragments (RPF) Isolation and Sequencing

Frozen TA muscle from control and scAAV9.U7-ACCA treated Dup2 mice were sectioned on a cryostat and homogenized with a 26-gauge needle in 400 microliters of 20 mM Tris*Cl pH 7.4, 150 mM NaCl, 5 mM MgCl2, 1 mM DTT, 1% Triton X-100, and 100 μg/ml cycloheximide. Ribosome protected fragments were isolated from one half of the homogenate as previously described by Wein et al. (Nature Medicine 20:992-1000, 2014). Total RNA was extracted from the other half of the homogenate using TRIzol and precipitated with isopropanol according to the manufacturer's instruction (Life Technologies). Ribosomal RNA (rRNA) was depleted using Ribo-Zero rRNA Removal Kit Human/Mouse/Rat (Epicentre). TruSeq Stranded Total RNA library kits (Illumina) were used to prepare indexed libraries, and 50 bp reads were generated on an Illumina HiSeq instrument using v4 chemistry (Illumina). Ribosome-protected mRNA fragments (RPF)-Seq libraries were prepared using the TruSeq Small RNA Sample Kit (Illumina) according to the manufacturer's directions, and 50 bp reads were generated on an Illumina HiSeq instrument. Trimmed and filtered RPF-Seq and RNA-Seq reads were mapped to reference genomes using the STAR aligner or to transcript sequences using cross_match. Custom Perl scripts were used to generate read count tables from mapped RPF-Seq and RNA-Seq reads, and edgeR (Bioconductor) was used for model-based read count normalization. The RPF-Seq and RNA-Seq data have been deposited in NCBI's Gene Expression Omnibus and are accessible through GEO Series accession number GSE56148.

### Histopathology Assessment

Following fixation in 10% NBF, all non-skeletal-muscle tissues were embedded in paraffin and sectioned at 12 microns or less, stained with H&E, and microscopically examined for histopathology, by Dr. Christopher Pierson, a physician board-certified in Anatomic Pathology and Neuropathology (Laboratory Medicine/Clinical Pathology, Nationwide Children's Hospital, Columbus, OH). Analysis of the data was performed blindly, but not randomly.

DNA Extraction and qPCR for Biodistribution

All tissues and organs for qPCR/Biodistribution studies were placed into sterile tubes, snap-frozen in liquid nitrogen, and kept at <−70° C. qPCR analyses were performed on samples at 1, 3 and 6 months post-injection. 300 ul DNA lysis buffer (0.05M Tris-HCl pH 8, 0.1M NaCl, 0.1M EDTA, 1% SDS) plus 7.5 ul proteinase K were added to sections and incubated at 55° C. overnight. DNA was quantified and 25 ng of DNA was run in triplicate. The same qPCR primers and probe used for vector titration were used to perform the tissue biodistribution qPCR studies. Data were represented as normalized copy number per 1 ug DNA.

### Example 2

### Systemic Delivery of scAAV9.U7-ACCA Mediates Exon 2 Skipping and Dystrophin Restoration in Muscles of Dup2 Adult Mice

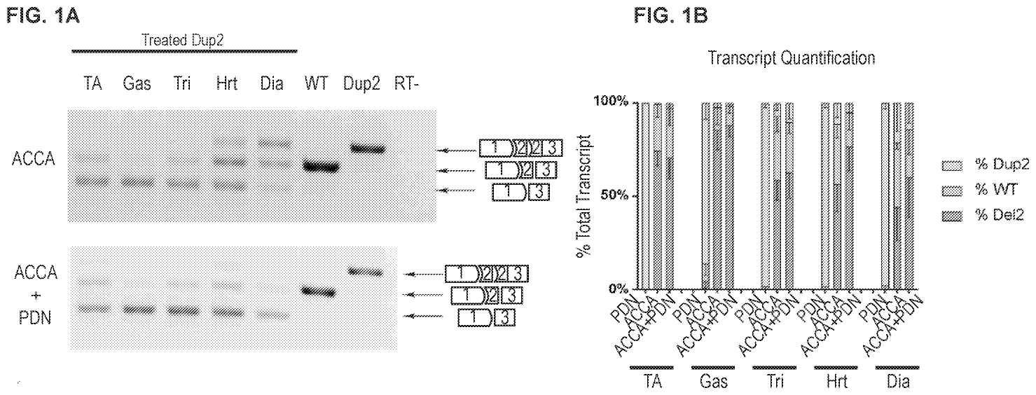

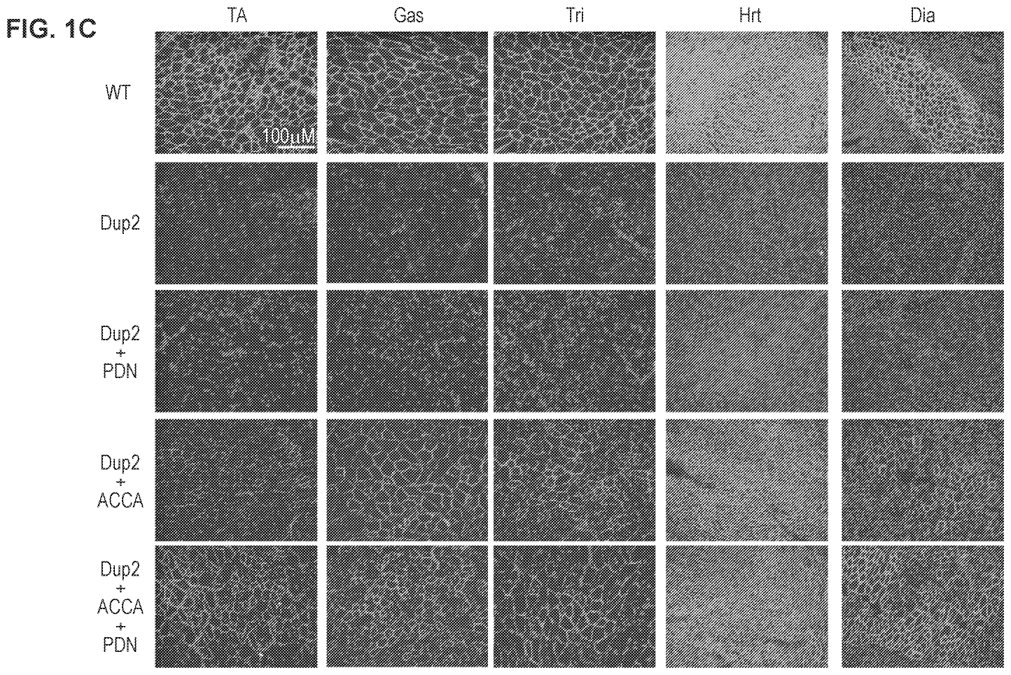

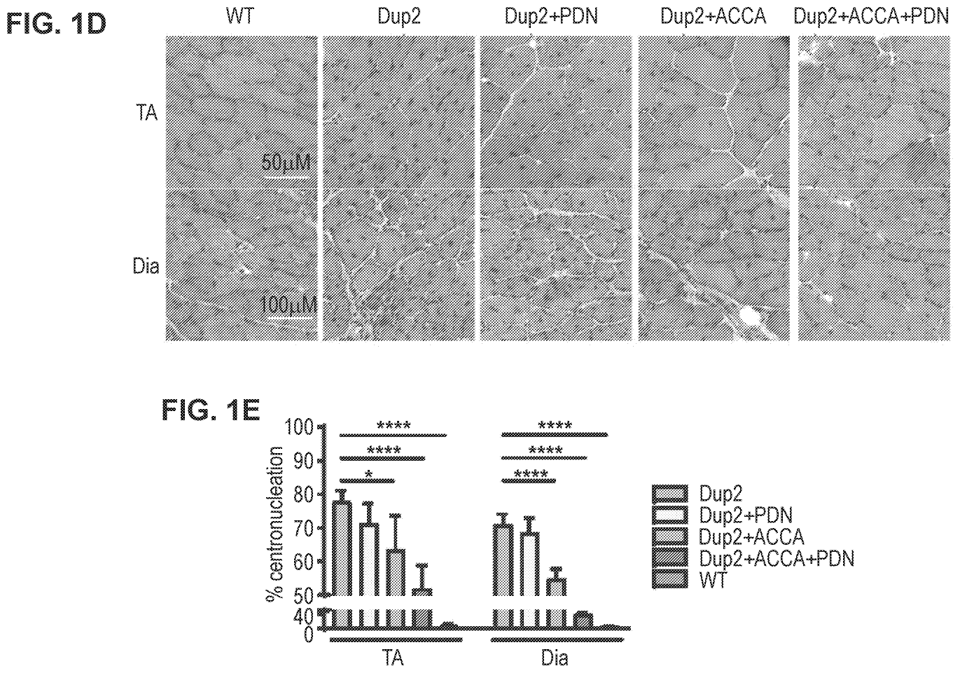

To determine whether systemic delivery of scAAV9.U7-ACCA mediates efficient exon skipping and dystrophin expression, the scAAV9.U7-ACCA vector was injected into 2-month-old Dup2 mice via tail vein injection at 7.6e13vg/kg. Because the activity of the exon 5 IRES is increased in the presence of a glucocorticoid (6α-methylprednisolone 21-hemisuccinate sodium salt (PDN); (Millipore Sigma), two additional groups of mice were also included (PDN alone or ACCA+PDN). Three months later, multiple muscles were collected, including the tibialis anterior (TA), gastrocnemius (Gas), triceps (Tri), heart (Hrt), and diaphragm (Dia). The efficiency of exon skipping was assessed by reverse transcription polymerase chain reaction (RT-PCR). Products were separated on agarose gels and imaged, and quantification of both skipped and unskipped transcripts were performed using ImageJ. Three amplicons were seen in treated animals, representing bands containing two copies (Dup2), one copy (wild-type (WT)), or no copies of exon 2 (Del2) (FIG. 1A). These results demonstrate efficient exon 2 skipping in all muscles (FIG. 1B and Table 1). Both WT and Del2 transcripts are therapeutic, because they result in translation of either the wild type or IRES-driven dystrophin. Overall, more than 80% of the mRNA in both the ACCA and ACCA+PDN groups consisted of therapeutic, skipped transcripts. No significant difference in skipping was found between the ACCA and the ACCA+PDN groups.

TABLE 1

| | | RT-PCR | | | | | | % of EBD |
|---|---|---|---|---|---|---|---|---|
| | | quantification | | | | centronucleation | | positive fibers |
| IM | M | Groups | A1 | A2 | A3 | Groups | B1 | Groups |
| Tail | TA | — | — | — | — | Dup2 | 77.81 ± | Dup2 |
| vein | | | | | | (n = 5) | 3.2 | (n = 4) |
| adult | | PDN | 100 | 0 | 0 | PDN | 71.12 ± | PDN |
| mice | | (n = 4) | | | | (n = 5) | 6 | (n = 4) |
| | | ACCA | 0.4 ± | 25.2 ± | 74.3 ± | ACCA | 63.44 ± | ACCA |
| | | (n = 5) | 1 | 7.1 | 7.9 | (n = 5) | 10.05 | (n = 4) |
| | | PDN + | 2.8 ± | 26.4 ± | 70.7 ± | PDN + | 51.88 ± | PDN + |
| | | ACCA | 2.2 | 9.2 | 11.3 | ACCA | 6.9 | ACCA |
| | | (n = 6) | | | | (n = 5) | | (n = 4) |
| | | — | | | | WT | 10.15 ± | — |
| | | | | | | (n = 2) | 2.9 | |
| | GAS | | | | | — | | Dup2 |
| | | | | | | | | (n = 4) |
| | | PDN | 86 ± | 9.6 ± | 4.3 ± | | | PDN |
| | | (n = 5) | 8.6 | 6.3 | 3.6 | | | (n = 4) |
| | | ACCA | 2.2 ± | 12.2 ± | 85.6 ± | | | ACCA |
| | | (n = 5) | 2.7 | 9.5 | 10.9 | | | (n = 4) |
| | | PDN + | 1.7 ± | 10.1 ± | 88.3 ± | | | PDN + |
| | | ACCA | 2.6 | 3.8 | 6.1 | | | ACCA |
| | | (n = 6) | | | | | | (n = 4) |
| | | — | — | — | — | | | — |
| | TRI | — | — | — | — | | | Dup2 |
| | | | | | | | | (n = 4) |
| | | PDN | 98.3 ± | 1.7 ± | 0 | | | PDN |
| | | (n = 5) | 2.5 | 2.5 | | | | (n = 4) |
| | | ACCA | 6.9 ± | 34.3 ± | 58.8 ± | | | ACCA |
| | | (n = 5) | 4.1 | 8.4 | 10.8 | | | (n = 4) |
| | | PDN + | 10 ± | 26.9 ± | 62.9 ± | | | PDN + |
| | | ACCA | 8.5 | 6 | 13.8 | | | ACCA |
| | | (n = 6) | | | | | | (n = 4) |
| | HEART | | | | | — | | |
| | | PDN | 98.3 ± | 1.6 ± | 0 | | | |
| | | (n = 5) | 2.3 | 2.3 | | | | |
| | | ACCA | 11.1 ± | 32.3 ± | 56.6 ± | | | |
| | | (n = 5) | 7.8 | 7.5 | 14.9 | | | |
| | | PDN + | 5.1 ± | 18.2 ± | 76.6 ± | | | |
| | | ACCA | 5 | 9.3 | 12.9 | | | |
| | | (n = 6) | | | | | | |
| | DIA | — | — | — | — | Dup2 | 70.89 ± | Dup2 |
| | | | | | | | 3.1 | (n = 4) |
| | | PDN | 97.7 ± | 2.3 ± | 0 | PDN | 68.55 ± | PDN |
| | | (n = 5) | 3.1 | 3.1 | | (n = 5) | 4.2 | (n = 4) |
| | | ACCA | 21.1 ± | 34.6 ± | 44.3 ± | ACCA | 54.75 ± | ACCA |
| | | (n = 5) | 15.2 | 3.6 | 17.9 | (n = 5) | 3.1 | (n = 4) |
| | | PDN + | 14.1 ± | 25.6 ± | 60.3 ± | PDN + | 39.95 ± | PDN + |
| | | ACCA | 0.8 | 13.4 | 21.6 | ACCA | 1.5 | ACCA |
| | | (n = 6) | | | | (n = 6) | | (n = 4) |
| | | — | — | — | — | WT | 6.27 ± | — |
| | | | | | | | 0.7 | |

| | | | viral vector | | | |
|---|---|---|---|---|---|---|
| % of EBD | Western blot | | genome | | Western blot | |
| positive fibers | quantification | | quantification | | quantification | |
| C1 | Groups | D1 | Groups | E1 | Groups | F1 |
| 10.34 ± | Dup2 | 4.7 ± | Dup2 | 66 ± | | |
| 18.8 | (n = 1) | 0 | (n = 5) | 123 | | |
| 8.14 ± | PDN | 4.83 ± | PDN | 39 ± | PDN | 0.3 ± |
| 16.2 | (n = 6) | 2.4 | (n = 5) | 37 | (n = 6) | 0.1 |
| 0.32 ± | ACCA | 95.5 ± | ACCA | 32697 ± | ACCA | 0.4 ± |
| 0.36 | (n = 7) | 31.5 | (n = 5) | 49354 | (n = 7) | 0.2 |
| 0.06 ± | PDN + | 127.1 ± | PDN + | 41866 ± | PDN + | 0.47 ± |
| 0.08 | ACCA | 28.3 | ACCA | 23596 | ACCA | 0.2 |
| | (n = 6) | | (n = 5) | | (n = 7) | |
| — | WT | 100 ± | | | | |
| | (n = 4) | 36 | | | | |
| 3.90 ± | Dup2 | 0.5 ± | Dup2 | 15 ± | | |
| 5.4 | (n = 1) | 0 | (n = 5) | 8 | | |
| 3.04 ± | PDN | 2.1 ± | PDN | 15 ± | PDN | 0.2 ± |
| 2.7 | (n = 6) | 1.5 | (n = 6) | 7 | (n = 6) | 0.1 |
| 0.43 ± | ACCA | 41.1 ± | ACCA | 91090 ± | ACCA | 0.4 ± |
| 0.37 | (n = 6) | 22.8 | (n = 5) | 50537 | (n = 7) | 0.4 |
| 0.12 ± | PDN + | 47 ± | PDN + | 171383 ± | PDN + | 0.6 ± |
| 0.18 | ACCA | 17.2 | ACCA | 67140 | ACCA | 0.2 |
| | (n = 7) | | (n = 5) | | (n = 7) | |
| — | WT | 100 ± | | | | |
| | (n = 2) | 20.5 | | | | |
| 14.39 ± | Dup2 | 4.1 ± | Dup2 | 392 ± | | |
| 22.5 | (n = 1) | 0 | (n = 5) | 834 | | |
| 2.57 ± | PDN | 4.8 ± | PDN | 50 ± | PDN | 0.4 ± |
| 1.8 | (n = 6) | 2.4 | (n = 6) | 37 | (n = 6) | 0.1 |
| 0.05 ± | ACCA | 48.1 ± | ACCA | 49476 ± | ACCA | 0.7 ± |
| 0.1 | (n = 5) | 15.6 | (n = 5) | 23824 | (n = 7) | 0.5 |
| 0.29 ± | PDN + | 50.9 ± | PDN + | 62983 ± | PDN + | 0.4 ± |
| 0.5 | ACCA | 10.5 | ACCA | 27586 | ACCA | 0.1 |
| | (n = 7) | | (n = 5) | | (n = 7) | |
| | WT | 100 ± | | | | |
| | (n = 3) | 37 | | | | |
| — | Dup2 | 14.1 ± | Dup2 | 16 ± | | |
| | (n = 1) | 0 | (n = 5) | 7 | | |
| | PDN | 13.7 ± | PDN | 188 ± | PDN | 0.5 ± |
| | (n = 5) | 4 | (n = 6) | 287 | (n = 5) | 0.3 |
| | ACCA | 90.4 ± | ACCA | 54712 ± | ACCA | 0.3 ± |
| | (n = 6) | 34 | (n = 5) | 25460 | (n = 7) | 0.2 |
| | PDN + | 125 ± | PDN + | 89033 ± | PDN + | 0.3 ± |
| | ACCA | 12.1 | ACCA | 37191 | ACCA | 0.2 |
| | (n = 5) | | (n = 5) | | (n = 7) | |
| | WT | 100 ± | | | | |
| | (n = 1) | 0 | | | | |
| 4.57 ± | Dup2 | 5.3 ± | Dup2 | 32 ± | | |
| 7.1 | (n = 1) | 0 | (n = 5) | 30 | | |
| 12.31 ± | PDN | 9.2 ± | PDN | 17 ± | PDN | 0.6 ± |
| 9.1 | (n = 5) | 5.8 | (n = 5) | 17 | (n = 5) | 0.2 |
| 1.45 ± | ACCA | 68.2 ± | ACCA | 880270 ± | ACCA | 0.6 ± |
| 1.5 | (n = 6) | 31.9 | (n = 5) | 1240741 | (n = 7) | 0.3 |
| 0.49 ± | PDN + | 102.9 ± | PDN+ | 53280 ± | PDN+ | 0.7 ± |
| 0.47 | ACCA | 27.3 | ACCA | 12051 | ACCA | 0.1 |
| | (n = 6) | | (n = 5) | | (n = 7) | |
| — | WT | 100 ± | | | | |
| | (n = 1) | 0 | | | | |

IM = Injection Methods
M = Muscles
A1 = % of Dup2 transcript
A2 = % of WT transcript
A3 = % of Del2 transcript
B1 = % of centronucleation
C1 = % of positive fibers
D1 = % of dystrophin
E1 = vector/ug DNA
F1 = ratio of utrophin/a-actinin

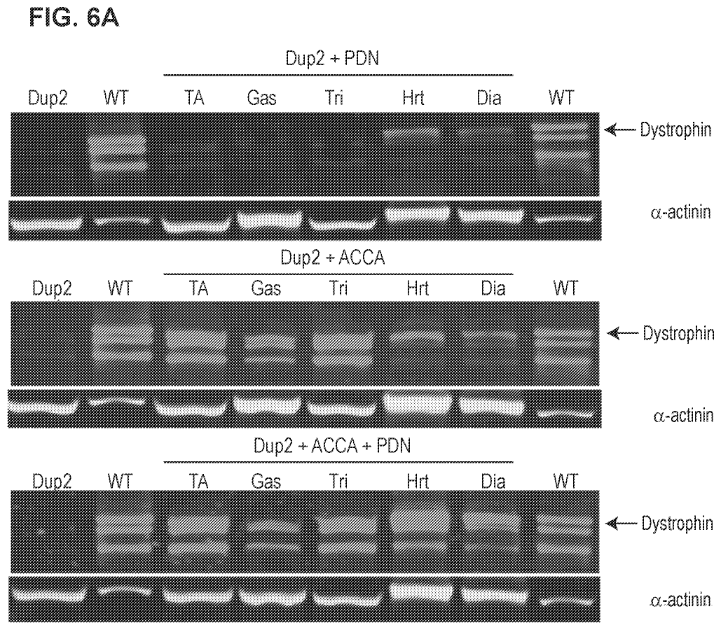

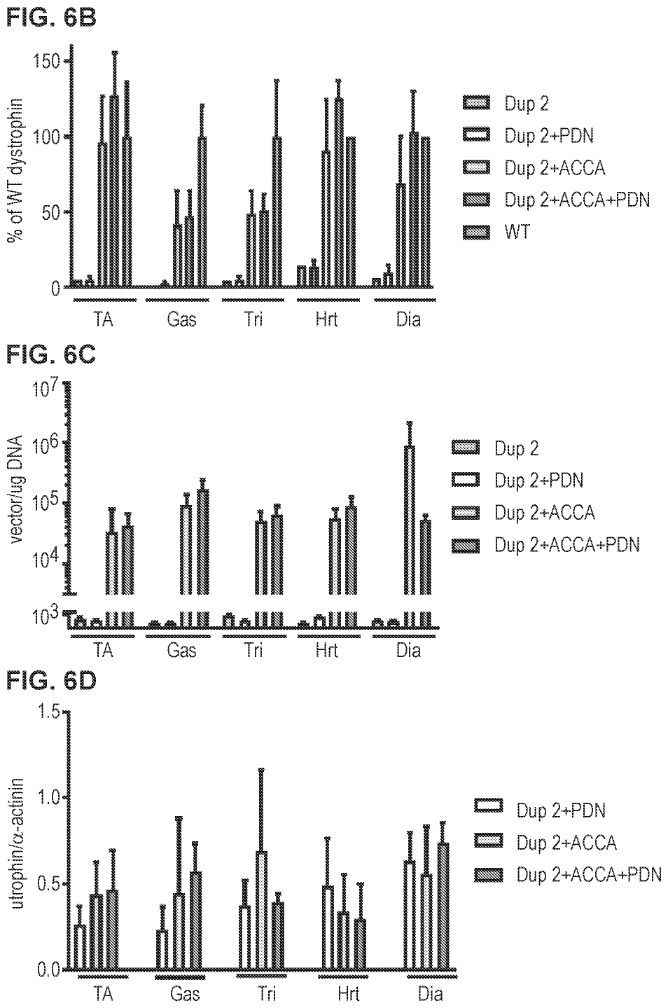

Immunofluorescence analysis using a C-terminal antibody that recognizes both IRES-driven and full-length dystrophin (Fig.1C) showed that treatment, with or without PD, resulted in widespread expression with correct localization to the sarcolemmal membrane. Western blotting quantification was performed in comparison to muscle from wild type (WT) C57BI6 mice (FIG. 6A). The two treated groups (ACCA or ACCA+PDN) showed an overall strong restoration of protein expression, with levels similar to those in the WT mouse in the Hrt, Dia, and TA (FIG. 6B). Variation in expression among muscles, and between the two treatment groups, is not correlated to vector biodistribution, as assessed by qPCR of viral genomes (FIG. 6C). Levels of the dystrophin paralog utrophin, expression of which modifies features of dystrophinopathy in animal models (Deconinck et al., Cell 90, 717-727 (1997)), was unaltered by vector treatment, with or without PDN (FIG. 6D; values summarized in Table 1).

Histopathologic analyses of hematoxylin and eosin (H&E) stained sections of TA and Dia muscles were performed in the two ACCA-treated groups, as well as in PDN-treated Dup2, untreated Dup2, and WT mice (FIG. 1D). ACCA-treated muscles showed decreased signs of myofiber necrosis, and decreased central nucleation compared to controls with best results seen in Dia, where combined treatment resulted in less than 40% centrally nucleated fibers as compared to 70% in the untreated Dup2 mice.

### Example 3

### Rescue of the Dystrophin-Glycoprotein Complex and Muscle Function after Systemic Delivery of scAAV9.U7-ACCA in Dup2 Adult Mice

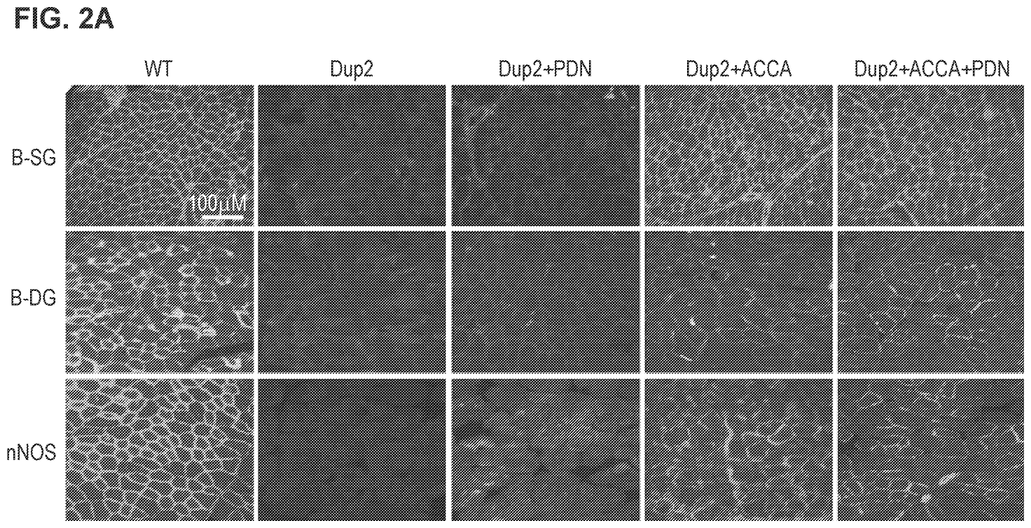

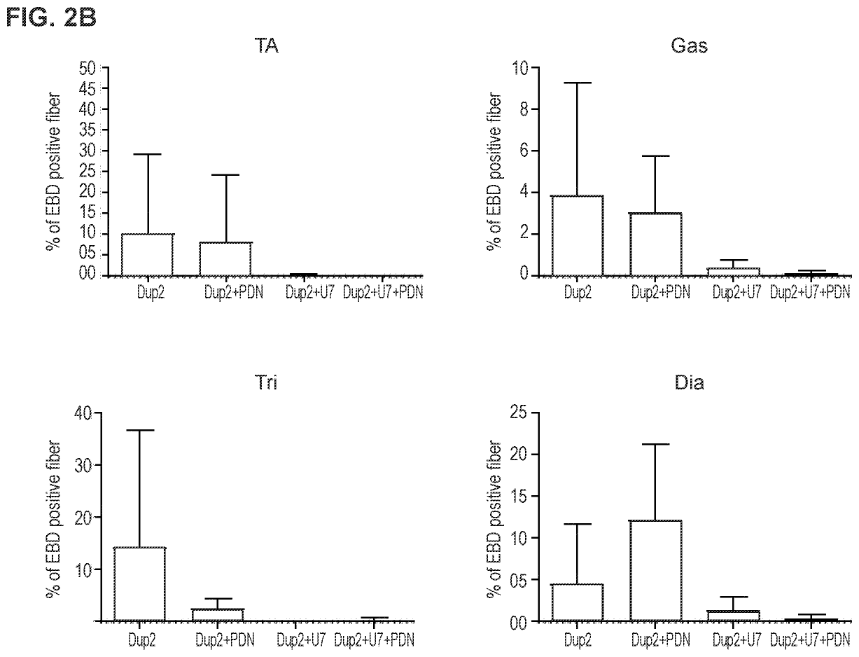

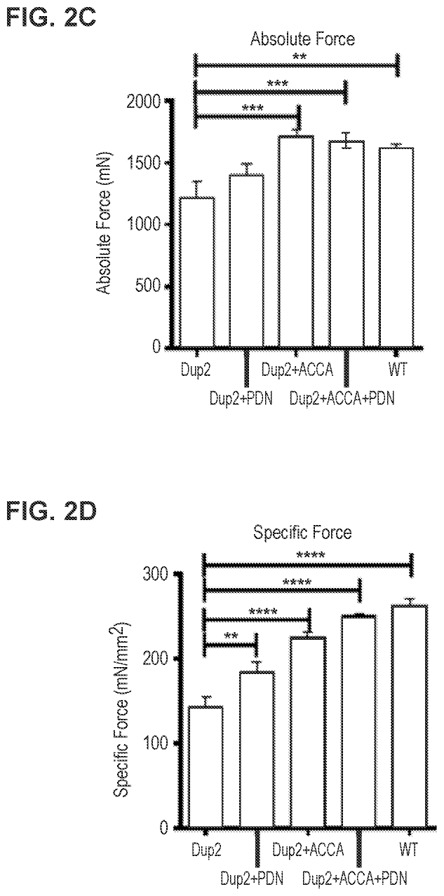

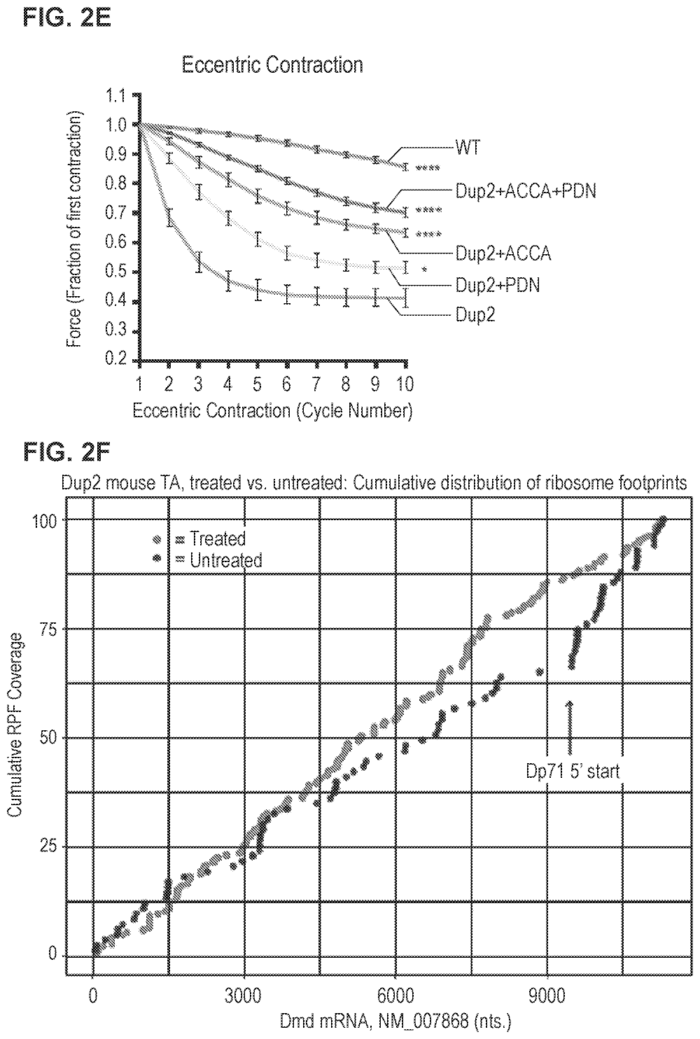

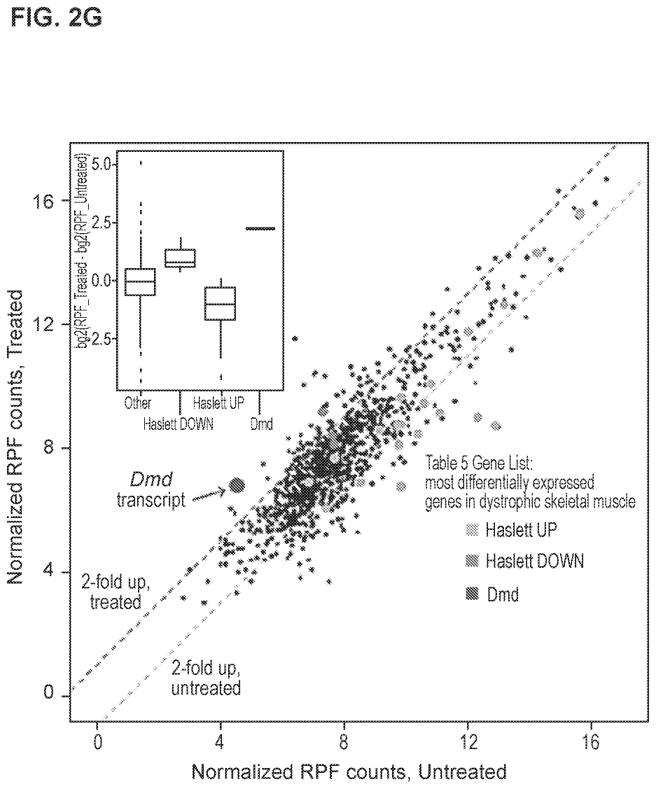

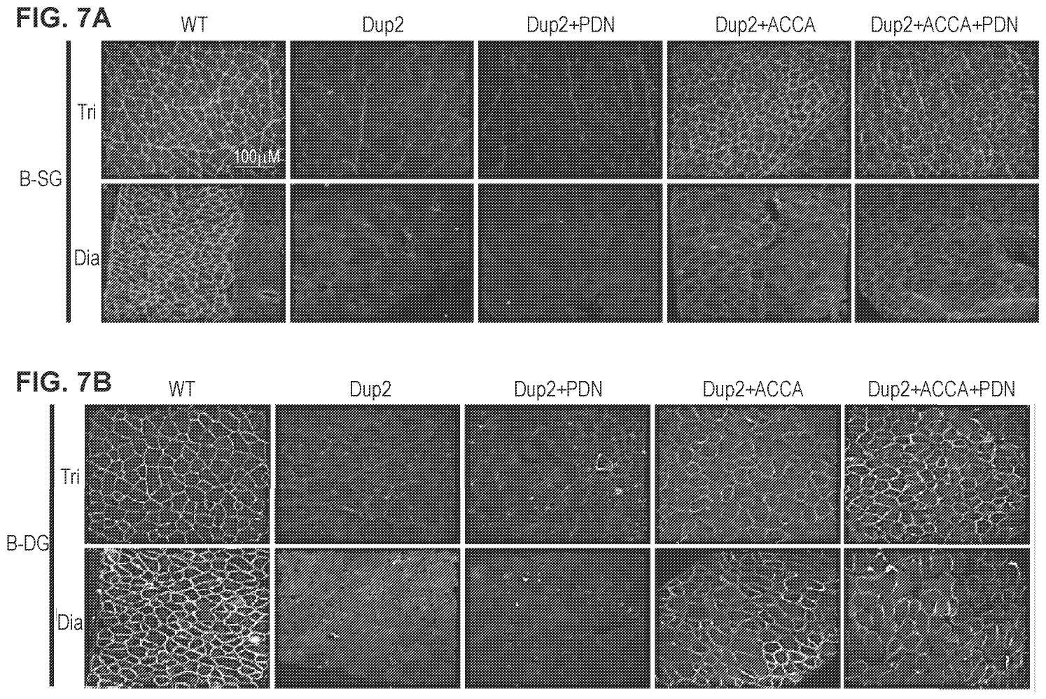

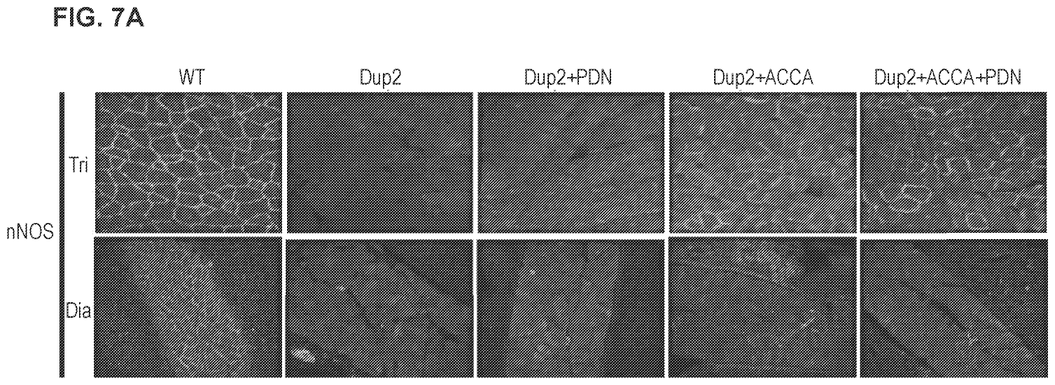

One of the primary roles of dystrophin is to link the cytoskeleton to the extracellular matrix via the dystrophin-glycoprotein complex (DGC). If dystrophin is missing or non-functional, this complex is completely disrupted or mislocalized, and restoration of the complex would be expected from therapeutic dystrophin re-expression. Restoration of the DGC proteins, α-sarcoglycan (α-SG) and β-dystroglycan (β-DG), and of neuronal nitric oxide synthase (nNOS) was carried out. nNOS is a protein that binds to dystrophin to localize to the subsarcolemma region, where it plays a role in signal transduction and has been implicated in DMD pathogenesis (Chang et al., Proc Natl Acad Sci USA 93, 9142-9147 (1996)). Treatment with ACCA or ACCA+PDN resulted in complete recovery of α-SG in skeletal muscle, with less complete restoration in the diaphragm (FIGS. 2A and 7A). The relocalization of β-DG and nNOS after treatment was less evenly distributed, but restoration was observed compared to the non-treated mice, with the exception of the diaphragm where nNOS was not completely restored.

To assess restoration of sarcolemmal integrity, myofiber uptake of Evans Blue Dye (EBM), injected after exhaustive treadmill exercise to provoke muscle fiber injury, was quantified. Overall, a reduction of the uptake of EBD in treated mice was observed (FIG. 2B, Table 1). The reduction did not reach statistical significance, which may be attributable to the high variability in non-U7 treated groups. However, in vivo physiology studies of the TA muscle showed that treatment, with or without PDN, resulted in increases in both absolute and specific forces, and resulted in improvement in force retention following repeated eccentric contractions (FIG. 2E, Table 2). Together, these results demonstrate that scAAV9.U7-ACCA mediated exon skipping, with or without PDN, leads to production of a highly functional dystrophin following systemic delivery in adult Dup2 mice.

TABLE 2

| | Absolute | | Specific | | Eccentric | | | |
|---|---|---|---|---|---|---|---|---|
| | force | | force | | contraction | | | |
| IM | GROUPS | A1 | GROUPS | A1 | GROUPS | B1 | IM | M |
| Tail | Dup2 | 1219 ± | Dup2 | 144 ± | Dup2 | 0.41 ± | facial | TA |
| vein | (n = 5) | 284 | (n = 5) | 25 | (n = 5) | 0.032 | vein | |
| adult | PDN | 1401 ± | PDN | 184 ± | PDN | 0.52 ± | young | |
| mice | (n = 5) | 198 | (n = 6) | 26 | (n = 5) | 0.019 | mice | |
| | ACCA | 1713 ± | ACCA | 225 ± | ACCA | 0.63 ± | | |
| | (n = 10) | 175 | (n = 10) | 21 | (n = 10) | 0.014 | | |
| | PDN + | 1678 ± | PDN + | 251 ± | PDN + | 0.71 ± | | |
| | ACCA | 150 | ACCA | 7 | ACCA | 0.016 | | |
| | (n = 7) | | (n = 8) | | (n = 6) | | | |
| | WT | 1608 ± | WT | 264 ± | WT | 0.86 ± | | |
| | (n = 7) | 117 | (n = 7) | 20 | (n = 5) | 0.012 | | |

| Absolute | | | Specific | | | Eccentric | | |
|---|---|---|---|---|---|---|---|---|
| force | | | force | | | contraction | | |
| GROUPS | C1 | C2 | GROUPS | C1 | C2 | GROUPS | D1 | D1 |
| Dup2 | 927 ± | 1023 ± | Dup2 | 121 ± | 115 ± | Dup2 | 0.57 ± | 0.4 ± |
| (n = 7/9) | 190 | 65 | (n = 7/8) | 24 | 13 | (n = 7/7) | 0.028 | 0.029 |
| ACCA | 854 ± | 696 ± | ACCA | 207 ± | 172 ± | ACCA | 0.75 ± | 0.78 ± |
| (n = 8/6) | 95 | 55 | (n = 7/8) | 13 | 21 | (n = 7/7) | 0.016 | 0.033 |
| WT | 1349 ± | 1494 ± | WT | 236 ± | 225 ± | WT | 0.85 ± | 0.82 ± |
| (n = 10/9) | 137 | 72 | (n = 9/11) | 44 | 19 | (n = 6/13) | 0.007 | 0.019 |

IM = Injection Method
M = Muscle
A1 = Force (mN)
B1 = % force decrease
C1 = Force (mN) 3 Mo post injection
C2 = Force (mN) 6 Mo post injection
D1 = % force decreased

### Example 4

### Correction of Gene Expression Abnormalities after Systemic Delivery of scAAV9.U7-ACCA in Dup2 Adult Mice

RNA from TA specimens was analyzed by ribosome protected fragment analysis (RPF) (FIG. 2F), which allows inferences regarding the translational activity of the mRNA by mapping the distribution of RPFs onto the DMD transcript. It was found that the cumulative distribution of DMD RPF reads covered most of the DMD transcript in the untreated sample. Although the source of this coverage is unknown, it is consistent with a model that would have the source near the 5′ end of the mRNA. Interestingly, there is an inflection point of coverage near the beginning of the Dp71 isoform. In the untreated samples, more than 30% of the total RPF coverage is generated from the Dp71 region. In contrast, the treated sample demonstrated less than 15% of the total RPF coverage mapped to the Dp71 region and had a more uniform coverage across the length of the transcript, consistent with an increased translation from the 5′ end of the mRNA.

In order to determine whether the treatment corrects the transcriptome of the Dup2 muscle, the expression of a group of genes that are dysregulated in DMD patients was evaluated (see Haslett et al., Proc Natl Acad Sci USA 99, 15000-15005 (2002)). RNA-Seq data (Table 3) indicated that treatment restored a ‘non-dystrophic’ gene expression profile by reversing the direction and magnitude of differentially expressed genes previously identified in dystrophic skeletal muscle from DMD patients. Genome-wide normalized RPF-Seq data also showed that untreated Dup2 TA samples have a dystrophic transcriptional profile, whereas treatment resulted in a general correction in the level of expression. Levels of previously downregulated genes were increased and levels of upregulated genes were decreased (FIG. 2G), consistent with a general correction in the global expression pattern. In addition, the DMD transcript showed clearly increased RPF reads in the treated samples, indicating that the treatment specifically increased DMD translation.

TABLE 3
Comparison of fold change direction in AAV9.U7-ACCA treated vs. untreated
mouse skeletal muscle versus human dystrophic vs. normal skeletal muscle

| | | | | | | | log2 Fold | |
|---|---|---|---|---|---|---|---|---|
| | | | | | | | Change | |
| | | | | | | | (Haslett | Reversal of |
| | | | | | | log2 Fold | Table S5, | dystrophic |
| | | | | | | Change | dystrophic | fold-change |
| | | | | | | (AAV9.U7- | vs. normal | by |
| | | | | | | ACCA | human | AAV9.U7- |
| Mouse | | Human | | | | treated vs. | skeletal | ACCA |
| Gene | Ensembl_ID | Gene | GeneChip | Accession | Description | untreated) | muscle)* | treatment |
| Myl1 | ENSMUSG00000061816.15 | MYL1 | 38251_at | AI127424 | myosin, light | −0.62 | 2.91 | YES |
| | | | | | polypeptide 1, | | | |
| | | | | | alkali; | | | |
| | | | | | skeletal, fast | | | |
| Col3a1 | ENSMUSG00000026043.18 | COL3A1 | 32488_at | X14422 | collagen, type III, | −3.45 | 6.30 | YES |
| | | | | | alpha 1 | | | |
| Col1a2 | ENSMUSG00000029661.16 | COL1A2 | 32307_s_at | V00503 | collagen, type I, | −3.40 | 5.61 | YES |
| | | | | | alpha 2 | | | |
| Hspg2 | ENSMUSG00000028763.18 | HSPG2 | 32845_at | M85289 | heparin sulfate | −1.49 | 2.05 | YES |
| | | | | | proteoglycan | | | |
| | | | | | (perlecan) | | | |
| Sparc | ENSMUSG00000018593.13 | SPARC | 671_at | J03040 | secreted protein, | −2.66 | 2.64 | YES |
| | | | | | acidic, | | | |
| | | | | | cysteine-rich | | | |
| | | | | | (osteonectin) | | | |
| Col6a3 | ENSMUSG00000048126.16 | COL6A3 | 38077_at | X52222 | collagen, type VI, | −2.20 | 2.38 | YES |
| | | | | | alpha 3 | | | |
| Scn1b | ENSMUSG00000019194.15 | SCN1B | 38516_at | L10338 | sodium channel, | 0.22 | −2.06 | YES |
| | | | | | voltage-gated, | | | |
| | | | | | type I, beta | | | |
| | | | | | polypeptide | | | |
| Actc1 | ENSMUSG00000068614.7 | ACTC | 39063_at | J00073 | alpha-cardiac actin | −2.61 | 10.63 | YES |
| Sparcl1 | ENSMUSG00000029309.7 | SPARC | 671_at | J03040 | secreted protein, | −0.67 | 2.64 | YES |
| | | | | | acidic, | | | |
| | | | | | cysteine-rich | | | |
| | | | | | (osteonectin) | | | |
| Tuba4a | ENSMUSG00000026202.13 | TUBA4A | 40567_at | X01703 | Tubulin, alpha, | 0.78 | 5.31 | Partial |
| | | | | | brain-specific | | | |
| Pde4d | ENSMUSG00000021699.17 | PDE4D | 38526_at | U02882 | phosphodiesterase | 0.38 | −4.77 | YES |
| | | | | | 4D, cAMP-specific | | | |
| Rpl31 | ENSMUSG00000002500.15 | RPL3L | 38226_at | U65581 | ribosomal protein | 0.35 | −2.22 | YES |
| | | | | | L3-like | | | |
| Ptpn3 | ENSMUSG00000038764.14 | PTPN3 | 1458_at | M64572 | protein tyrosine | −1.08 | −2.29 | Partial |
| | | | | | phosphatase, non- | | | |
| | | | | | receptor type 3 | | | |
| Mn1 | ENSMUSG00000070576.4 | MN1 | 37283_at | X82229 | meningioma 1 | 0.82 | −2.29 | YES |
| Art3 | ENSMUSG00000034842.16 | ART3 | 35134_at | U47054 | ADP- | 0.32 | −4.77 | YES |
| | | | | | ribosyltransferase | | | |
| | | | | | 3 | | | |
| Col5a2 | ENSMUSG00000026042.16 | COL5A2 | 38422_at | Y14690 | collagen, type V, | −2.87 | 3.32 | YES |
| | | | | | alpha 2 | | | |
| Vim | ENSMUSG00000026728.9 | VIM | 34091_s_at | Z19554 | vimentin | −1.48 | 2.41 | YES |
| Col6a1 | ENSMUSG00000001119.7 | COL6A1 | 38722_at | X15880 | collagen, type VI, | −2.26 | 2.31 | YES |
| | | | | | alpha 1 | | | |
| Eef1a1 | ENSMUSG00000037742.14 | EEF1A1 | 1288_s_at | J04617 | eukaryotic translation | −0.94 | 2.05 | YES |
| | | | | | elongation factor 1 | | | |
| | | | | | alpha 1 | | | |
| Reep1 | ENSMUSG00000052852.8 | REEP1 | 36096_at | AL080222 | unknown clone | 0.30 | 2.07 | Partial |
| Lum | ENSMUSG00000036446.5 | LUM | 38038_at | U21128 | lumican | −2.06 | 6.10 | YES |
| Bgn | ENSMUSG00000031375.17 | BGN | 38126_at | J04599 | biglycan | −2.09 | 4.22 | YES |
| Pxdn | ENSMUSG00000020674.17 | PXDN | 39327_at | D86983 | KIAA0230 | −1.82 | 2.25 | YES |
| Myh8 | ENSMUSG00000055775.16 | MYH8 | 34471_at | M36769 | myosin, heavy | −1.92 | 17.82 | YES |
| | | | | | polypeptide 8, | | | |
| | | | | | skeletal muscle, | | | |
| | | | | | perinatal | | | |
| Casq2 | ENSMUSG00000027861.13 | CASQ2 | 36505_at | D55655 | calsequestrin 2 | −0.70 | 2.75 | YES |
| Ube2d1 | ENSMUSG00000019927.6 | UBE2D1 | 1164_at | | Ubiquitin-Conjugating | 0.25 | −3.39 | YES |
| | | | | | Enzyme Ubch5 | | | |
| Rrbp1 | ENSMUSG00000027422.15 | RRBP1 | 33213_g_at | AF006751 | ribosome binding | −0.80 | 1.88 | YES |
| | | | | | protein 1 | | | |
| Clic4 | ENSMUSG00000037242.8 | CLIC4 | 33891_at | AL080061 | chloride intracellular | −0.69 | 2.82 | YES |
| | | | | | channel 4 | | | |
| Aldh2 | ENSMUSG00000029455.14 | ALDH2 | 32747_at | X05409 | aldehyde | 0.25 | −2.60 | YES |
| | | | | | dehydrogenase | | | |
| | | | | | 2 family | | | |
| Ucp3 | ENSMUSG00000032942.14 | UCP3 | 34422_r_at | U82818 | uncoupling protein 3 | −0.94 | −3.30 | Partial |
| Myh3 | ENSMUSG00000020908.14 | MYH3 | 32729_at | X13988 | myosin, heavy | −2.65 | 54.90 | YES |
| | | | | | polypeptide 3, | | | |
| | | | | | skeletal muscle, | | | |
| | | | | | embryonic | | | |
| Pfkfb1 | ENSMUSG00000025271.13 | PFKFB1 | 33980_at | X52638 | 6-phosphofructo-2- | 0.12 | −3.06 | YES |
| | | | | | kinase/fructose-2,6- | | | |
| | | | | | biphosphatase 1 | | | |
| Tubb4b | ENSMUSG00000036752.4 | TUBB4B | 296_at | | tubulin, beta | 0.56 | 3.55 | Partial |
| Gpd11 | ENSMUSG00000050627.13 | GPD1L | 38394_at | D42247 | unknown clone | −0.04 | −2.29 | Partial |
| Arhgef2 | ENSMUSG00000028059.16 | ARHGEF2 | 40099_at | AB014551 | rho/rac guanine | −0.69 | 1.94 | YES |
| | | | | | nucleotide exchange | | | |
| | | | | | factor 2 | | | |
| Mxra8 | ENSMUSG00000029070.9 | MXRA8 | 35219_at | AL050222 | unknown clone | −0.58 | 1.90 | YES |
| Igfbp4 | ENSMUSG00000017493.12 | IGFBP4 | 39781_at | U22982 | insulin-like growth | −1.22 | 2.62 | YES |
| | | | | | factor-binding | | | |
| | | | | | protein 4 | | | |
| Stat6 | ENSMUSG00000002147.18 | STAT6 | 41222_at | AB023228 | MLL septin-like | −0.23 | 2.27 | YES |
| | | | | | fusion | | | |
| Taz | ENSMUSG00000009995.17 | TAZ | 33876_at | AL050107 | transcriptional co- | 0.08 | 3.07 | Partial |
| | | | | | activator with PDZ- | | | |
| | | | | | binding motif | | | |
| Tgfb3 | ENSMUSG00000021253.7 | TGFB3 | 1767_s_at | X14885 | transforming growth | −0.10 | 3.05 | YES |
| | | | | | factor-beta 3 | | | |
| Meg3 | ENSMUSG00000021268.17 | MEG3 | 39026_r_at | AF052114 | maternally | −1.86 | 4.40 | YES |
| | | | | | expressed 3 | | | |
| Maged1 | ENSMUSG00000025151.16 | MAGED1 | 41139_at | W26633 | melanoma antigen, | −0.82 | 2.65 | YES |
| | | | | | family D, 1 | | | |
| Myof | ENSMUSG00000048612.16 | MYOF | 34678_at | AL096713 | myoferlin | −1.57 | 2.91 | YES |
| Pcolce | ENSMUSG00000029718.14 | PCOLCE | 31609_s_at | L33799 | procollagen C- | −2.17 | 2.70 | YES |
| | | | | | endopeptidase | | | |
| | | | | | enhancer | | | |
| Atp1b3 | ENSMUSG00000032412.8 | ATP1B3 | 32563_at | U51478 | ATPase, Na+/K+ | −0.39 | 4.47 | YES |
| | | | | | transporting, beta 3 | | | |
| | | | | | polypeptide | | | |
| Maged2 | ENSMUSG00000025268.15 | MAGED2 | 34859_at | Z98046 | melanoma antigen, | −2.46 | 3.14 | YES |
| | | | | | family D, 2 | | | |
| Actn1 | ENSMUSG00000015143.15 | ACTN1 | 39329_at | X15804 | actinin, alpha 1 | −0.80 | 2.07 | YES |
| Ctsk | ENSMUSG00000028111.4 | CTSK | 38466_at | X82153 | cathepsin K | −3.24 | 3.33 | YES |
| | | | | | (pycnodysostosis) | | | |
| Anxa1 | ENSMUSG00000024659.15 | ANXA1 | 37403_at | X05908 | annexin A1 | −0.97 | 2.08 | YES |
| Runx1 | ENSMUSG00000022952.17 | RUNX1 | 39421_at | D43969 | runt-related | −0.44 | 6.36 | YES |
| | | | | | transcription | | | |
| | | | | | factor 1 | | | |
| Olfml2a | ENSMUSG00000046618.7 | OLFML2A | 38312_at | AL050002 | unknown clone | −1.05 | 2.25 | YES |
| S100a10 | ENSMUSG00000041959.14 | S100A10 | 39338_at | AI221310 | S100 calcium-binding | −1.00 | 2.67 | YES |
| | | | | | protein A10 | | | |
| Iffo1 | ENSMUSG00000038271.17 | IFFO1 | 36030_at | AL080214 | unknown clone | −0.32 | 2.29 | YES |
| Maob | ENSMUSG00000040147.14 | MAOB | 37628_at | M69177 | monoamine oxidase B | 1.03 | −3.35 | YES |
| Lipa | ENSMUSG00000024781.16 | LIPA | 38745_at | X76488 | lipase A, lysosomal | −1.32 | 2.53 | YES |
| | | | | | acid, cholesterol | | | |
| | | | | | esterase | | | |
| Arpc1b | ENSMUSG00000029622.16 | ARPC1B | 39043_at | AF006084 | actin related | −1.08 | 3.19 | YES |
| | | | | | protein 2/3 | | | |
| | | | | | complex, subunit 1B | | | |
| Shc1 | ENSMUSG00000042626.13 | SHC1 | 38118_at | U73377 | SHC (Src homology 2 | −0.65 | 2.19 | YES |
| | | | | | domain-containing) | | | |
| | | | | | transforming protein 1 | | | |
| Fyn | ENSMUSG00000019843.14 | FYN | 40480_s_at | M14333 | FYN oncogene related | −0.82 | 2.12 | YES |
| | | | | | to SRC, FGR, YES | | | |
| Arhgef25 | ENSMUSG00000019467.15 | ARHGEF2 | 40099_at | AB014551 | rho/rac guanine | −0.57 | 1.94 | YES |
| | | | | | nucleotide exchange | | | |
| | | | | | factor 2 | | | |
| Ckap4 | ENSMUSG00000046841.5 | CKAP4 | 32529_at | X69910 | cytoskeleton- | −1.63 | 2.20 | YES |
| | | | | | associated | | | |
| | | | | | protein 4 | | | |
| Gusb | ENSMUSG00000025534.17 | GUSB | 33308_at | M15182 | glucuronidase, | −1.13 | 1.81 | YES |
| | | | | | beta | | | |
| Gpnmb | ENSMUSG00000029816.10 | GPNMB | 38379_at | X76534 | transmembrane | −1.92 | 2.43 | YES |
| | | | | | glycoprotein nmb | | | |
| Syngr1 | ENSMUSG00000022415.12 | SYNGR1 | 31722_at | AL022326 | synaptogyrin 1 | −0.08 | 2.90 | YES |
| S100a4 | ENSMUSG00000001020.8 | S100A4 | 38087_s_at | W72186 | S100 calcium-binding | −2.19 | 2.74 | YES |
| | | | | | protein A4 | | | |
| Washc1 | ENSMUSG00000024101.15 | SHC1 | 38118_at | U73377 | SHC (Src homology 2 | −0.44 | 2.19 | YES |
| | | | | | domain-containing) | | | |
| | | | | | transforming protein 1 | | | |
| Fads3 | ENSMUSG00000024664.5 | FADS3 | 34224_at | AC004770 | fatty acid | −0.54 | 1.83 | YES |
| | | | | | desaturase 3 | | | |
| Lgals3 | ENSMUSG00000050335.17 | LGALS3 | 35367_at | AB006780 | lectin, galactoside- | −2.15 | 2.88 | YES |
| | | | | | binding, soluble, 3 | | | |
| | | | | | (galectin 3) | | | |
| Gpc3 | ENSMUSG00000055653.13 | GPC3 | 39350_at | U50410 | glypican 3 | −0.54 | 2.92 | YES |
| Prcp | ENSMUSG00000061119.7 | PRCP | 36672_at | L13977 | prolylcarboxypeptidase | −1.44 | 2.57 | YES |
| | | | | | (angiotensinase C) | | | |
| Lmod1 | ENSMUSG00000048096.7 | LMOD1 | 37765_at | X54162 | leiomodin 1 | 0.51 | −3.23 | YES |
| Laptm5 | ENSMUSG00000028581.17 | LAPTM5 | 37759_at | U51240 | lysosomal-associated | −2.25 | 3.05 | YES |
| | | | | | multitransmembrane | | | |
| | | | | | protein-5 | | | |
| Sgsh | ENSMUSG00000005043.13 | SGSH | 35626_at | U30894 | N-sulfoglucosamine | −0.90 | 2.31 | YES |
| | | | | | sulfohydrolase | | | |
| | | | | | (sulfamidase) | | | |
| Gamt | ENSMUSG00000020150.13 | GAMT | 37602_at | Z49878 | guanidinoacetate N- | −1.79 | −2.05 | Partial |
| | | | | | methyltransferase | | | |
| Dbn1 | ENSMUSG00000034675.17 | DBN1 | 37981_at | D17530 | drebrin 1 | −1.76 | 1.75 | YES |
| Epb4113 | ENSMUSG00000024044.19 | EPB41L3 | 41385_at | AB023224 | erythrocyte membrane | −0.55 | 4.01 | YES |
| | | | | | protein band 4.1- | | | |
| | | | | | like 3 | | | |
| Actg1 | ENSMUSG00000062825.15 | ACTG1 | 34160_at | X04098 | actin, gamma 1 | −0.14 | 2.04 | YES |

*human data from: Haslett et al., Gene expression comparison of biopsies from Duchenne muscular dystrophy (DMD) and normal skeletal muscle. PNAS Nov. 12, 2002 99 (23) 15000-15005; https://doi.org/10.1073/pnas.192571199

### Example 5

### Early Systemic Delivery of scAAV9.U7-ACCA in Neonatal Dup2 Mice Allows Persistent Exon Skipping and Dystrophin Expression

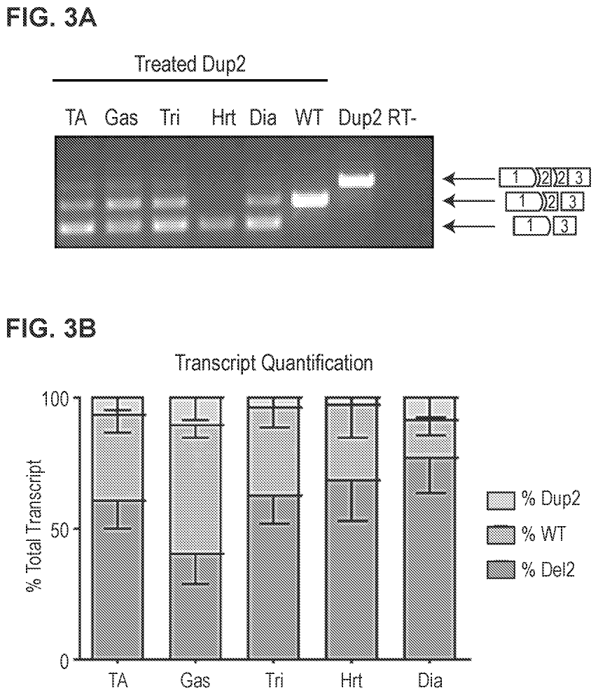

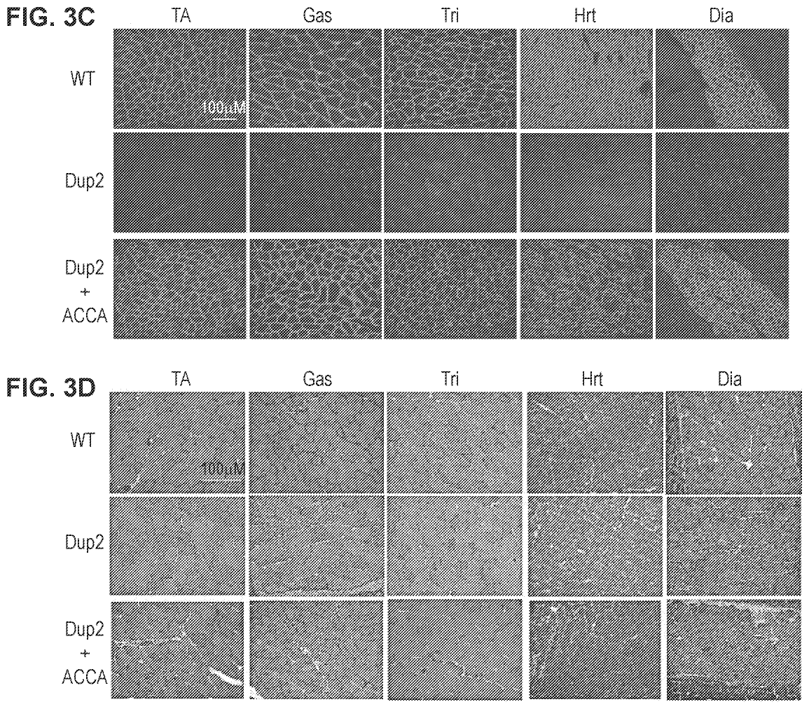

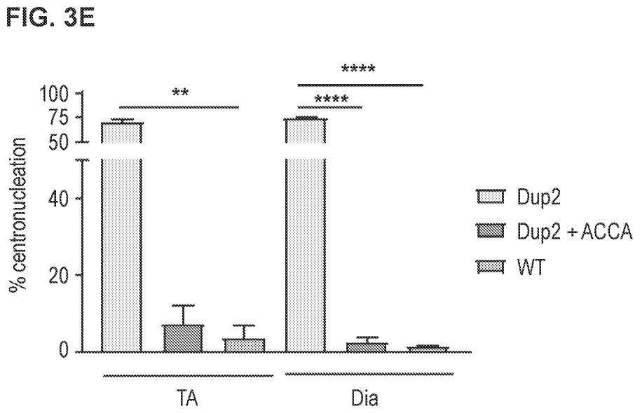

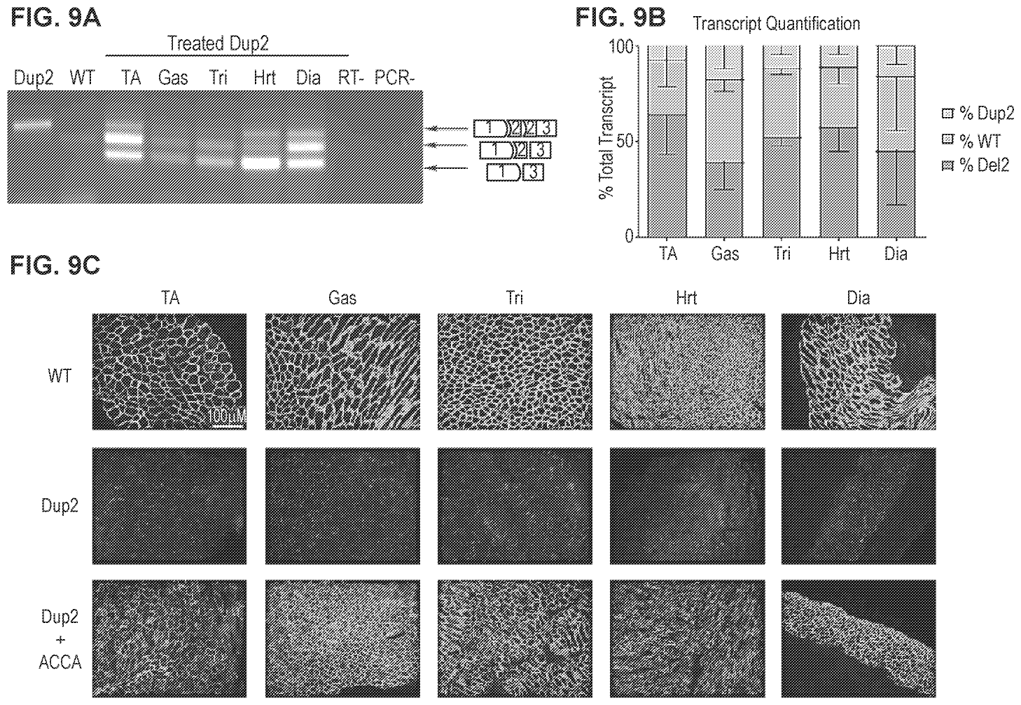

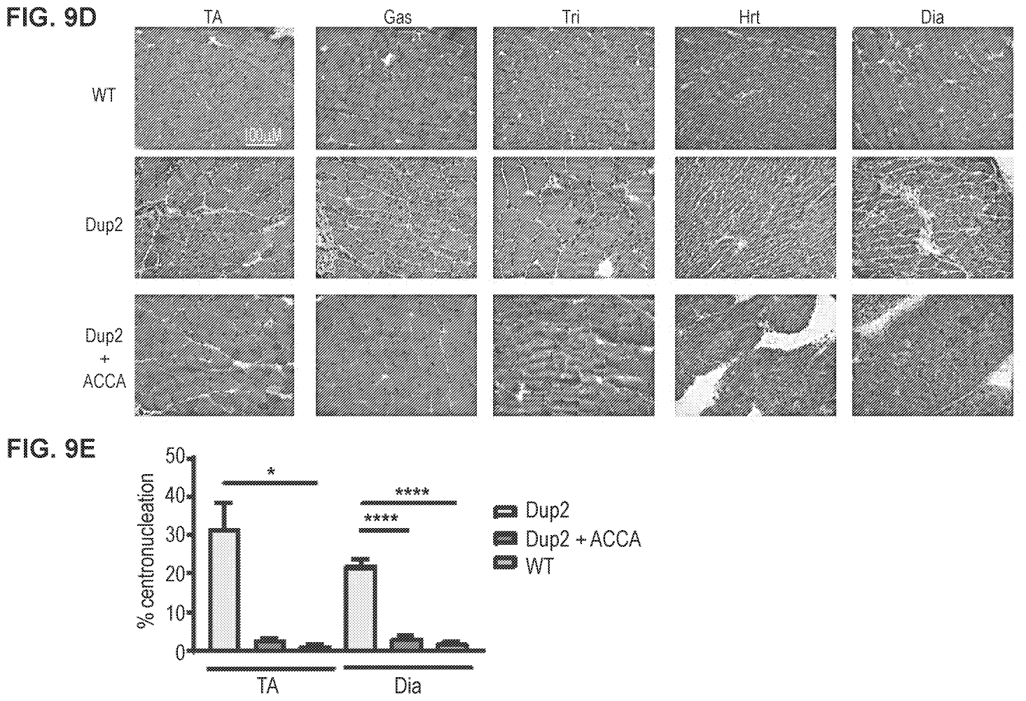

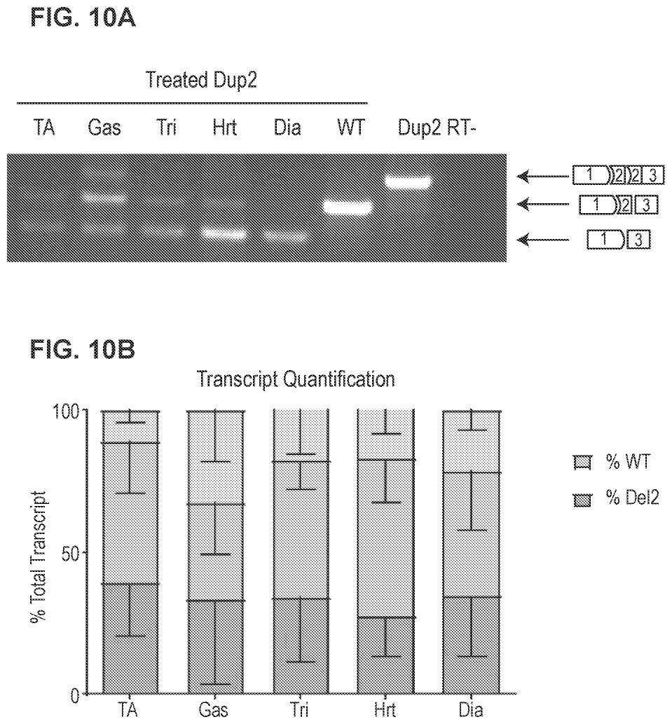

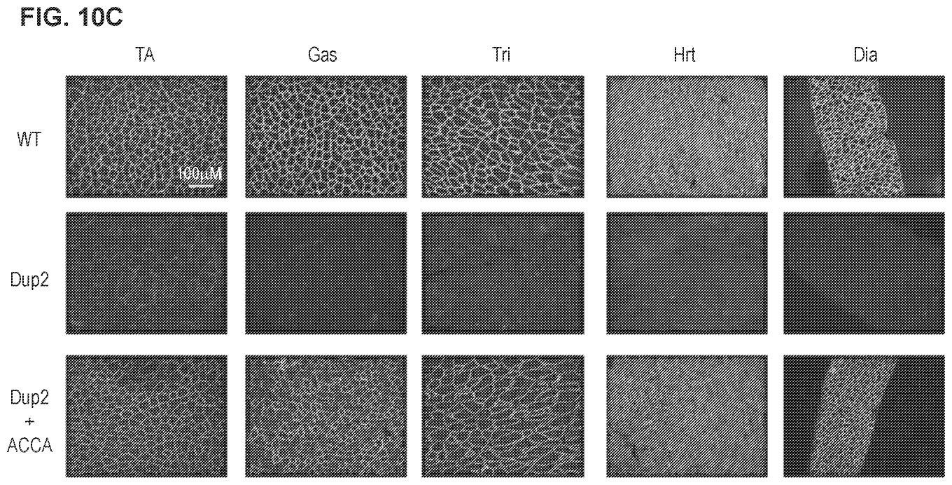

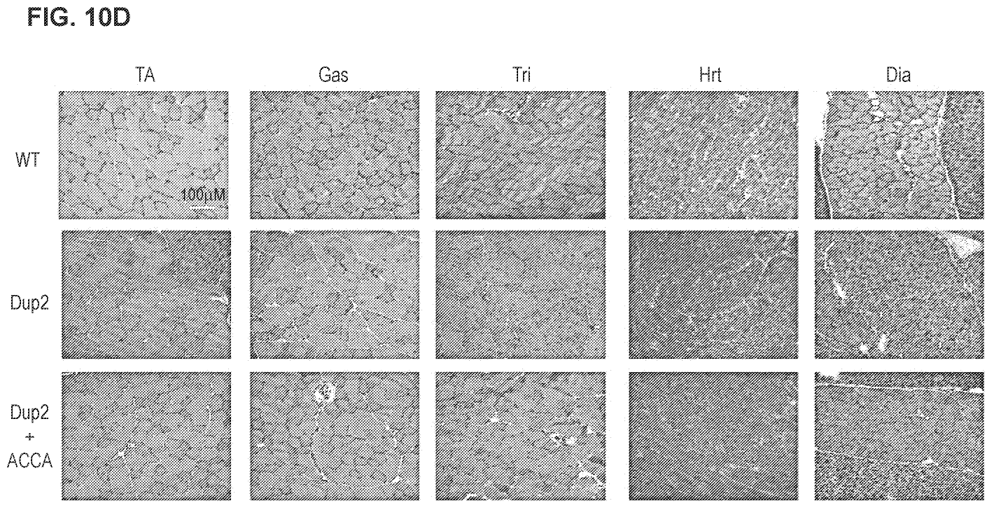

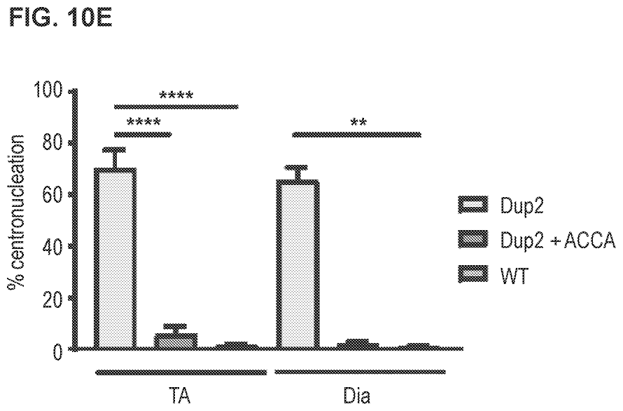

To determine whether treatment earlier in development could prevent muscle degeneration, animals were treated with a single systemic infusion of scAAV9.U7-ACCA at P0-P1 days of age using a dose of 1.8e14 vg/kg. Mice were sacrificed at 1, 3, and 6 months post-injection (FIG. 3A) and DMD exon 2 skipping was quantified by RT-PCR, demonstrating highly efficient exon skipping in multiple tissues (FIG. 3B and Table 4). At 3 months of age, there was a slight decrease in skipping efficiency, ranging between 67% and 89% (FIG. 10); however, this slight decrease was not observed at 6 months of age, where more than 90% of transcripts were WT and Δ2 (Table 4). No differences in viral vector copy number were observed (FIG. 9G), suggesting that the therapeutic vector is stable in tissue until at least 6 months post-injection.

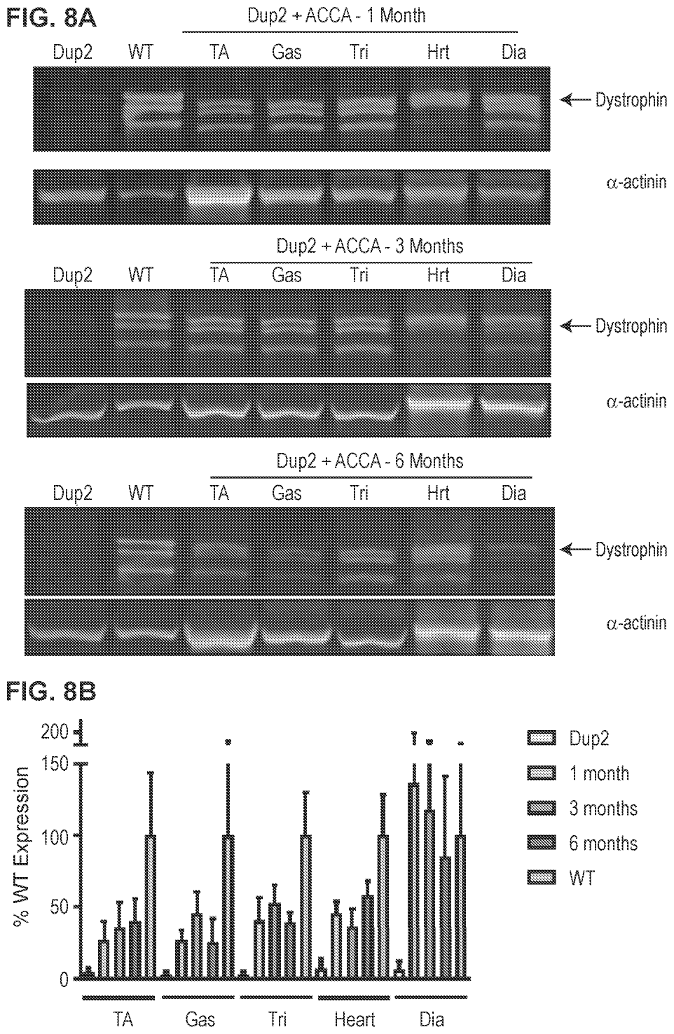

Immunofluorescent analysis showed homogenous and high levels of correctly localized dystrophin in all muscles at 1, 3, and 6 months post-injection (FIGS. 3C, 9C, and 10C, and Table 4). Western blots confirmed persistent and high expression of dystrophin protein, ranging between 25%-60% of dystrophin in all muscles, except for the diaphragm, in which dystrophin protein expression was completely restored after 1 month (FIGS. 8A-B).

TABLE 4

| | | RT-PCR quantification | | | | Western blot | | % of | | viral vector genome | |
|---|---|---|---|---|---|---|---|---|---|---|---|
| | | | % of | % of | % of | quantification | | centronucleation | | quantification | |
| | | | Del2 | WT | Dup2 | | % of | | % of | | % of |
| Injection | | AGE | tran- | tran- | tran- | | WT | | positive | | WT |
| Methods | MUSCLES | GROUPS | script | script | script | GROUPS | dystrophin | GROUPS | fibers | GROUPS | dystrophin |
| facial | TA | | | | | Dup 2 | 4.1 ± | Dup2 | 70.5 ± | ± | |
| vein | | | | | | (n = 4) | 2.8 | (n = 3) | 1.4 | | |
| young | | | | | | WT | 100 ± | WT | 3.5 ± | | |
| mice | | | | | | (n = 3) | 43.6 | (n = 4) | 3.2 | | |
| | | 1 month | 64 ± | 28.7 ± | 7.3 ± | 1 month | 26.9 ± | | | | |
| | | (n = 5) | 20.9 | 14.2 | 7.5 | (n = 5) | 13.2 | | | | |
| | | 3 months | 39.1 ± | 49.8 ± | 11.2 ± | 3 months | 35.7 ± | | | 3 months | 200232 ± |
| | | (n = 7) | 18.7 | 18.3 | 4.2 | (n = 7) | 17.9 | | | (n = 5) | 275171 |
| | | 6 months | 60.4 ± | 33.5 ± | 6.1 ± | 6 months | 40 ± | ACCA | 7.2 ± | 6 months | 84838 ± |
| | | (n = 9) | 10.2 | 7.7 | 5.1 | (n = 9) | 15.8 | (n = 9) | 4.7 | (n = 5) | 65398 |
| | GAS | | | | | Dup 2 | 2.4 ± | | | | |
| | | | | | | (n = 5) | 2.5 | | | | |
| | | | | | | WT | 100 ± | | | | |
| | | | | | | (n = 3) | 62 | | | | |
| | | 1 month | 39.4 ± | 43.2 ± | 17.4 ± | 1 month | 26.8 ± | | | | |
| | | (n = 5) | 14.2 | 6.3 | 11.4 | (n = 5) | 7.2 | | | | |
| | | 3 months | 33 ± | 34.5 ± | 32.6 ± | 3 months | 45.9 ± | | | 3 months | 19369 ± |
| | | (n = 7) | 29.6 | 18.1 | 17.7 | (n = 7) | 14.7 | | | (n = 5) | 11350 |
| | | 6 months | 40.5 ± | 48.9 ± | 10.7 ± | 6 months | 25.2 ± | | | 6 months | 123807 ± |
| | | (n = 9) | 11.5 | 4.8 | 8.5 | (n = 8) | 16.7 | | | (n = 5) | 246935 |
| | TRI | | | | | Dup 2 | 2.6 ± | | | | |
| | | | | | | (n = 4) | 1.8 | | | | |
| | | | | | | WT | 100 ± | | | | |
| | | | | | | (n = 2) | 30.3 | | | | |
| | | 1 month | 52.3 ± | 36.2 ± | 11.5 ± | 1 month | 40.4 ± | | | | |
| | | (n = 4) | 4.4 | 3.1 | 4.7 | (n = 5) | 15.9 | | | | |
| | | 3 months | 34 ± | 48.3 ± | 17.7 ± | 3 months | 52.9 ± | | | 3 months | 85222 ± |
| | | (n = 7) | 22.4 | 10.2 | 15.5 | (n = 6) | 12.1 | | | (n = 5) | 71774 |
| | | 6 months | 62.6 ± | 33.8 ± | 3.5 ± | 6 months | 39.2 ± | | | 6 months | 93443 ± |
| | | (n = 9) | 10.7 | 8.2 | 3.3 | (n = 9) | 7.4 | | | (n = 5) | 37259 |
| | HEART | | | | | Dup 2 | 7.2 ± | | | | |
| | | | | | | (n = 4) | 6.3 | | | | |
| | | | | | | WT | 100 ± | | | | |
| | | | | | | (n = 2) | 28.8 | | | | |
| | | 1 month | 57.3 ± | 31.9 ± | 10.8 ± | 1 month | 45.7 ± | | | | |
| | | (n = 4) | 12.8 | 8.7 | 4.2 | (n = 5) | 8.2 | | | | |
| | | 3 months | 22.3 ± | 55.3 ± | 17.3 ± | 3 months | 36.4 ± | | | 3 months | 30641970 ± |
| | | (n = 7) | 14.1 | 15.2 | 8.2 | (n = 6) | 12.1 | | | (n = 5) | 67365591.0208669 |
| | | 6 months | 68.5 ± | 29 ± | 2.4 ± | 6 months | 58.3 ± | | | 6 months | 764899 ± |
| | | (n = 9) | 16.1 | 12.4 | 5 | (n = 9) | 9.8 | | | (n = 5) | 586562 |
| | DIA | | | | | Dup 2 | 6.2 ± | Dup2 | 73.1 ± | | |
| | | | | | | (n = 3) | 5.7 | (n = 3) | 1.5 | | |
| | | | | | | WT | 100 ± | WT | 1.6 ± | | |
| | | | | | | (n = 3) | 52.5 | (n = 4) | 0.2 | | |
| | | 1 month | 45.3 ± | 38.5 ± | 16.2 ± | 1 month | 136.3 ± | | | | |
| | | (n = 4) | 28.1 | 28 | 9.3 | (n = 5) | 61.9 | | | | |
| | | 3 months | 34.4 ± | 44.1 ± | 21.5 ± | 3 months | 117.4 ± | | | 3 months | 18169 ± |
| | | (n = 7) | 21.3 | 20.6 | 7.1 | (n = 6) | 45.1 | | | (n = 5) | 12491 |
| | | 6 months | 76.9 ± | 15.1 ± | 8 ± | 6 months | 85.2 ± | ACCA | 2.3 ± | 6 months | 45697 ± |
| | | (n = 9) | 13.6 | 6.8 | 8 | (n = 7) | 55.8 | (n = 9) | 1.3 | (n = 5) | 34463 |

### Example 6

### Early Systemic Delivery of scAAV9.U7-ACCA in Neonatal Dup2 Mice Results in Normal Muscle Structure, Shows No Off-Target Pathology, and Results in Functional Rescue of Muscle

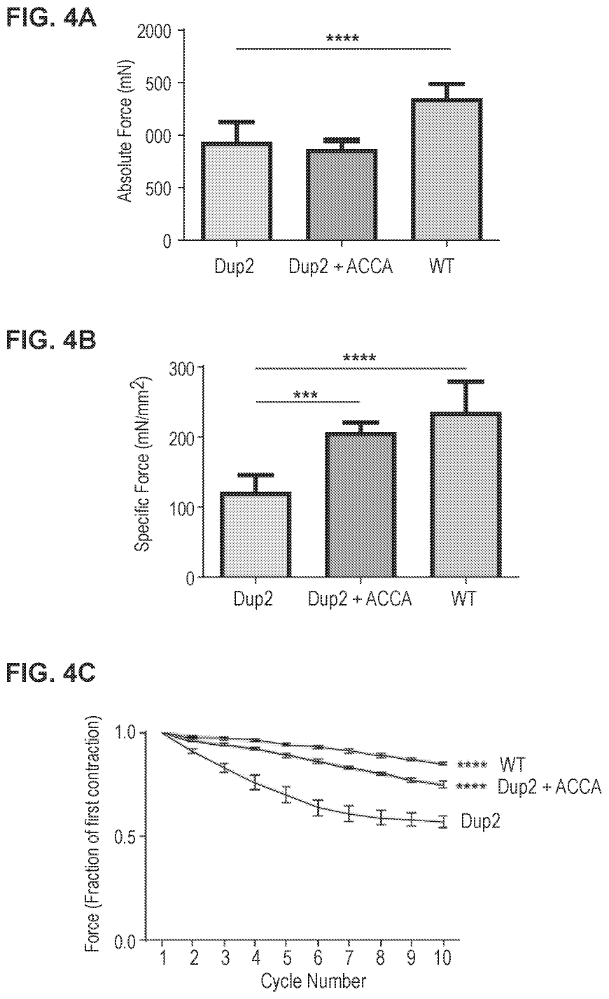

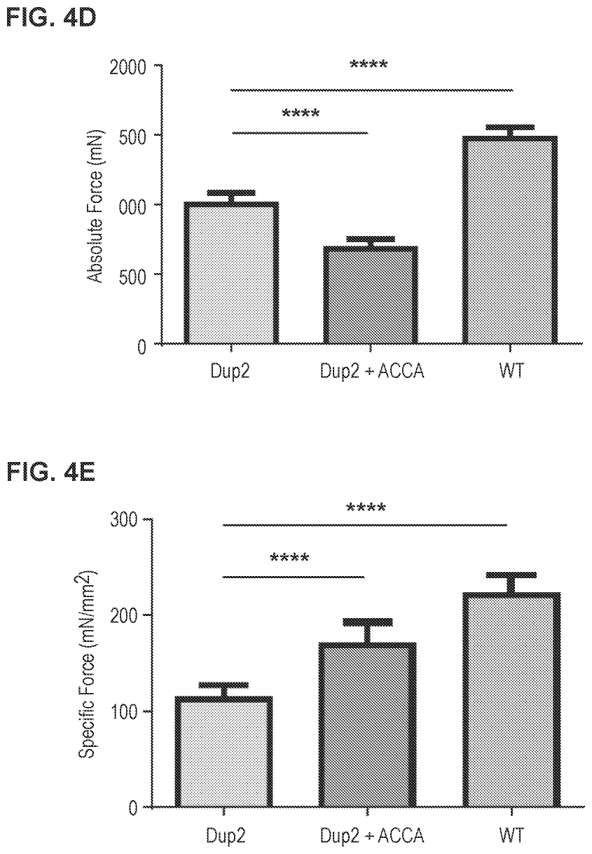

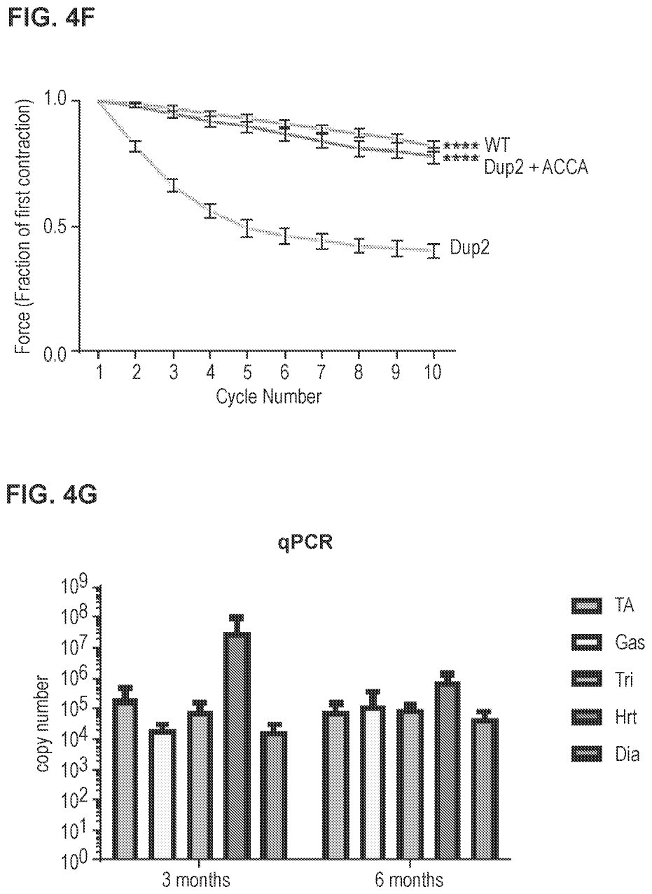

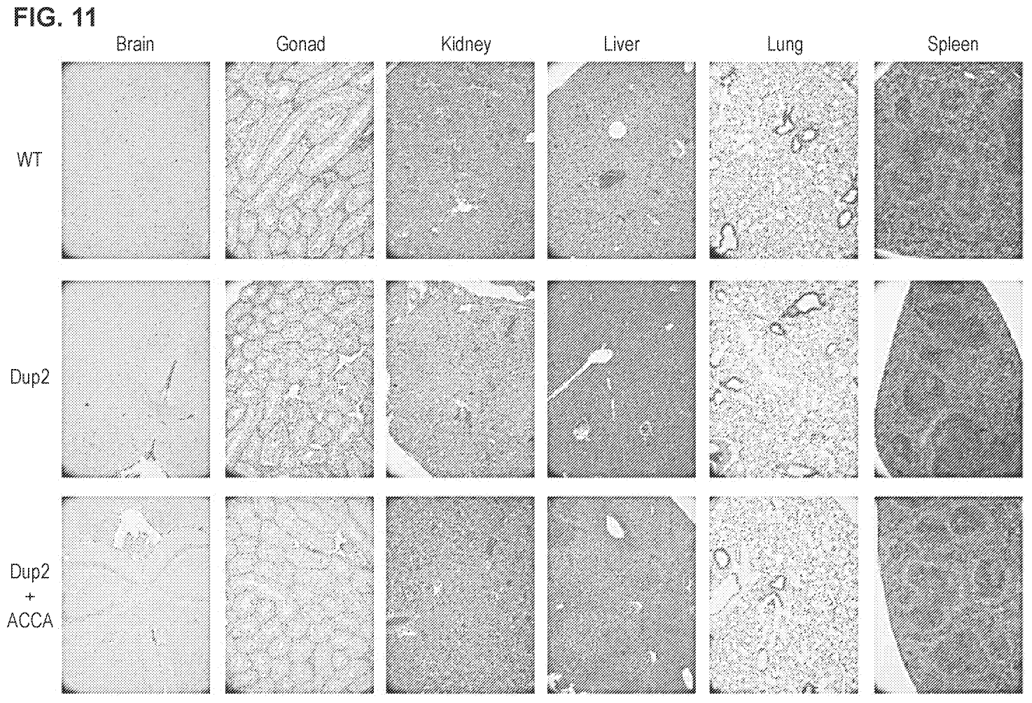

Muscle histologic analysis appeared normal, with no sign of necrosis or infiltration, and treated muscle revealed almost no central nucleation, indicating that the early treatment prevented muscle degeneration (FIGS. 3D-E, 9D-E, and 10D-E, and Table 4). Histopathologic analysis was performed on different non-muscle organs by a trained pathologist (Dr. Christopher Pierson), who reported that some livers occasionally had patchy small foci of inflammation that may contain neutrophils, but no signs of pathology were observed in other tissues up to 6 months post-injection (FIG. 11). Absolute force, as measured in the TA, did not increase at 3 and 6 months (FIGS. 4A and 4D), but specific force, as measured in the TA, improved (FIGS. 4B and 4E) with a complete rescue at both 3 and 6 months of the force drop following repeated eccentric contractions seen in untreated Dup2 mice (FIGS. 4C and 4F, and Table 2). Together, these data suggest that early systemic treatment with scAAV9.U7-ACCA prevents the muscle pathology and dysfunction characterizing muscular dystrophy.

### Example 7

### scAAV9.U7-ACCA Skipping of Exon 2 Results in Dystrophin Expression in Human Cells Harboring Non-Dup2 Exon 1-4 Mutations

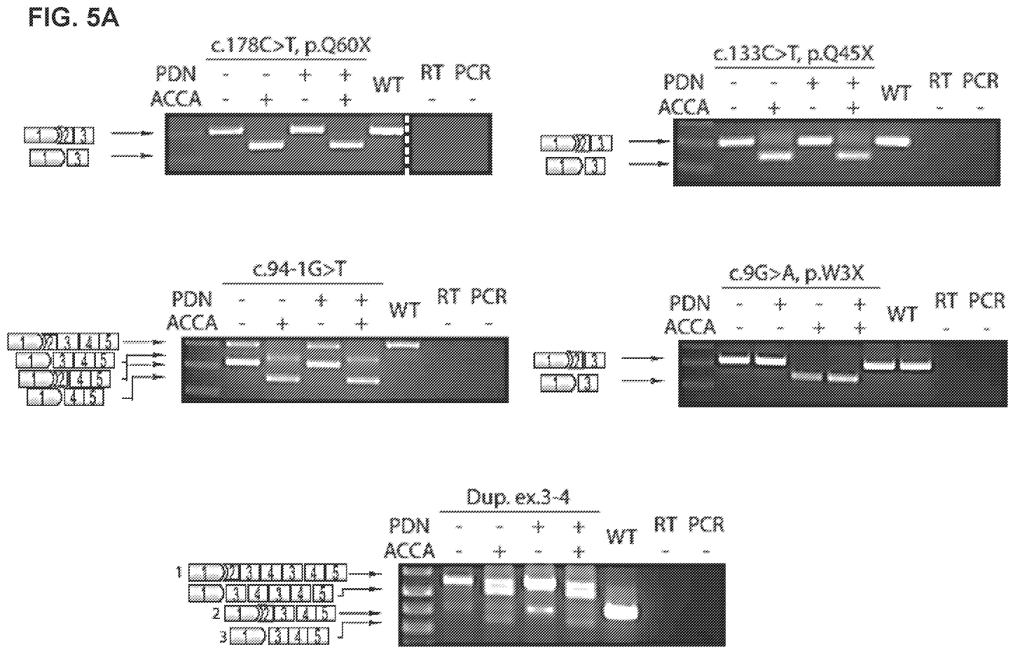

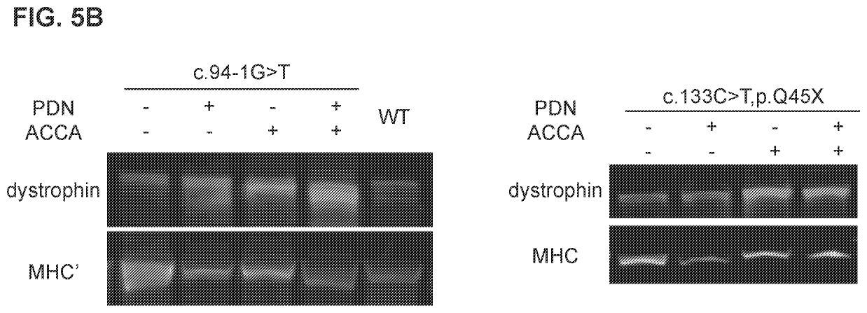

Mutations in exons 1 to 4 represent 5-6% of the DMD patient population (http colon-slash-slash www.dmd.nl). As complete skipping of exon 2 results in an out-of-frame transcript that is robustly translated by use of the exon 5 IRES (Wein et al. (2014), supra), it was hypothesized previously that scAAV9.U7-ACCA could represent a therapeutic strategy, not only for exon 2 duplication, but also for this patient population. Because no animal models are available to test this hypothesis, patient-derived fibroblasts that were transdifferentiated into myoblasts (FibroMyoD) were used to evaluate exon skipping and dystrophin expression following treatment with scAAV1.U7-ACCA, with or without PDN. The five cell lines each carried different mutations (FIG. 5A). Three cell lines have nonsense mutations within exons 1-3; one cell line carries a splice site mutation in the −1 position relative to exon 3 that results in an in-frame exon 3-deleted transcript; and one cell line carries an in-frame duplication of exons 3-4. After treatment, all cell lines with nonsense mutations displayed a transcript corresponding to nearly 100% exon 2 skipping. This transcript leads mainly to expression of the N-truncated but highly functional dystrophin. AAV transduction in the c.94-1G>T cells led to transcripts missing both exon 2 and exon 3, and in the exon 3-4 duplication cells led to two main bands that correspond to the non-skipped and exon 2-skipped transcripts along with two fainter bands corresponding to the wild-type and exons 2-3-4 skipped transcript. These two transcripts lead to expression of both the N-truncated but highly functional dystrophin and the full length dystrophin. Despite the difficulty of producing reliable dystrophin Western blot results from FibroMyoD cells, quantifiable blots for two cell lines were obtained (FIG. 5B). Without treatment, the c.94-1G>T line expressed low levels of dystrophin, but exon 2 skipping resulted in significantly increased dystrophin expression, suggesting that inducing an early premature termination codon results in IRES activation. The second cell line harbors an exon 3 nonsense mutation (c.133C>T, p.Q45X); it already expresses a low level of dystrophin, suggesting utilization of the IRES as we previously demonstrated with frame-truncating mutations in this region (Wein et al., (2014), supra), but increased dystrophin expression is seen with skipping of exon 2 (FIG. 5B).

Systemic delivery of scAAV9.U7-ACCA confirms the results from preliminary in vivo and in vitro studies of exon 2 skipping, in which both cell culture and intramuscular injection studies showed induction of dystrophin expression (Wein et al., (2015), supra). Extending those results, these experiments demonstrate the effectiveness of a one-time tail vein injection of scAAV9.U7-ACCA in either adult or neonatal Dup2 mice, resulting in both wild-type and Del2 transcripts. Both are therapeutic, as they result in translation into either full-length wild-type dystrophin, or the highly functional IRES driven dystrophin. In adult mice, this treatment led to improvements of muscle function and amelioration of the dystrophic phenotype by 3 months post-injection. In neonatal mice, a single injection at PO-P1 restored dystrophin expression and prevented pathologic and physiologic features in the Dup2 mouse for at least 6 months, consistent with the high degree of exon skipping efficiency demonstrable over the same period.

Levels of dystrophin as low as 3.2% of normal can significantly ameliorate disease symptoms in dystrophinopathy patients (Waldrop et al., Neuromuscul Disord 28, 116-121 (2018)). Treatment with scAAV9.U7-ACCA consistently resulted in robust dystrophin expression. For example, in the treated Dup2 mice, levels of dystrophin reached a minimum of 40%, well above commonly cited values of 5-20% described as being found in BMD (Aartsma-Rus et al., J Neuromuscul Dis 6, 147-159 (2019)). Moreover, the treatment described herein resulted in significantly greater levels of expression of the IRES-driven dystrophin isoform compared to the c.9G>A founder allele mutation, which results in dystrophin levels of 5-15% of normal, associated with ambulation into the seventh or eighth decade (Gurvich et al., Hum Mutat 30, 633-640 (2009)). Inducing expression of these full-length and/or IRES-driven dystrophins resulted in significant improvements of muscle function. Notably, the muscle with the greatest degree of dystrophin restoration, i.e., expression showing nearly 100% of wildtype, was the diaphragm. Such finding was very significant given that respiratory failure is the most common cause of death in DMD.

Ongoing and recently completed clinical trials have validated the safety profile of AAV9 mediated gene therapies. In one such study, patients as young as 9 months old were injected with AAV9 carrying the SMN transgene, a gene mutated in spinal muscular atrophy (SMA), a fatal neuromuscular disorder (Mendell et al., N Engl J Med 377, 1713-1722 (2017)). Results show little to no serious side effects, and remarkable patient survival, leading to FDA approval for commercial use of the vector in SMA patients. The safety profile of the AAV9 construct described in the experiments herein was assessed by a blinded, non-good laboratory practice (non-GLP) toxicological study performed in Dup2 mice. Review by a pathologist (C.P.) of histopathological sections of the brain, gonad, kidney, liver, lung, and spleen of treated and untreated mice found no difference between both groups (FIG. 11), supporting the tolerability of this treatment.

The experiments described herein above tested the hypothesis that inducing exon 2 skipping would result in exon 5 IRES activation and would be of benefit to the 5-6% of DMD patients having a mutation within exons 1-4, and it was found that delivery of the vector led to efficient exon skipping in 5 patient cell lines, and to production of dystrophin in those cell lines which could be successfully maintained in culture long enough to express dystrophin. Notably, both of these cell lines with in-frame transcripts already expressed low levels of dystrophin, consistent with the patients' severe BMD phenotypes, and presumably due to the known instability of proteins with N-terminal variation (Henderson et al., Proc Natl Acad Sci USA 107, 9632-9637 (2010)).

This study did not differentiate the 427kD wild-type dystrophin form versus the 413 kD IRES-driven form of dystrophin, as reproducible separation by Western blot is problematic. Nevertheless, skipping of exon 2 results in both forms; and, as the patients expressing dystrophin from the c.9G>A allele demonstrates, both forms are therapeutic. At the RNA level, RT-PCR (FIG. 1A and FIG. 3A) experiments demonstrate at least 60% presence of the Del2 transcript, suggesting that most of the protein expressed corresponds to the IRES-initiated dystrophin. Notably, the presence of this isoform is able to protect muscle from contraction-induced injury and correct muscle force to near control levels, despite missing half of the canonical actin binding domain 1 (ABD1), as translation beginning in exon 6 results in a protein lacking the first of two calponin homology domains (CH1 and CH2).

Our results demonstrate the robustness, durability, and safety of exon skipping following delivery of scAAV9.U7snRNA.ACCA. Thus, treatment with scAAV9.U7-ACCA is a potential therapy for 5-6% of all DMD patients. These experiments demonstrate not only a potential treatment for patients with a duplication of exon 2, but a potential strategy for creating treatments for many other DMD mutations and other neuromuscular disorders.

### Example 8

### Determination of Optimal Dosing

Preclinical studies were conducted with material that was originally titered using a qPCR method, involving a supercoiled standard and a primer/probe set that is found in the vector genome four times (shown as “U7 probe (4 copies)” in Table 5 below). Additional testing was carried out using optimal density (OD) and the titer was adjusted for the preclinical lot, referred to as the “OD” titer. Based off of the OD titer, the minimum effective dose (MED) was determined to be 1.0 E14 vg/kg. In Table 5, “Vector Specific Probe” refers to the primers and conditions described in Example 1, paragraph 100.

The viral vector core (VVC) and Clinical Manufacturing Facility (CMF) updated their titering methods to utilize a linear standard in the qPCR. Because titering methods were changing, new primer/probe sets were designed so that they are present in the genome only once. These are described in Example 1 and refer to the “Vector Specific Probe” in Table 5. The MED was calculated to be about 2.9 E13 vg/kg, which was rounded up to 3e13 vg/kg.

Two conversions were carried out. The conversion rate from the OD titer to the historical VVC titer (supercoiled standard, and primer/probe set present 4 times) was determined to be an increase of 1.8×.

$$1. E 14 vg / kg ( Thesis MED ) \times 1.8 = 1.8 E 14 vg / kg$$

The conversion rate from the historical VVC titer (supercoiled standard, and primer/probe set present 4 times) to the updated VVC/CMF titer (linear standard, unique primer/probe set) was carried out in a titer comparison study in the CMF and was determined to be a decrease of 6.12×.

$$1.8 E 14 vg / kg / 6.12 = 2.9 E 13 vg / kg ( Clinical MED )$$

TABLE 5
Summary Table.

| Preclinical Efficacy (IV Dose Escalation) | | | Proposed |
|---|---|---|---|
| | U7 probe (4 | Vector Specific | Clinical Doses |
| | copies) | Probe | Vector Specific |
| Dose Based | Dose Based on | Dose Based on | Probe |
| on Nominal | Nominal | Linearized Standard | Linearized |
| OD Titer | Supercoiled Titer | Equivalent Titer | Standard Dose |
| (vg/kg) | (vg/kg) | (vg/kg) | (vg/kg) |
| 2.6E+14 | 4.7E+14 | 7.6E+13 | 8.0E+13 vg/kg |
| 1.0E+14 | 1.8E+14 | 2.9E+13 | 3.0E+13 vg/kg |
| 3.2E+13 | 5.8E+13 | 9.4E+12 | |
| 1.0E+13 | 1.8E+13 | 2.9E+12 | |
| 3.2E+12 | 5.8E+12 | 9.4E+11 | |
| 1.0E+12 | 1.8E+12 | 2.9E+11 | |

OD to supercoiled titer (4
copies) is increase of 1.8x

Titer comparison report from
supercoiled (4 copies) to linear
(1 copy) is reduction of 6.12x

### Example 9

### A 3-Month and 6-Month Toxicity Study of Single Dose of scAAV9.U7.ACCA by Intravenous Infusion in Male Juvenile Cynomolgus Monkeys

The objective of this study was to determine the potential toxicity of scAAV9.U7.ACCA when given once on Day 1 by intravenous (IV) infusion to male juvenile cynomolgus monkeys. The data provided herein reflect data from an Interim Report of a toxicity study of single dose of scAAV9.U7.ACCA by intravenous infusion in male juvenile cynomolgus monkeys. Time points, procedures, and results described herein this section applied to all the animals through Day 91. Applicable time points and procedures through Day 182 are described herein, but the additional results will be included in a Final Report, and are not described in this example. The study design was as follows:

TABLE 6
Experimental Design.

| | | | | | No. of Males | |
|---|---|---|---|---|---|---|
| | | | Dose | Dose | 3 month | 6 month |
| Group | | Dose Level | Volume^{a} | Concentration | Phase | Phase |
| No. | Test Material | (vg/kg/d) | (mL/kg) | (vg/mL) | (Arm A) | (Arm B) |
| 1 | Control Article | 0 | 8 | 0 | 2 | 1 |
| 2 | scAAV9.U7.ACCA | 3 × 10¹³ | 8 | 0.37 × 10¹³ | 2 | 1 |
| 3 | scAAV9.U7.ACCA | 8 × 10¹³ | 8 | 1 × 10¹³ | 2 | 1 |

^{a}The duration of infusion was targeted at 30 minutes.

### Methodology

The test and control articles were administered to the appropriate animals (Groups 1 to 3) via IV infusion into a suitable peripheral vein once on Day 1 using a calibrated infusion pump (target 30 minutes infusion). The actual start and stop times of dose administration were recorded in the study record. A hemostat clamp was placed on the infusion line after dose completion and dosing syringes were delivered in vertical position to the Formulations Laboratory for collection of dose formulation analysis samples. The dose volume for each animal was based on the most recent body weight measurement. The animals were temporarily restrained for dose administration and were not sedated. The first day of dosing was designated as Day 1.

The IV route of exposure was selected because this is the intended route of human exposure. Dose levels were based on prior studies that demonstrated the minimal efficacious dose (MED) in Dup2 transgenic mice was about 3×10¹³ vg/kg/d. The MED and a dose higher (i.e., 8×10¹³ vg/kg/d) were evaluated to provide a safety margin for clinical dosing in patients. The test article, scAAV9.U7.ACCA, was provided at a concentration of 2.18×10¹³ vg/mL.

The in-life procedures, observations, and measurements listed below were performed for all animals. Throughout the study, animals were observed for general health/mortality and moribundity at least once daily in the afternoon. Animals were not removed from the cage during observation, unless necessary for identification or confirmation of possible findings.

The following parameters and end points were evaluated in this study: clinical signs, body weights, qualitative food consumption, ophthalmology, echocardiograms, neurologic examinations, clinical pathology parameters (hematology, coagulation, clinical chemistry, urinalysis, troponin I, and cardiac biomarkers), bone marrow cytology, bioanalytical parameters, tissue biodistribution quantitative polymerase chain reaction (qPCR) analysis, PBMC enzyme-linked immunospot (ELISpot) analysis, RNAseq analysis, immunohistochemistry, gross necropsy findings, organ weights, and histopathologic examinations.

### Results

There were no scAAV9.U7.ACCA-related effects on the following: clinical signs, body weights, qualitative food consumption, ophthalmology, neurological examinations, cardiac biomarkers, urinalysis parameters, bone marrow cytology, gross necropsy, or changes in absolute or relative organ weights.

Total DMD (Dystrophin) mRNA levels decreased in a dose-dependent manner in all muscle tissues (heart, quadriceps, diaphragm), reaching up to 38% reduction in expression in the hearts of animals treated with 8×10¹³ vg/kg/d of scAAV9.U7.ACCA. Such a decrease is entirely expected in normal NHPs, because a deletion of exon 2 is not present, and skipping of exon 2 only results in a transcript missing exon 2, which results in less efficient protein translation than wild-type transcript. By removing exon 2 in a wild-type context, it was expected that the stability of the mRNA would decrease. DMD expression was relatively stable in the liver and testis tissues at both doses (and vehicle control). Exon 2 skipping followed a similar dose dependence in the muscle tissues. Exon 2 skipping was most pronounced in the hearts of animals treated with 8×10¹³ vg/kg/d of scAAV9.U7.ACCA with 67% skipping.

There were no remarkable echocardiography findings in any animal. The decrease in right ventricular internal diameter in diastole (MM) in Group 2 or 3 of 34% and 29%, respectively, was assessed as of uncertain significance.

scAAV9.U7.ACCA-related changes in hematology parameters were limited to minimally increased monocytes and large unstained cells at both dose levels on Day 7 and at 3×10¹³ vg/kg/d on Day 14, which were generally comparable to control and/or baseline by Day 28.

scAAV9.U7.ACCA-related changes in coagulation parameters were limited to minimally decreased fibrinogen at 8×10¹³ vg/kg/d on Days 56 through 84.

scAAV9.U7.ACCA-related changes in clinical chemistry parameters included mildly increased triglycerides at 8×10¹³ vg/kg/d between Days 7 to 70 with values that were comparable to control and/or baseline by Day 84. On Days 7 and 14, minimally to moderately increased alanine aminotransferase (ALT) occurred in one of three animals and two of three animals in the 3 and 8×10¹³ vg/kg/d groups, respectively, and aspartate aminotransferase (AST) was minimally increased in one animal at 8×10¹³ vg/kg/d on Day 7. Additionally, AST, ALT, and lactate dehydrogenase (LDH) were increased for 1 animal at 8×10¹³ vg/kg/d on Days 56 and 70.

scAAV9.U7.ACCA-related microscopic findings were observed in the liver at 3×10¹³ vg/kg/d and increased in incidence in a dose dependent fashion. Minimal single cell, hepatocyte necrosis was observed at 3×10¹³ vg/kg/d. In addition, mild, diffuse hepatocellular vacuolization was observed in one 8×10¹³ vg/kg/d dose group animal.

In conclusion, administration of scAAV9.U7.ACCA by a single intravenous infusion was well tolerated through Day 91 in cynomolgus monkeys at levels up to 8×10¹³ vg/kg/d. Minimal microscopic findings including single cell hepatocyte necrosis was observed in the liver at 3×10¹³ vg/kg/d. In addition, mild, diffuse hepatocellular vacuolization was observed in one 8×10¹³ vg/kg/d dose group animal. Transient increase in triglycerides (minimal to mild), alanine aminotransferase (moderate), and aspartate aminotransferase (moderate) were observed 3×10¹³ vg/kg/d. There were no remarkable echocardiography findings in any animal. The decrease in right ventricular internal diameter in diastole (MM) in Group 2 and 3 of 34 and 29%, respectively, was assessed as of uncertain significance.

### Example 10

### Intramuscular Dose Escalation Study with scAAV9.U7-ACCA

The purpose of this study was to determine the optimal dose of a self-complementary adeno-associated viral (scAAV) vector expressing an U7.snRNA (U7.ACCA) under the control of the mouse U7 promoter in Dup2 mice following a single intramuscular injection into the tibialis anterior (TA). Five single doses of the scAAV9.U7.ACCA test article at half-log increments: 2.0×10¹⁰ vg, 5.6×10¹⁰ vg, 2.0×10¹¹ vg, 5.6×10¹¹ vg and 2.0×10¹² vg were administered in the tibialis anterior (TA) muscle. Molecular and force analysis were performed 4 weeks post-injection.

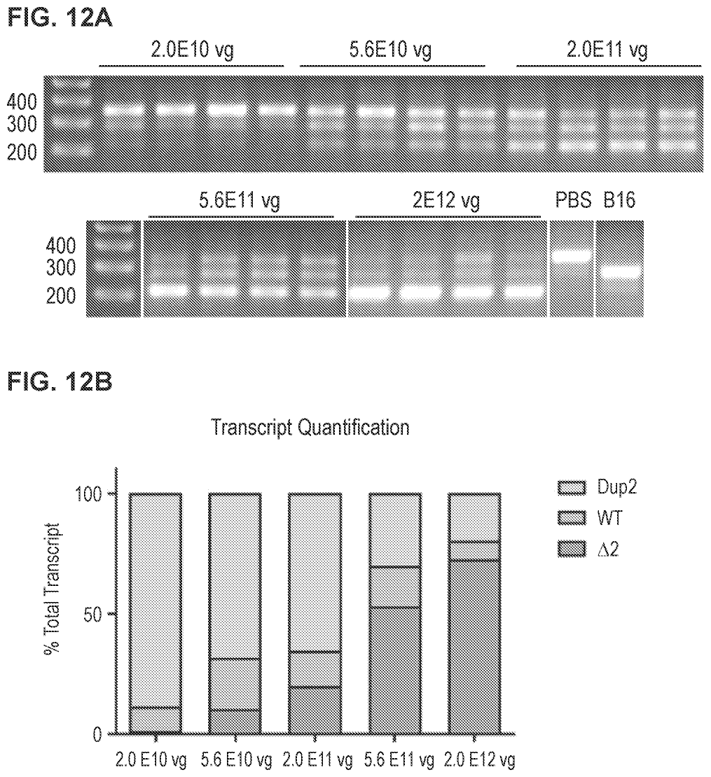

Each dose group consisted of 5-6 mice at 8-9 weeks of age, and in each mouse both TA muscles were injected and analyzed (n=10-12 muscles per dose group) at 4-5 weeks post-injection. For the physiology studies, an additional 6 mice were injected with the highest dose. RT-PCR was used to evaluate the amount of exon 2 skipping seen at the mRNA level, where potential transcripts include a duplication of exon 2 (Dup2), a single copy of exon 2 (WT), or zero copies of exon 2 (Δ2) (FIGS. 12A-B). The results show an expected dose response, with the lowest dose of 2.0×10¹⁰ vg resulting in 11% WT transcript, and the incremental dose of 5.6×10¹⁰ vg showing both the WT and 42 transcripts each comprising at least 10% of the total transcript. At the highest dose of 2.0×10¹² vg, only 20% of the transcript is Dup2, while the 42 transcript represents almost 75% of the total transcript (FIG. 12A-B).

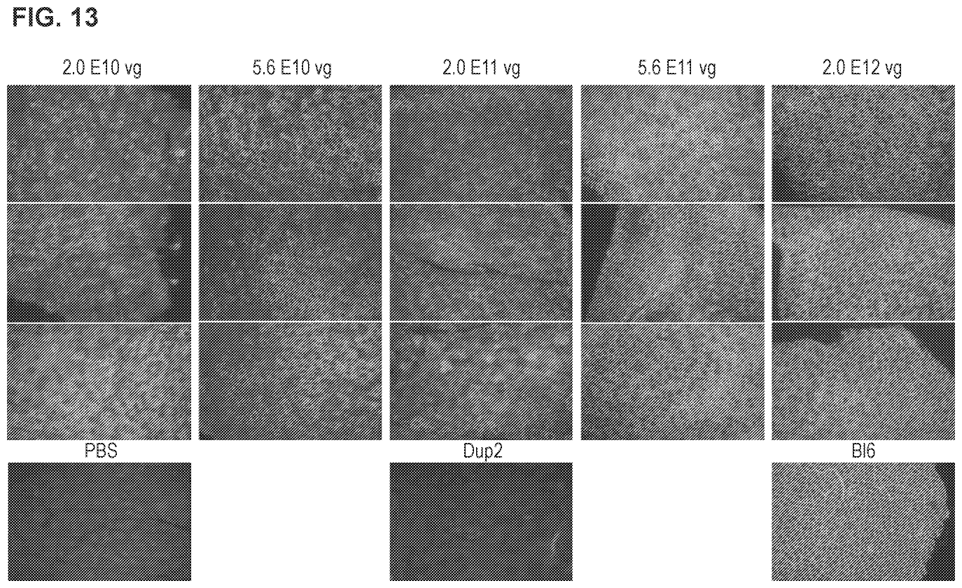

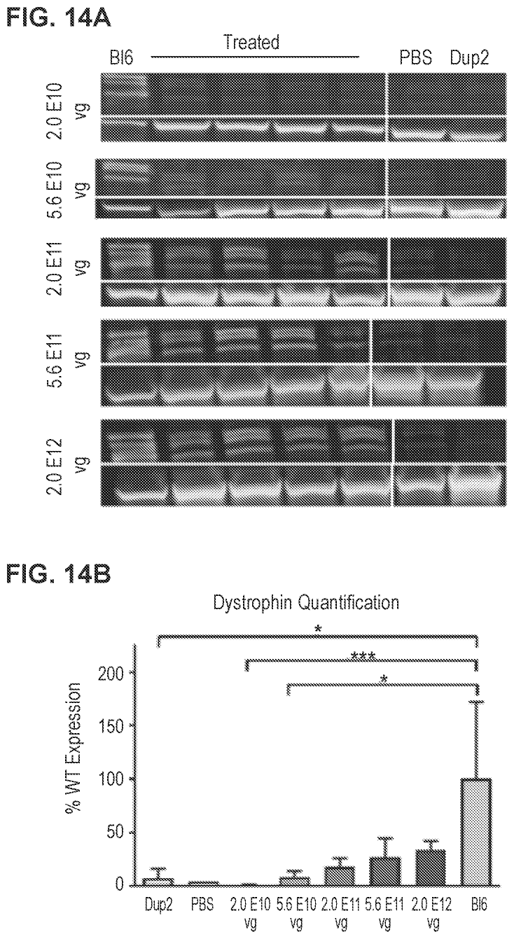

Dystrophin immunofluorescence (IF) analysis on frozen sections shows proper sarcolemmal localization at all five doses tested (FIG. 13). Staining can be seen at the lowest dose and increases with each successive dose, consistent with the RT-PCR analysis. Qualitatively, by 5.6×10¹¹ vg fiber membranes are almost completely stained and maintain this level of staining in the highest dose, 2.0×10¹² vg. Dystrophin protein was quantified by immunoblot (IB), confirming the dose response; at the highest dose, 2.0×10¹² vg, dystrophin restoration reaches almost 35% of the level seen in control animal muscle (FIG. 14A-B).

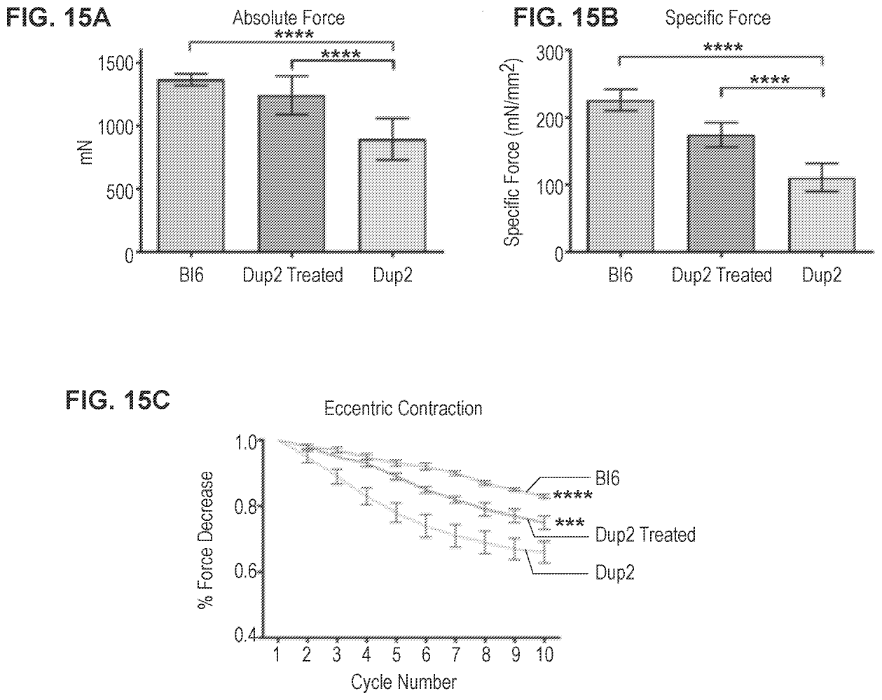

To determine the degree of functional rescue, in vivo physiology studies were performed on additional TA muscles four weeks after a single intramuscular injection of 2.0×10¹² vg, measuring both force (absolute and specific) and force drop following repeated eccentric contractions. Treated Dup2 muscle shows no statistically significant difference in absolute force in comparison to C57BL/6 mice, unlike uninjected Dup2 control muscle. Treatment results in a partial rescue of specific force, with statistically significant improvement in comparison to untreated Dup2 (FIG. 15A and 15B). The Dup2 mouse is generally larger than control C57BL/6 mice 6, and this difference in size may account for the discrepancy between recovery of the absolute and specific force, as specific force takes into account muscle length and mass. Similarly, treated Dup2 muscles show a partial rescue of force drop following repeated eccentric contractions (FIG. 15C).

To assess the efficacy of scAAV9.U7.ACCA delivered intramuscularly into the TA, a study was designed including a total of 53 (45 Dup2 and 8 C57BL/6) animals. Of the 53 animals, 32 were treated with scAAV9.U7.ACCA at either 2.0×10¹⁰vg, 5.6×10¹⁰vg, 2.0×10¹¹vg, 5.6×10¹¹vg, or 2.0×10¹²vg and the remaining were either un-injected or PBS injected controls.

In this study, a dose-dependent effect of efficient skipping of either one or two copies of the duplicated exon 2 was demonstrated. Titration of the dose to skip only one copy of exon 2 was not achieved, suggesting a high efficiency for U7snRNA-mediated skipping. While skipping of a single copy of exon 2 restores wild-type protein, skipping both copies is not detrimental because it activates the IRES and allows for the production of a highly-functional dystrophin protein.

This skipping of exon 2 corresponds with a dose-dependent increase in properly localized dystrophin in response to treatment with the scAAV9.U7.ACCA test article as evidenced by immunofluorescence and Western blotting. Regardless of the isoform expressed following treatment with scAAV9.U7.ACCA in the TA of treated Dup2 animals, a complete restoration of absolute force was shown, as well as a partial rescue of both specific force and resistance to force drop following repeated eccentric contractions, mirroring the protection that the N-truncated isoform confers on subjects/patients.

In summary, while skipping of only one copy of exon 2 was not feasible in this study using the scAAV9.U7.ACCA test article, the treatment will not be detrimental to subjects/patients because skipping one or both copies of exon 2 results in restoration of dystrophin protein and, importantly, function.

### Example 11

### Intravenous Dose Escalation Studies with scAAV9.U7-ACCA

The purpose of these studies was to determine the minimally efficacious dose (MED) for a single systemic delivery of a self-complementary adeno-associated viral (scAAV) vector expressing an U7.snRNA (U7.ACCA) under the control of the mouse U7 promoter in Dup2 mice, and second to determine the effect of combinatorial treatment of scAAV9.U7.ACCA and 6-methyl-prednisolone (PDN).

Toward an ultimate goal of systemic delivery to reach all skeletal muscles, heart, and diaphragm, systemic intravenous (IV) dose escalation studies were carried out in order to establish a possible minimal efficacious dose (MED) necessary for designing IND-enabling toxicology studies, as well as for eventual clinical translation. Single tail vein injections of six ascending doses (1.8×10¹² vg/kg, 5.8×10¹² vg/kg, 1.8×10¹³ vg/kg, 5.8×10¹³ vg/kg, 1.8×10¹⁴ vg/kg, or 4.7×10¹⁴ vg/kg) of scAAV9.U7.ACCA were carried out in 8-9 week old Dup2 mice (n=3 per group). Necropsy was performed 4 weeks later, and TA, gastrocnemius, triceps, diaphragm and heart were removed for analysis of exon 2 skipping by RT-PCR, and dystrophin expression by immunofluorescence (IF) and Western blot (WB) analysis.

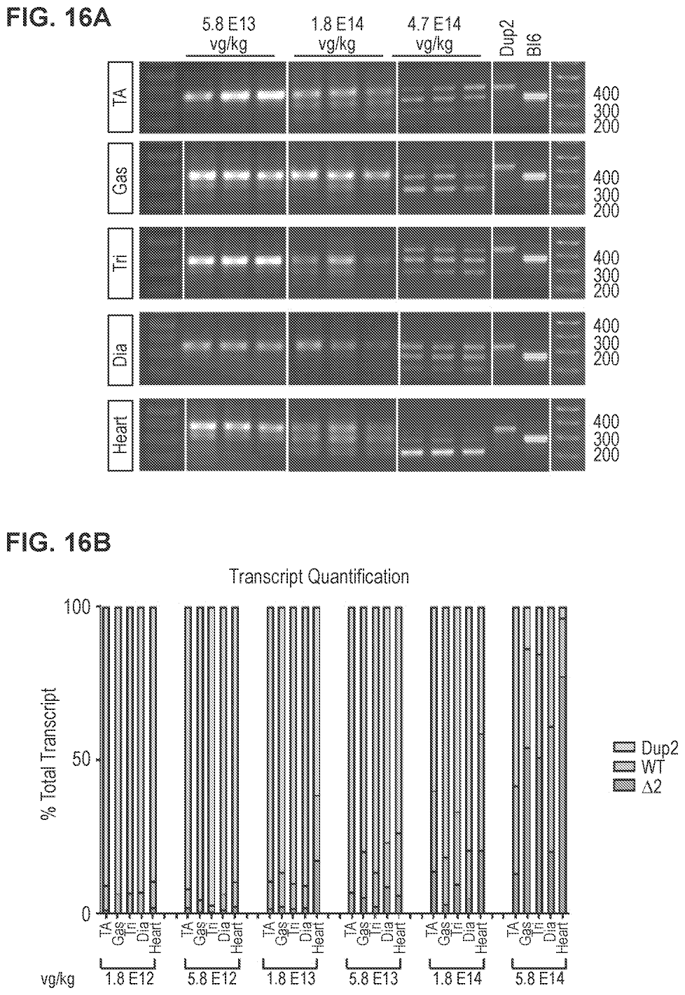

Quantification was performed for the Tibialas Anterior (TA), Gastrocnemius (Gas), Triceps (Tri), Diaphragm (Dia) and Heart at each dose. RT-PCR analysis of exon 2 exclusion shows the Dup2 transcript was the major species accounting for at least 60% of the total transcript at the lowest doses, i.e., doses between 1.8×10¹² vg/kg and 1.8×10¹³ vg/kg (FIGS. 16A-B). Higher doses, ranging from 5.8×10¹³ vg/kg to 4.7×10¹⁴ vg/kg, were associated with increased skipping of either one or two copies of exon 2 in at least 40% of the total transcript in all muscles at the highest dose of 4.7×10¹⁴ vg/kg. Notably, either the WT or A2 transcripts are therapeutic, given the presence of the dystrophin exon 5 IRES.

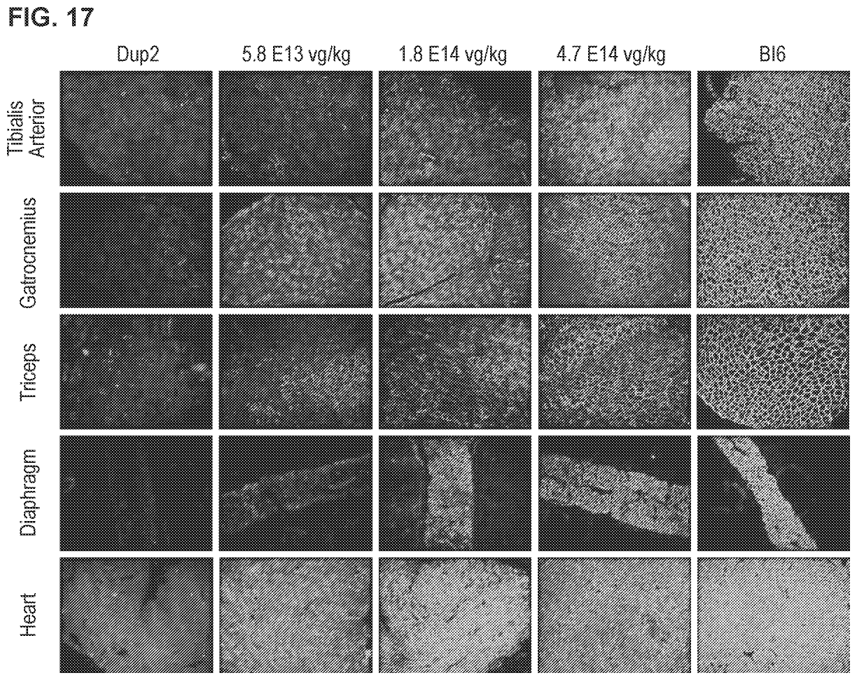

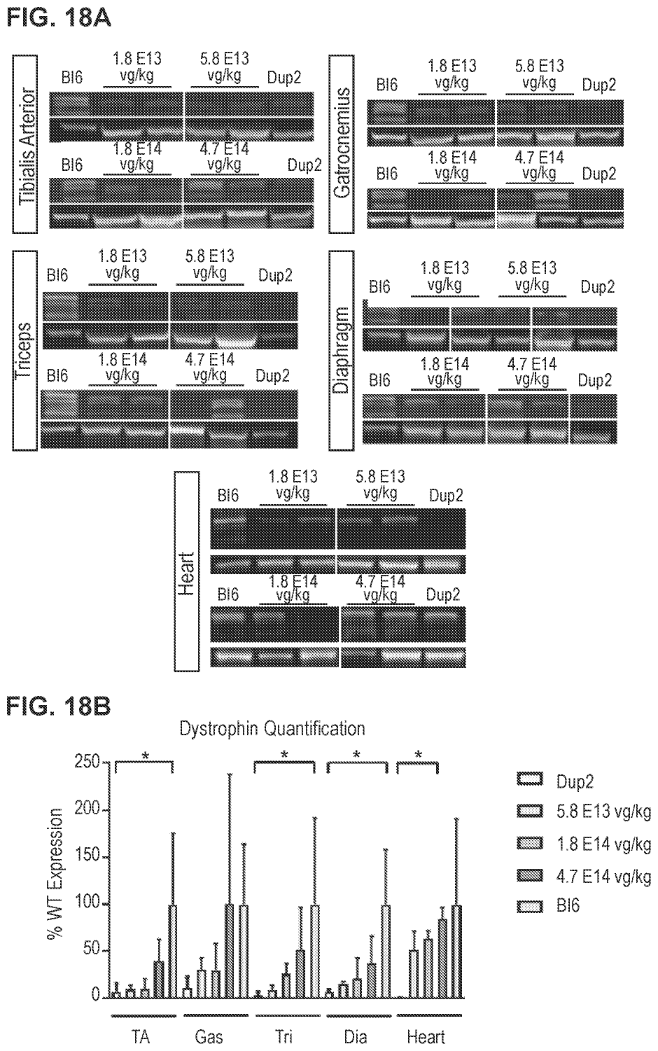

Immunofluorescence staining of dystrophin (dys) was performed on all tissue samples to determine the level of protein restoration. Representative immunofluorescence images are shown in FIG. 17. No significant dystrophin staining was seen at the three lowest doses (1.8×10¹² vg/kg, 5.8×10¹² vg/kg and 1.8×10¹³ vg/kg), except for the occasional revertant fibers (images not shown). Higher doses result in properly localized dystrophin at the sarcolemmal membrane, with the heart and gastrocnemius showing the greatest dystrophin signal (FIG. 17). At the highest dose, 4.7×10¹⁴ vg/kg, dystrophin is nearly completely restored in the heart and triceps. Quantification of dystrophin expression by immunoblot shows a dose-related increase in dystrophin protein, with levels in the gastrocnemius and heart reaching 100% and 85% that of WT muscle at 4.7×10¹⁴ vg/kg (FIG. 18A-B), consistent with the RT-PCR and immunofluorescence results. Staining showed proper sarcolemmal localization of the dystrophin protein at all doses when present with the heart and gastrocnemius showing the greatest dystrophin signal. Staining was not seen at the lowest dose except for the occasional revertant fiber but gradually increased with each successive dose. The heart is the first muscle to show dystrophin expression and remains one of the highest transduced muscles throughout each dose.

Protein was isolated from 5 muscles (TA, Gas, Triceps, Diaphragm and Heart) from each mouse and quantification was performed using Western blots on the three highest doses (5.8×10¹³, 1.8×10¹⁴ or 4.7×10¹⁴ vg/kg) following 4 weeks and 12 weeks of treatment. Dystrophin expression increased in a linear dose-dependent response in each muscle for both time-points. Following 12 weeks of therapy, Western blot analysis corroborated the immunofluorescence results and demonstrated a restoration of 90% wild type levels in the heart, 108% in the triceps, and 65% in the diaphragm.

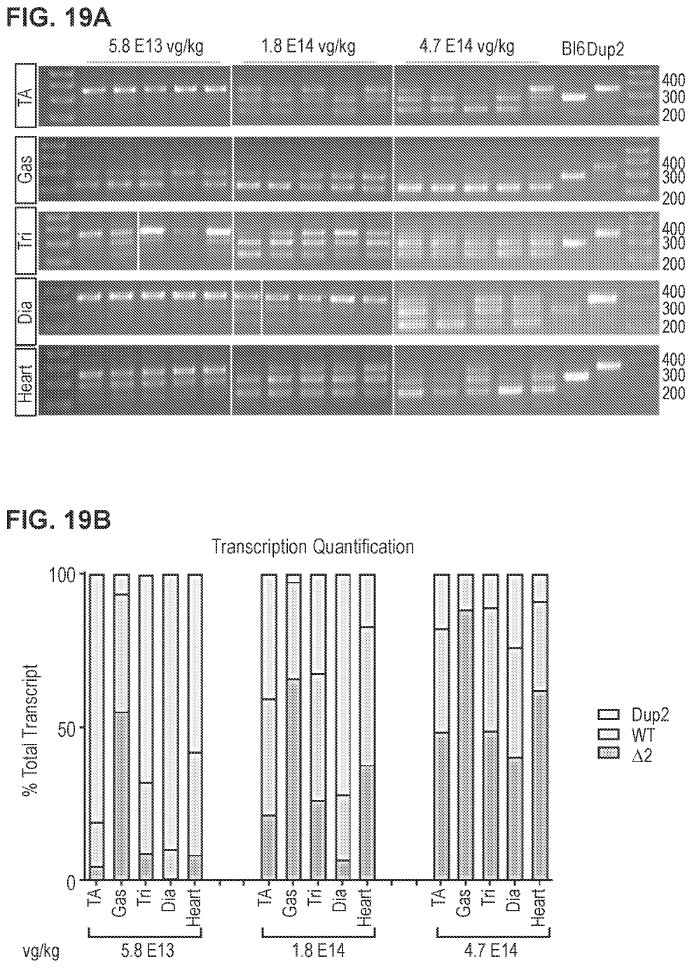

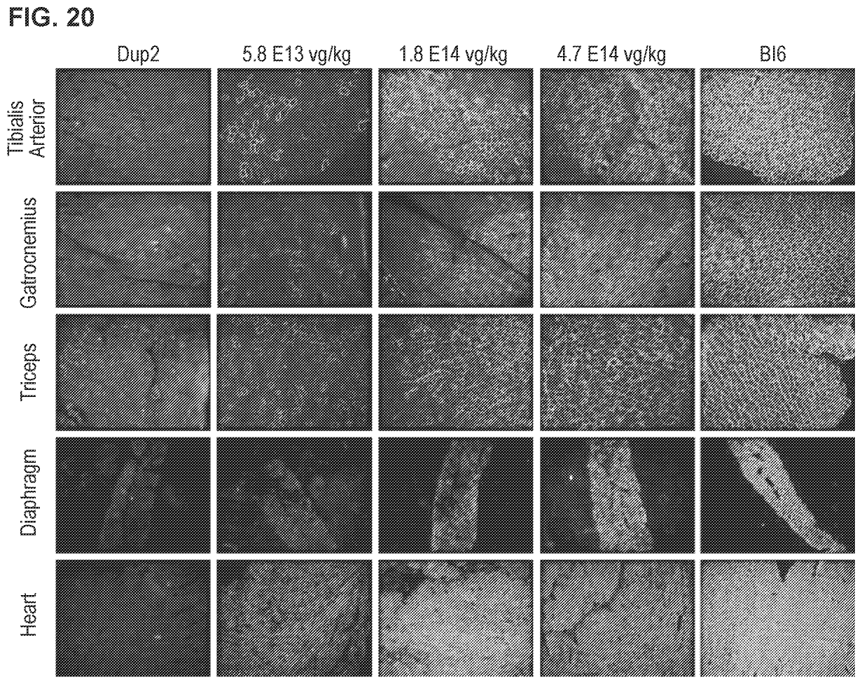

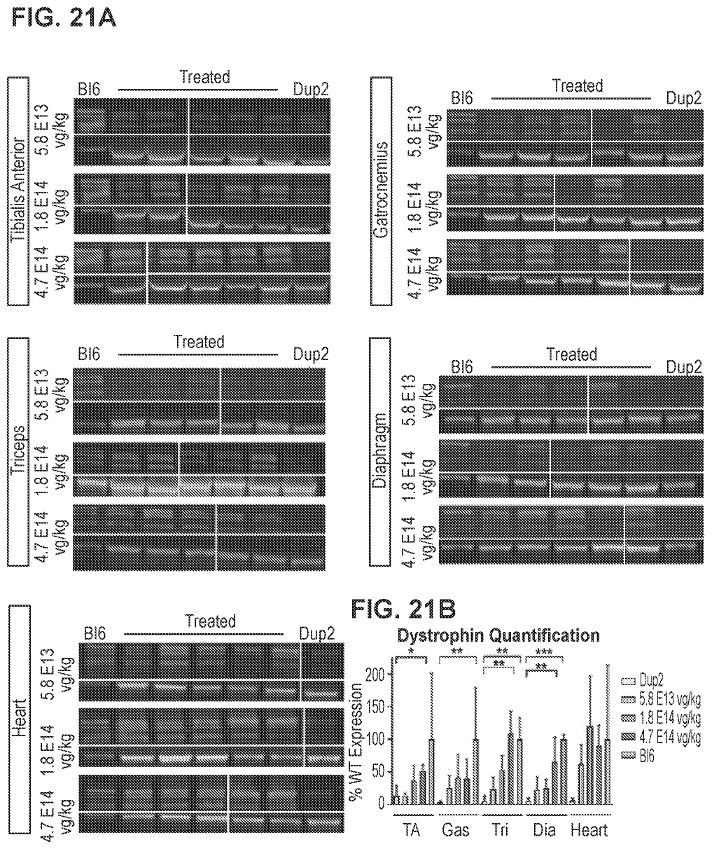

In order to further define a clinically relevant minimal efficacious dose, and to assess the time course of dystrophin expression, a second dose escalation was performed to show exon skipping in Dup2 mice at the three highest doses (5.8×10¹³ vg/kg, 1.8×10¹⁴ vg/kg or 4.7×10¹⁴ vg/kg) following 12 weeks of treatment. Exon 2 skipping, dystrophin expression, and muscle electrophysiology were analyzed. RT-PCR analysis (FIG. 19A-B) showed increased exon skipping at all doses, with single or double-skipping of exon 2 representing at least 75% of the total transcript at the highest dose of 4.7×10¹⁴ vg/kg. Immunofluorescence shows an increase in dystrophin at the lowest dose of 5.8×10¹³ vg/kg and nearly complete restoration of sarcolemmal dystrophin at the highest dose in all muscles (FIG. 20). Immunoblot analysis again corroborates the immunofluorescence results, showing levels of 90% of WT muscle in the heart, 108% in the triceps, and 65% in the diaphragm at the highest dose of 4.7×10¹⁴ vg/kg (FIG. 21A-B).

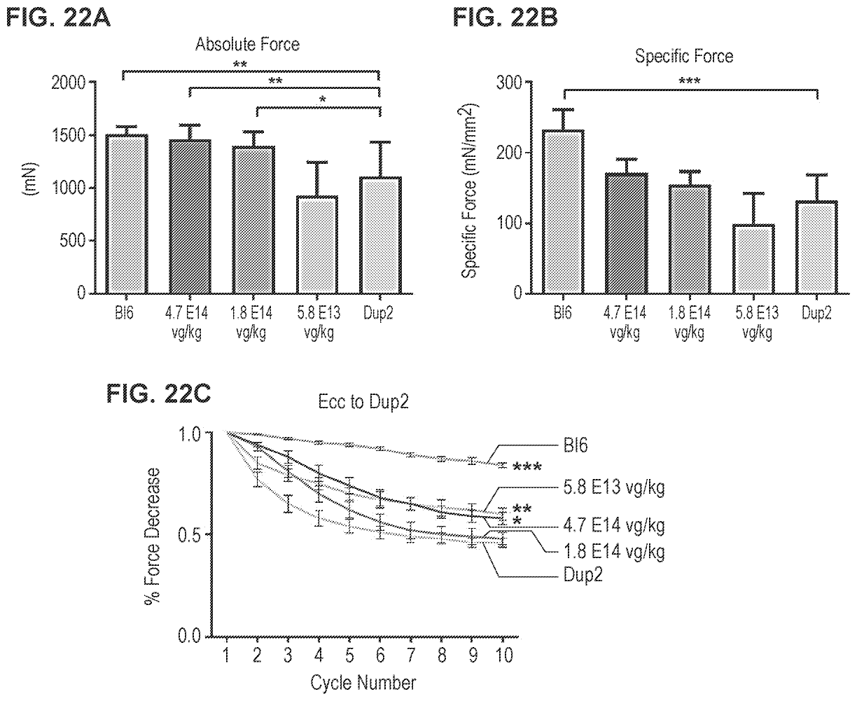

Electrophysiology studies were performed on the TA muscle of Dup2 mice treated with the three highest doses (5.8×10¹³ vg/kg, 1.8×10¹⁴ vg/kg, or 4.7×10¹⁴ vg/kg) and then mice were sacrificed at 12 weeks post-injection. For both absolute and specific force, a dose escalation curve was seen for the highest dose of 4.7×10¹⁴ vg/kg. Treatment at the two highest doses resulted in complete correction of absolute force to levels seen in C57BI/6 mice (control) (FIG. 22A), with partial correction of both specific force and force drop following repeated eccentric contractions (FIGS. 22B and 22C).

Dose escalation studies of both intramuscular and systemic delivery of scAAV9.U7.ACCA (presented herein in this Example and in the above Examples) showed efficient skipping of exon 2, along with increased expression of properly localized dystrophin that restores muscle function. These data indicate that skipping of a duplicated exon 2 is a feasible therapeutic approach, particularly because skipping of exon 2 may be associated with an apparently unlimited therapeutic window due to utilization of the IRES with complete exon 2 exclusion, and suggest that patients harboring other mutations 5′ of the exon 5 IRES may benefit from expression of this highly functional dystrophin isoform via the same mechanism.

These experiments show that systemic delivery of scAAV9.U7.ACCA induces robust skipping of exon 2 within DMD mRNA in skeletal muscle, heart, and diaphragm of Dup2 mice. This skipping results in two transcripts consisting of the wild-type mRNA, and an mRNA in which no copies of exon 2 are included (the Δ2 transcript). Both transcripts would be therapeutic because translation of the Δ2 mRNA (via utilization of the DMD IRES) results in a highly functional isoform of approximately 413 kD which, despite a partial N-terminal deletion, is associated with either a very mild BMD or with an essentially asymptomatic state.

The fact that the IRES is utilized in the absence of exon 2 makes the prospect of therapeutic exon 2 skipping particularly attractive due to the large therapeutic window, as “overskipping” results in the therapeutic Δ2 transcript. This is in contrast to out-of-frame duplications of exons within the central rod mutational hotspot of DMD which are typically associated with DMD, as predicted by the reading frame rule. In that region, only the single exon copy wild-type transcript would be therapeutic, while total exclusion of the exon would be expected to result in an out-of-frame deletion transcript that cannot be translated.

Clinically, a DMD patient with a duplication in this region could be expected to be made no worse by “overskipping,” as both the exon-duplicated and deleted transcripts truncate the reading frame. Exon 2 skipping, as measured by total therapeutic transcripts, corresponds with a dose-related increase in levels of dystrophin, as quantified by immunoblotting and assessed qualitatively by immunofluorescence, which also shows proper localization of dystrophin. The inference from the mRNA studies is that the protein recognized by the C-terminally directed antibody is expected to be both wild type and N-deleted isoforms, and it is unfortunate that the only antibody that might distinguish the presence of exon 1 specific epitopes translated from the wild-type mRNA is a mouse monoclonal that has proven inadequately sensitive to distinguish differential expression on mouse frozen sections. Regardless of the isoform expressed, systemic delivery of scAAV9.U7.ACCA results in complete restoration of absolute force, as well as a partial rescue in both specific force and force drop following repeated eccentric contractions, at least in the TA muscle, given the protection that expression of the N-deleted isoform confers on patients who express it as a founder allele (Flanigan et al., Neuromuscul. Disord. 2009; 19: 743-8).

Although RT-PCR and dystrophin expression following IM injection is useful in measures of potency for comparing vectors or vector lots, successful systemic delivery is critical to therapeutic translation, given that muscle represents 30-40% of adult body mass. Transduction and dystrophin correction in the heart and diaphragm are critical, as most DMD patients die of cardiac or respiratory insufficiencies. In the dose escalation studies presented herein, expression of dystrophin in the heart in particular seemed even more robust than in skeletal muscle, at comparable doses.

Comparison of the 12 week vs 4 week post-injection studies shows that both exon skipping and dystrophin expression are more robust at the later time point. These data suggest that as the expression of dystrophin protein occurs, the stability of the resultant wild-type and N-deleted isoforms results in their continued accumulation at the sarcalemmal membrane at longer time points.

Taken together, these experimental results suggest that systemic delivery of scAAV9.U7.ACCA is feasible. The data also indicate a minimal efficacious dose of 3×10¹³ vg/kg results in robust expression of full-length or near-full-length dystrophin isoforms. These data provide a solid foundation for the design of a clinical dosing regimen and of the necessary IND-enabling toxicity studies.

### Example 12

### Intravenous Dose Escalation Studies with Combinatorial Treatment with scAAV9.U7-ACCA and 6-methyl-prednisolone (PDN)

The purpose of this study was to determine the effect of combinatorial treatment of scAAV9.U7.ACCA and 6-methyl-prednisolone (PDN). Dup2 mice (2 months of age) were treated with either 4.7E14 vg/kg scAAV9.U7.ACCA alone or in combination with intraperitoneal injections of PDN (IP,12 mg/kg/day) five times per week for 3 weeks.

### Western Blot Analysis

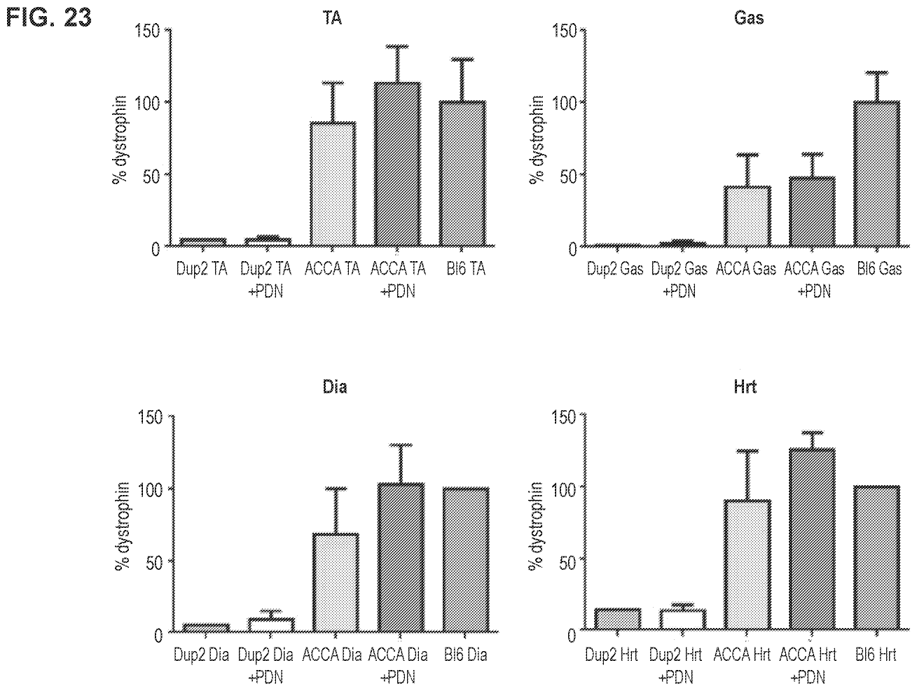

Protein was isolated from 5 muscles (TA, Gas, Triceps, Diaphragm and Heart) from each mouse and quantification was performed using Western blots following 12-14 weeks of treatment with scAAV9.U7.ACCA either alone or in combination with one month of treatment with PDN (FIG. 23). Dystrophin was present following treatment with scAAV9.U7.ACCA in every muscle and a one-fold increase was seen in some muscle with PDN addition. Following 12 weeks of scAAV9.U7.ACCA alone or in combination with one month of PDN treatment, 90% of WT levels was observed in the heart with scAAV9.U7.ACCA treatment alone and 125% with scAAV9.U7.ACCA+PDN. 68% of WT levels were observed in the diaphragm with scAAV9.U7.ACCA treatment alone and 103% with scAAV9.U7.ACCA+PDN.

### Immunofluorescence

Immunofluorescence staining of dystrophin (dys) was performed on all tissue samples to determine level of protein restoration following systemic delivery of 4.7×10¹⁴vg/kg of scAAV9.U7.ACCA alone or in combination with PDN. Staining showed proper sarcolemmal localization of the dystrophin protein at all doses when present (results not shown). All muscles show nearly complete restoration of scAAV9.U7.ACCA alone or in combination with PDN.

### Muscle Force

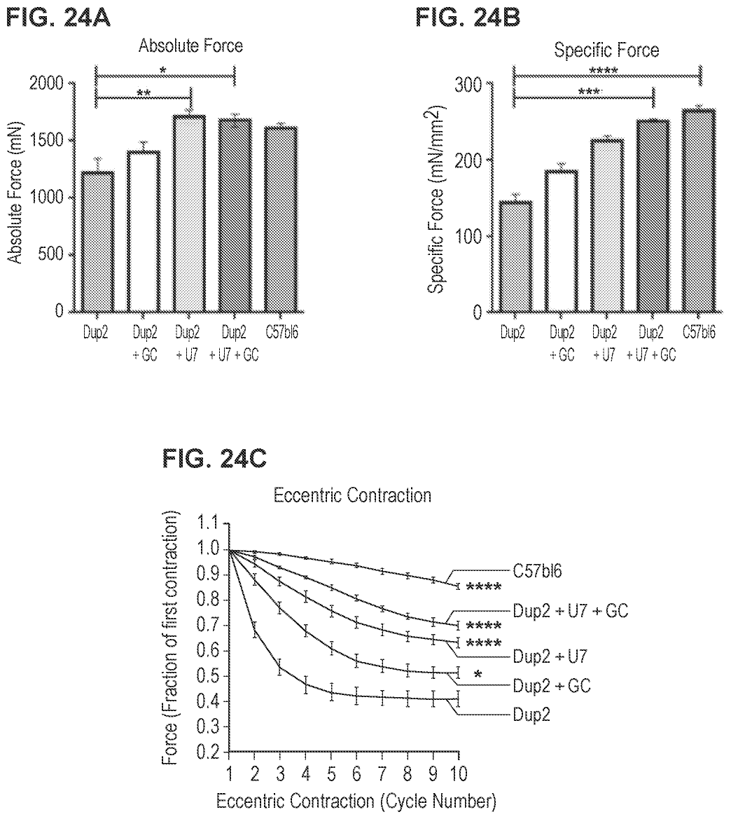

Tibialis Anterior (TA) muscle strength of mice was assessed through in vivo force analysis following systemic delivery of 4.7×10¹⁴vg/kg of scAAV9.U7.ACCA alone or in combination with PDN. Both absolute and specific force increased following treatment of scAAV9.U7.ACCA alone or in combination with PDN (FIGS. 24A-B). In addition, there was also improvement in force drop following repeated eccentric contractions (FIG. 24C).

Following establishment of the MED (see Example 11), a combination regime of scAAV9.U7.ACCA with PDN was evaluated. Animals were treated at 2 months of age with scAAV9.U7.ACCA followed by intraperitoneal injections of PDN at 4 months of age, 5 times per week. The data demonstrate that treatment with PDN leads to an increase in the level of dystrophin expression following scAAV9.U7.ACCA in the majority of muscles analyzed. Importantly, treatment with PDN alone does not induce dystrophin expression. Additionally, the increase in dystrophin levels led to improved force in the TA muscle.

To summarize, combination of scAAV9.U7.ACCA with PDN not only improves dystrophin expression in most muscle, but also partially improves muscle force. These results are important as most Duchenne muscular dystrophy patients are treated with PDN. This result indicates that the 5% of patients that are eligible for treatment with scAAV9.U7.ACCA would benefit from continuing their current PDN usage due to the synergistic effect of scAA9.U7.ACCA and PDN.

### Example 13

### Expression of Dystrophin after Intravenous Treatment of Human Patients using the scAAV9.U7-ACCA Vector

Exon duplications that cause Duchenne muscular dystrophy (DMD) are promising candidates for exon skipping therapies because skipping a single exon copy should result in wild-type transcript and full-length dystrophin expression. As described herein above, a therapeutic exon skipping viral vector (scAAV9.U7-ACCA) comprising four copies of a modified U7snRNA containing antisense sequences targeting the splice donor (2 copies) and splice acceptor (2 copies) of the DMD exon 2, the most commonly duplicated exon, was created. Following dose finding studies in Dup2 mice and after demonstrating lack of toxicity in non-human primates, a first-in-human clinical trial (NCT04240314) was initiated. As discussed herein above, if one of the duplicated exons is skipped, full length dystrophin should be expressed by this therapy. If both exons are skipped, a highly functional N-deleted isoform of dystrophin is expressed. This example provides the planned 3-month post-infusion data in the first two human subjects, i.e., two boys, treated with 3.0×10\hat{ }13 vg/kg scAAV9.U7-ACCA.

Both boys tolerated the treatment well, with only transient nausea and vomiting. No severe adverse events (SAEs) have been experienced to date and no biochemical measures of hepatic or other toxicity were seen. Muscle biopsy at 3 months shows expression of apparently full-length protein, quantified by Western blot at levels of >6% in the younger (9.0 yrs) subject and 1-2% in the older (13.7 yrs) subject, possibly reflecting differences in myofiber transfection due to differing degrees of dystrophic skeletal muscle changes. Consistent with dystrophin expression, serum CK decreased from 13,495 u/L to 560 u/L at 3 months post-injection in subject 1 and from 5,103 u/L to 947 u/L at 3 months in subject 2. Functional outcome measures remained stable or improved. These findings represent the first therapeutic expression of apparent full-length dystrophin in a human gene therapy trial and support continued clinical investigation.

### Subjects (Participants)

U7-Dup2-01 is a 9-year-old male with a confirmed duplication of exon 2 in the DMD gene. Symptoms were first noticed around age 3, falling more frequently, and genetic testing was done at age 5. He has been on daily deflazacort since Apr. 22, 2017 and his dose was increased to 18 mg total daily on Oct. 16, 2019.

U7-Dup2-02 is a 14-year-old male with a confirmed duplication of exon 2 in the DMD gene. He was born full term and early milestones were all on time. Symptoms were first noticed around age 4 or 4.5 with some toe walking, and generally appearing to be walking differently. He was diagnosed incidentally around age 4.5 and started deflazacort in January of 2014. He was on deflazacort 30 mg daily prior to study enrollment.

### Gene Transfer and Follow-Up

The subject, U7-Dup2-01, was started on prednisone at 1 mg/kg/day and a proton pump inhibitor (Prevacid) on Jan. 27, 2020 (Day −1). He was admitted to the PICU on the same day. Physical exam occurred January 28, and it was determined that it was safe to proceed with gene transfer. The drug was ordered and after a time-out, infusion was started at 11:03 am, and completed at 12:33 pm. The vector was given at a dose of 3.0×10¹³ vg/kg through a peripheral IV in the PICU room. There were no observed adverse reactions to the infusion. Vital signs were stable, and physical exams were unchanged each day and the subject was discharged from the hospital on Jan. 30, 2020 (Day 2). Day 7 follow-up examination occurred on February 4; Day 14 follow-up occurred on February 11; and Day 30 follow-up occurred on February 27. Labs were drawn for Day 45 on March 16, and the subject returned to Nationwide Children's Hospital (NCH) for Day 60 visit on March 27. Day 75 labs were drawn on April 14, Day 90 labs were drawn on April 21, and the 2-week Post Steroid return to baseline blood draw occurred on April 29. Due to COVID-19, the Day 90 visit consisted only of a telehealth physical exam and blood draw, and other missed assessments were scheduled to be completed at the Day 120 time point on May 26-28. The Day 120 visit occurred successfully, and after Day 150 labs were drawn on Jun. 29, 2020, the subject returned to NCH for the six-month visit on Jul. 28, 2020. The subject is due back for the one-year visit in late January 2021.

The subject, U7-Dup2-02, was admitted to the PICU on Apr. 6, 2020 (Day -1). The subject was started on prednisone at 1mg/kg/day and continued on his home omeprazole. Physical exam occurred on April 7, and it was determined that it was safe to proceed with gene transfer. The drug was ordered and infused on April 7 over 90 minutes. The vector was given at a dose of 3.0×10¹³ vg/kg through a peripheral IV in the PICU room. There were no visible changes at the infusion site and the infused was well tolerated. The subject was discharged from the hospital on April 9. Day 7 follow-up examination occurred on April 14; Day 14 follow-up occurred on April 21; and Day 30 follow-up occurred on May 5. In addition to blood draws performed on Day 45, 75, 120, and 150, the subject has returned to Nationwide Children's Hospital (NCH) for the Day 60 visit on June 9, 2020 and the Day 90 visit on Jul. 7, 2020, and he is scheduled to return to NCH for the six month visit on Oct. 8,2020.

### Post-Treatment Data

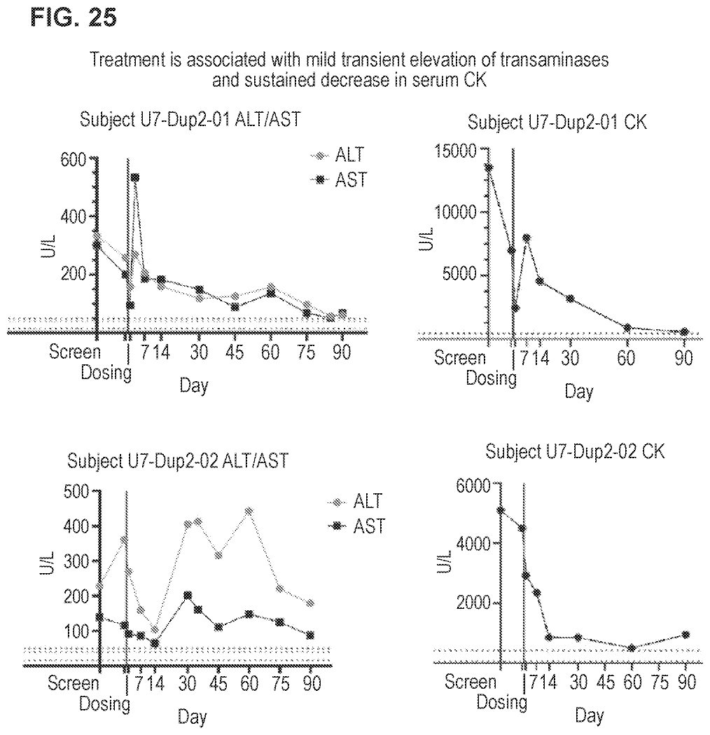

Treatment is associated with mild transient elevation of transaminases, alanine transaminase (ALT) and aspartate transaminase (AST) and sustained decrease in serum creatinine kinase (CK) (FIG. 25). There was a 95% decrease in serum CK level from baseline (13,495 to 560 u/L) in U7-Dup2-01. There was an 81% decrease in serum CK level from baseline (5,103 to 947 u/L).

TABLE 7
Functional outcomes.

| | Subject 1 | | | Subject 2 | | |
|---|---|---|---|---|---|---|
| | pre | post | % change | pre | post | % change |
| Functional | | | | | | |
| NSAA | 25 | 24 | | 22 | 22 | |
| FVC % p | 120 | 126 | 5% | 121 | 125 | 3% |
| 100 m % p | 54.3 | 62.8 | 16% | 58.3 | 27.7 | −2% |
| MVICT | | | | | | |
| R Knee Ext | 6.9 | 10.9 | 58% | 5.8 | 6.2 | 7% |
| R Knee Flex | 4.8 | 4.9 | 2% | 7 | 8.7 | 24% |
| L Knee Ext | 6.9 | 5.8 | −16% | 6.3 | 7.3 | 16% |
| L Knee Flex | 4.9 | 3.4 | −31% | 6 | 6.9 | 15% |

Functional outcomes in the two human subjects were largely stable or improved, as measured by the North Start Ambulatory Assessment (NSA) test, the forced vital capacity (FVC) test, the 100-meter timed test (100 m), and maximal voluntary isometric contraction testing (MVICT) (Table 7).

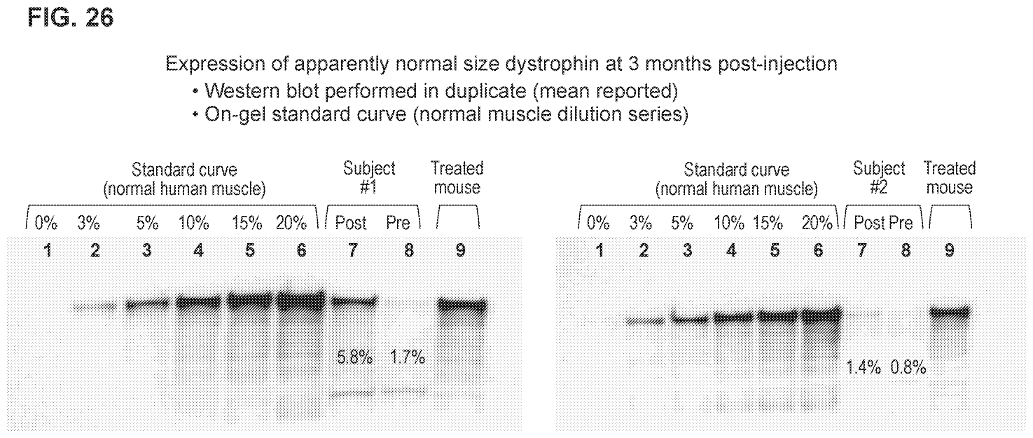

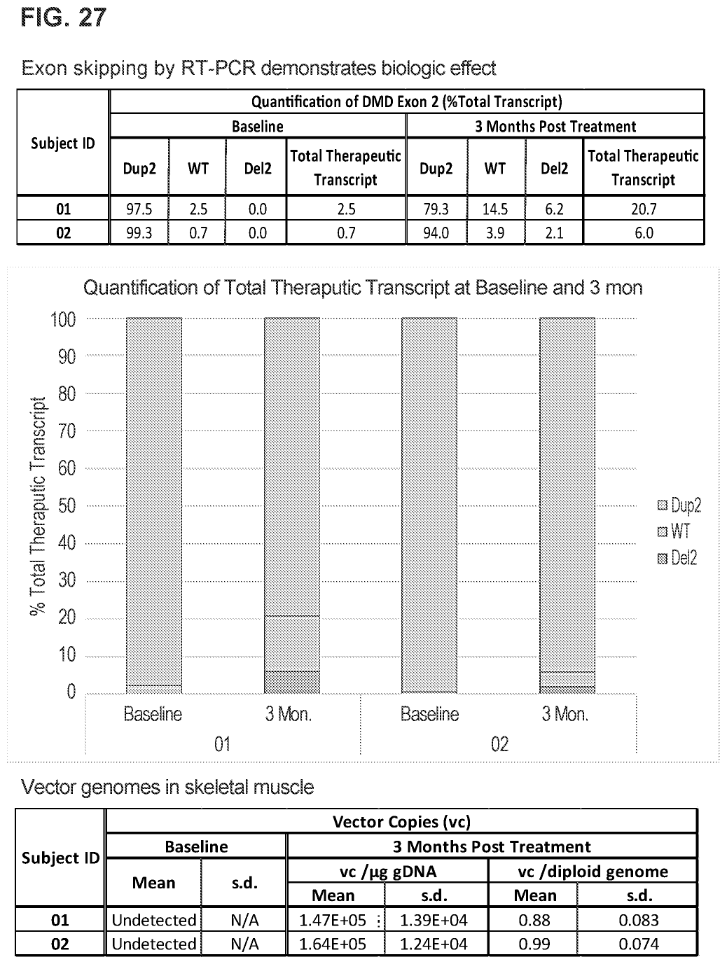

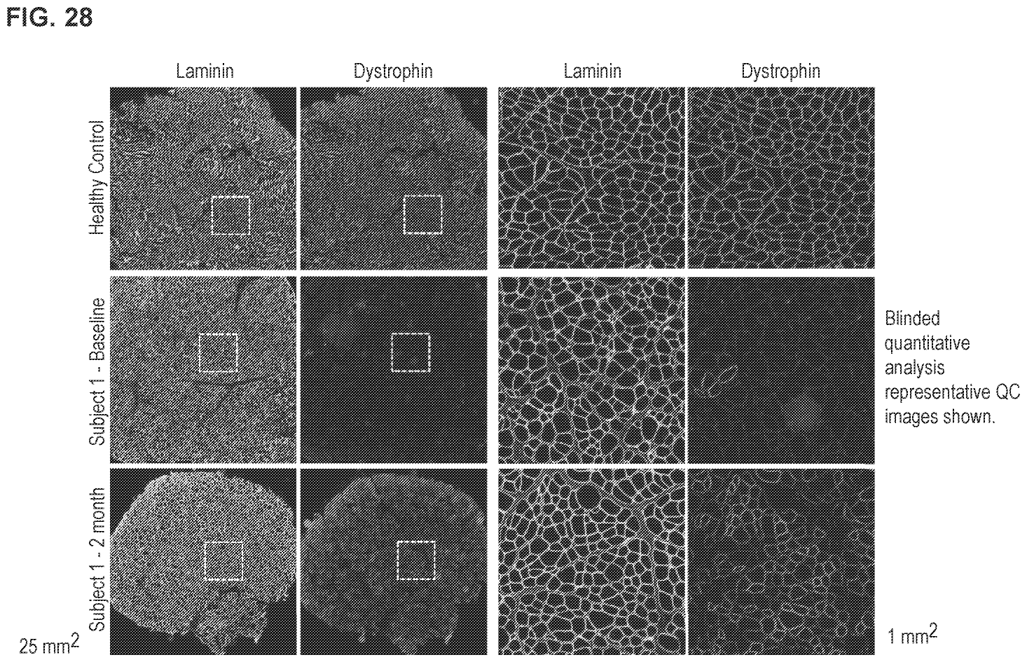

The expression of apparently normal size dystrophin at 3 months post-injection was observed in both subjects (FIG. 26). FIG. 26 shows on-gel standard curve (normal muscle dilution series). Western blot was performed in duplicate and the mean is reported. FIG. 27 shows exon skipping by RT-PCR demonstrated a biologic effect. At 3 months post-treatment, there was an increase in both WT and Del2 transcripts over baseline, and an increase in total therapeutic transcript in both subjects over baseline. Quantification of vector genomes in skeletal muscle 3 months post-treatment also is shown. FIG. 28 shows that dystrophin expression was markedly improved in subject 1 at 3 months post-treatment over baseline.

### Conclusion

Intravenous administration of scAAV9.U7.ACCA at a dose of 3×10¹³ vg/kg was well tolerated in both human subjects and there have been no severe adverse effects to date. At the 3 month data point, CK levels had markedly improved and functional outcomes were stable or slightly improved. An increase in dystrophin expression was clearly seen in the younger subject, representing the first example of apparent full-length dystrophin expression from gene therapy in DMD. This data supports further clinical investigation in additional subjects and dose escalation.

### Example 14

### Continued Follow Up of Human Patients After Intravenous Treatment using the SCAAV9.U7-ACCA Vector

Additional clinical data has become available from longer term follow up of the two subjects enrolled in the first-in-human study described in Example 13 above. This example provides the planned 18-month and 12-month post-infusion data in the first two human subjects, i.e., two boys treated with 3.0×10\hat{ }13 vg/kg scAAV9.U7-ACCA.

To date, both boys have tolerated the treatment well, with only transient nausea and vomiting. No severe adverse events (SAEs) have been experienced to date and no biochemical measures of hepatic or other toxicity were seen.

There was a 95% decrease in serum CK level from baseline to 3 months post-injection (13,495 to 560 u/L) in one subject, i.e., U7-Dup2-01. As of 18 months post-injection, the serum CK level in patient identified as U7-Dup2-01 has increased to 4237 U/L, which is a 69% decrease from screening or baseline. For U7-Dup2-02, the other subject, there was an 81% decrease in serum CK level from baseline to 3 months post-infusion (5,103 to 947 u/L). As of 12 months post-injection, the serum CK level in patient identified as U7-Dup2-02 has increased to 1458 U/L, which is a 71% decrease from baseline. However, the overall decrease in serum CK levels from baseline are sustained over time in both subjects.

Analysis of dystrophin expression has not yet been unblinded, but one of the subjects has shown dystrophin protein expression of 7% of normal at 12 months post-injection.

### Conclusion

To date, the intravenous administration of scAAV9.U7.ACCA at a dose of 3.0×10\hat{ }13 vg/kg was well tolerated in both human subjects and there have been no severe adverse effects to date. An increase in dystrophin expression was clearly seen in the one subject, representing the first example of apparent full-length dystrophin expression from gene therapy in DMD. The decreases in serum CK levels from baseline are sustained over time in both subjects. This data supports further clinical investigation in these two subjects as well as in additional subjects, and future dose escalation studies.

While the disclosure has been described in terms of specific embodiments, it is understood that variations and modifications will occur to those skilled in the art. Accordingly, only such limitations as appear in the claims should be placed on the disclosure.

All documents referred to in this application are hereby incorporated by reference in their entirety.

1-7. (canceled)

8. A method of treating, preventing or ameliorating a muscular dystrophy in a subject in need thereof comprising the step of administering to the subject a recombinant adeno-virus associated (rAAV) comprising a nucleic acid comprising a nucleotide sequence selected from the group consisting of:
(a) a nucleotide sequence comprising at least 80% identity to the sequence set forth in SEQ ID NO: 1;
(b) a nucleotide sequence complementary to the nucleotide sequence comprising at least 80% identity to the sequence set forth SEQ ID NO: 1;
(c) a nucleotide sequence comprising the sequence set forth in SEQ ID NO: 1; and
(d) a nucleotide sequence complementary to the nucleotide sequence comprising the sequence set forth in SEQ ID NO: 1, wherein the rAAV is administered at a dose of about 1.0×10¹⁰ vg/kg to about 1.0×10¹⁶ vg/kg.

9. The method of claim 8 wherein the dose is about 1.0×10¹¹ vg/kg to about 1.0×10¹⁵ vg/kg.

10. The method of claim 8 wherein the dose is about 1.0×10¹³ vg/kg to about 5.0×10¹³ vg/kg.

11. The method of claim 8 wherein the dose is about 2.0×10¹³ vg/kg to about 4.0×10¹³ vg/kg.

12. The method of claim 8 wherein the dose is about 3.0×10¹³ vg/kg.

13-19. (canceled)

20. The method of claim 8 wherein the rAAV is administered via a systemic route.

21. The method of claim 21 wherein the systemic route is by injection, infusion or implantation.

22. The method of claim 20 wherein the systemic route is an intravenous route.

23. The method of claim 21 wherein the rAAV is administered by infusion over approximately one hour.

24. The method of claim 8 wherein the muscular dystrophy is Duchenne Muscular Dystrophy or Becker Muscular Dystrophy.

25. The method of claim 8 wherein the level of functional dystrophin gene expression or protein expression in a cell of the subject is increased after administration of the rAAV as compared to the level of functional dystrophin gene expression or protein expression before administration of the rAAV.

26. (canceled)

27. The method of claim 8 wherein the level of serum creatinine kinase is decreased after administration of the rAAV as compared to the level of serum creatinine kinase before administration of the rAAV.

28. (canceled)

29. The method of claim 8 wherein muscular dystrophy progression in the subject is delayed or wherein muscle function in the subject is improved after administration of the rAAV as measured by the six minute walk test, time to rise test, ascend 4 steps test, ascend and descend 4 steps test, North Star Ambulatory Assessment (NSAA), the forced vital capacity (FVC) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

30. The method of claim 8 further comprising administering a second or combination therapy.

31. The method of claim 30 further comprising administering a glucocorticoid.

32-81. (canceled)

82. The method of claim 8 wherein the rAAV is rAAV1, rAAV2, rAAV3, rAAV4, rAAV5, rAAV6, rAAV7, rAAV8, rAAV9, rAAV10, rAAV11, rAAV12, rAAV13, rAAV-anc80, rAAV rh.74, rAAV rh.8, rAAVrh. 10, or rAAV-B1.

83. The method of claim 8 wherein the rAAV is rAAV9.

84. The method of claim 8 wherein the rAAV is self-complementary.
